(19) United States
(12) Patent Application Publication
(10) Pub. No.: US 2026/0176704 A1
(43) Pub. Date: Jun. 25, 2026

(54) BIOMARKERS FOR CANCER DETECTION

(71) Applicant: Nexosome Oncology LLC, Louisville, KY (US)

(72) Inventors: Todd HEMBROUGH, Sevierville, TN (US); Alan M. EZRIN, Sarasota, FL (US); Zachary OPHEIM, Raleigh, NC (US); Cheryl BANDOSKI, Raleigh, NC (US); Poorva MUDGAL, Mebane, NC (US)

(21) Appl. No.: 19/390,351

(22) Filed: Nov. 14, 2025

Related U.S. Application Data
(63) Continuation of application No. PCT/US2024/030114, filed on May 17, 2024.
(60) Provisional application No. 63/467,305, filed on May 17, 2023.
(60) Provisional application No. 63/575,608, filed on Apr. 5, 2024.

(51) Int. Cl. C12Q 1/6886

(52) U.S. Cl. CPC C12Q 1/6886; C12Q 2600/118

(57) ABSTRACT

The present disclosure relates to biomarker sets for cancer detection, as well as machine learning and algorithmic methods for identifying the biomarkers sets, and machine learning and algorithmic methods for using the biomarkers sets for cancer detection.

### CROSS REFERENCE TO RELATED APPLICATIONS

This application is a continuation of International Patent Application No. PCT/US2024/030114, filed May 17, 2024, which claims priority to U.S. Provisional Patent Application No. 63/467,305 filed on May 17, 2023, and U.S. Provisional Patent Application No. 63/575,608 filed on Apr. 5, 2024, the contents of each of which are hereby incorporated in their entirety by this reference.

### BACKGROUND

Microparticles are small, typically nano-scale (sub-micron), vesicular bodies released from cells and containing various biomolecules such as proteins, lipids, and nucleic acids. Microparticles are found generally in all biological fluids including blood, urine, and saliva. Microparticles may be of different cellular origins, and may include, by way of example, extracellular vesicles secreted by cells (e.g., released into the extracellular space through fusion of multivesicular bodies with the plasma membrane), exosomes, lipid rafts, or portions of cell membrane from degraded, damaged, or dying cells. Microparticles can be isolated or enriched from a biological sample through various methods, such as but not limited to size-exclusion chromatography or centrifugation.

Microparticles were first discovered in the 1980s and were initially thought to be cellular debris. However, they are now understood to be involved in intercellular communication and play a role in various physiological and pathological processes. Microparticles can transfer biomolecules such as proteins and nucleic acids between cells, thereby influencing the recipient cell's behavior. In the case of cancer, it has been shown that, cancerous cells can release microparticles that contain oncogenic proteins and RNA, which can be taken up by neighboring cells and contribute to the development and progression of cancer. It has also been shown that microparticles released from cancerous cells and associated myeloid cells in a tumor microenvironment can be derived from multiple biological fluids.

It has been the hope that microparticle-derived biomarkers can provide diagnostic, prognostic and stratification markers of cancer and drug responsiveness thereof. However, in practice, the usefulness of such biomarkers has been limited by an inability to isolate microparticles and detect microparticle-derived biomarkers with sufficient yield and reproducibility. A number of approaches have been utilized to recover and assess the presence of biomarkers in isolated microparticles. However, to date, such efforts have been limited by relatively large background signals and an inability to evaluate signal beyond the most abundant proteins.

Thus, there is a need for refined biomarker sets for the diagnosis, prognosis, and stratification of cancer states and, computational methods related to the same. Provided herein are machine learning and algorithmic methods and biomarker sets that address this need.

Patents, patent applications, patent application publications, journal articles and protocols referenced herein are incorporated by reference.

### SUMMARY

The present disclosure relates to biomarker sets for cancer detection, as well as machine learning and algorithmic methods for identifying the biomarkers sets, and machine learning and algorithmic methods for using the biomarkers sets for cancer detection.

In one aspect, provided herein is a method for analyzing a biological fluid sample of a subject, the method comprising:
(a) providing a microparticle preparation prepared from a biological fluid sample from a subject, wherein the biological fluid sample comprises microparticles;
(b) assaying the expression level of two or more proteins from the microparticle preparation, to yield a data set comprising respective quantitative measures of each of the two or more proteins;
(c) inputting the data set to a trained classifier that is configured to generate a classification of said sample as positive or negative for the cancer at an accuracy of at least 80%; and
(d) electronically outputting a report that identifies said classification of the sample as positive or negative for the cancer.

In some embodiments, the trained classifier is configured to generate the classification of said sample as positive or negative for the cancer at an accuracy of at least 90%, at least 95%, at least 96%, at least 97%, at least 98%, or at least 99%.

In some embodiments, the trained classifier was trained with training data obtained from a plurality of training samples, and wherein the training samples are microparticle preparations obtained from biological fluid samples from known cancer patients and known non-cancer subjects. Optionally, the training data set comprises, for each of the plurality of training samples: (a) a training classification of cancer or non-cancer; and (b) a quantitative measure of at least the two or more proteins. Optionally, the trained classifier is an algorithm comprising a plurality of coefficients, each of the plurality of the coefficients being associated with one of the two or more proteins, and wherein the algorithm is configured to generate the classification based on the data set comprising the respective quantitative measures of the two or more proteins and the plurality of coefficients.

In some embodiments, the two or more proteins are selected from any one of Tables 2.1, 3.1, 4.1, 5.1, 6.1, 7.1-7.3, 8.2, 9.2, 9.5, 9.8, and 9.11.

In some embodiments, the two or more proteins are selected from Tables 2.1 or 8.2. Optionally, the two or more proteins comprise a multiplex of proteins selected from a plurality of multiplexes provided in Table 2.2 or Table 8.3. Optionally, the multiplex of proteins comprises at least one protein selected from Table 8.4. Optionally, the multiplex of proteins comprises one or both of CO3 and PROS. Optionally, the cancer is selected from the group consisting of: ovarian cancer, colorectal cancer, lung cancer, and breast cancer.

In some embodiments, the two or more proteins are selected from Table 9.14. Optionally, the two or more proteins comprise a multiplex of proteins selected from a plurality of multiplexes provided in Table 9.15. Optionally, the multiplex of proteins comprises at least one protein selected from Table 9.16. Optionally, the multiplex of proteins comprises one, two, or three proteins out of HEP2, C4BPB, B3AT, and PHLD. Optionally, the cancer is selected from the group consisting of: ovarian cancer, colorectal cancer, lung cancer, and breast cancer.

In some embodiments, the cancer is breast cancer, and the two or more proteins are selected from Table 3.1 or Table 9.11. Optionally, the two or more proteins comprise a multiplex of proteins selected from a plurality of multiplexes provided in Table 9.12. Optionally, the multiplex of proteins comprises at least one protein selected from Table 9.13. Optionally, the multiplex of proteins comprises one, two, or three proteins out of PHLD, FIBA, FIBG, and HEP2.

In some embodiments, the cancer is lung cancer, and the two or more proteins are selected from Table 5.1 or Table 9.5. Optionally, the two or more proteins comprise a multiplex of proteins selected from a plurality of multiplexes provided in Table 9.6. Optionally, the multiplex of proteins comprises at least one protein selected from Table 9.7. Optionally, the multiplex of proteins comprises one, two, or three proteins out of HEP2, C4PBP, and PROS.

In some embodiments, the cancer is colorectal cancer, and the two or more proteins are selected from Table 4.1 or Table 9.8. Optionally, the two or more proteins comprise a multiplex of proteins selected from a plurality of multiplexes provided in Table 9.9. Optionally, the multiplex of proteins comprises at least one protein selected from Table 9.10. Optionally, the multiplex of proteins comprises one, two, or three proteins out of ClQB, APOA4, PROS, and ECM1.

In some embodiments, the cancer is ovarian cancer, and the two or more proteins are selected from any one of Tables 6.1, 7.1, 7.2, 7.3, and 9.2. Optionally, the two or more proteins comprise a multiplex of proteins selected from a plurality of multiplexes provided in Table 9.3 Optionally, the multiplex of proteins comprises at least one protein selected from Table 9.4. Optionally, the multiplex of proteins comprises one, two, or three proteins out of C4BPB, APOA4, PCGBP, PHLD, HABP2, and FIBA.

In some embodiments, the two or more proteins comprise a lipid metabolism protein, an extracellular matrix protein, or an innate immunity protein. Optionally, the two or more proteins comprise the lipid metabolism protein, and the lipid metabolism protein is PON1. Optionally, the two or more proteins comprise the hemostasis protein, and the hemostasis protein is Factor XI or Platelet Factor 4. Optionally, the two or more proteins comprise the extracellular matrix protein, and the extracellular matrix protein is Tenascin-C or Thrompospondin-1. Optionally, the two or more proteins comprise the innate immunity protein, and the innate immunity protein is, or is a subunit of: Complement Factor H, Complement Component 1 Subcomponent S, or Complement Component 1q.

In another aspect, provided herein is a method for determining presence of a cancer in a subject, the method comprising:
(a) providing a microparticle preparation prepared from a biological fluid sample from a subject, wherein the biological fluid sample comprises microparticles;
   (b) quantifying two or more proteins in the fraction; and
   (c) based on the quantification of the two or more proteins, determining the presence of the cancer in the subject,
wherein the two or more proteins are selected from any one of Tables 2.1, 3.1, 4.1, 5.1, 6.1, 7.1-7.3, 8.2, 9.2, 9.5, 9.8, 9.11.

In some embodiments, the two or more proteins are selected from Tables 2.1 or 8.2. Optionally, the two or more proteins comprise a multiplex of proteins selected from a plurality of multiplexes provided in Table 2.2 or Table 8.3. Optionally, the multiplex of proteins comprises at least one protein selected from Table 8.4. Optionally, the multiplex of proteins comprises one or both of CO3 and PROS. Optionally, the cancer is selected from the group consisting of: ovarian cancer, colorectal cancer, lung cancer, and breast cancer.

In some embodiments, the two or more proteins are selected from Table 9.14. Optionally, the two or more proteins comprise a multiplex of proteins selected from a plurality of multiplexes provided in Table 9.15. Optionally, the multiplex of proteins comprises at least one protein selected from Table 9.16. Optionally, the multiplex of proteins comprises one, two, or three proteins out of HEP2, C4BPB, B3AT, and PHLD. Optionally, the cancer is selected from the group consisting of: ovarian cancer, colorectal cancer, lung cancer, and breast cancer.

In some embodiments, the cancer is breast cancer, and the two or more proteins are selected from Table 3.1 or Table 9.11. Optionally, the two or more proteins comprise a multiplex of proteins selected from a plurality of multiplexes provided in Table 9.12. Optionally, the multiplex of proteins comprises at least one protein selected from Table 9.13. Optionally, the multiplex of proteins comprises one, two, or three proteins out of PHLD, FIBA, FIBG, and HEP2.

In some embodiments, the cancer is lung cancer, and the two or more proteins are selected from Table 5.1 or Table 9.5. Optionally, the two or more proteins comprise a multiplex of proteins selected from a plurality of multiplexes provided in Table 9.6. Optionally, the multiplex of proteins comprises at least one protein selected from Table 9.7. Optionally, the multiplex of proteins comprises one, two, or three proteins out of HEP2, C4PBP, and PROS.

In some embodiments, the cancer is colorectal cancer, and the two or more proteins are selected from Table 4.1 or Table 9.8. Optionally, the two or more proteins comprise a multiplex of proteins selected from a plurality of multiplexes provided in Table 9.9. Optionally, the multiplex of proteins comprises at least one protein selected from Table 9.10. Optionally, the multiplex of proteins comprises one, two, or three proteins out of ClQB, APOA4, PROS, and ECM1.

In some embodiments, the cancer is ovarian cancer, and the two or more proteins are selected from any one of Tables 6.1, 7.1, 7.2, 7.3, and 9.2. Optionally, the two or more proteins comprise a multiplex of proteins selected from a plurality of multiplexes provided in Table 9.3 Optionally, the multiplex of proteins comprises at least one protein selected from Table 9.4. Optionally, the multiplex of proteins comprises one, two, or three proteins out of C4BPB, APOA4, PCGBP, PHLD, HABP2, and FIBA.

In some embodiments, the two or more proteins comprise a lipid metabolism protein, an extracellular matrix protein, or an innate immunity protein. Optionally, the two or more proteins comprise the lipid metabolism protein, and the lipid metabolism protein is PON1. Optionally, the two or more proteins comprise the hemostasis protein, and the hemostasis protein is Factor XI or Platelet Factor 4. Optionally, the two or more proteins comprise the extracellular matrix protein, and the extracellular matrix protein is Tenascin-C or Thrompospondin-1. Optionally, the two or more proteins comprise the innate immunity protein, and the innate immunity protein is, or is a subunit of: Complement Factor H, Complement Component 1 Subcomponent S, or Complement Component 1q.

In another aspect, provided herein is a method for determining presence of a cancer-induced host immunomodulated environment in a subject, the method comprising:
(a) providing a microparticle preparation from a biological fluid sample from the subject;
(b) quantifying two or more proteins in the microparticle preparation, wherein the two or more proteins include at least one antigen presenting cell (APC) marker or at least one tumor immune suppressor; and
(c) based on the quantification of the two or more proteins, determining the presence of the cancer-induced immunomodulation in the subject.

Optionally, the at least one APC marker comprises colony stimulating factor 1 receptor. Optionally, the at least one tumor immune suppressor comprises Fibrinogen-like protein 1.

In another aspect, provided herein is a method of treating cancer in a subject, the method the method comprising:
(a) providing a microparticle preparation from a biological sample from the subject;
(b) quantifying two or more proteins in the microparticle preparation, wherein the two or more proteins include at least one antigen presenting cell (APC) marker or at least one tumor immune suppressor;
(c) determining presence of cancer-induced immunomodulation in the subject based on the quantification of the two or more proteins; and
(d). administering an effective amount of an immune response modulator to the subject based on the determination of the presence of cancer-induced immunomodulation in the subject, thereby treating the cancer.

Optionally, the at least one APC marker comprises colony stimulating factor 1 receptor. Optionally, the at least one tumor immune suppressor comprises Fibrinogen-like protein 1.

In another aspect, provided herein is a method comprising:
a) providing a plurality of microparticle preparations, each of the plurality of microparticle preparations being prepared from a plasma or serum sample from one of a plurality of subjects, the plurality of subjects comprising cancer patients and non-cancer subjects;
b) using mass spectrometry, determining quantitative measures of a plurality of proteins in each of the plurality of microparticle preparations, wherein the plurality of proteins are selected from: the proteins of any one of Tables 2.1, 2.2, 0.1, 4.1, 5.1, 6.1, 7.1, 7.2, 7.3, 8.2-8.4, and 9.2-9.16.
c) preparing a training data set indicating, for each sample, values indicating:
   (i) classification of cancer class or non-cancer class; and
   (ii) quantitative measures, respectively, of the plurality of proteins; and
d) training a classifier on the training data set, wherein training generates one or more classification rules that classify a new sample as belonging to the cancer class or the non-cancer class.

In another aspect, provided herein is a computer system comprising:
(a) a processor; and
(b) a memory, coupled to the processor, the memory storing a module comprising:
   (i) test data for a sample from a subject, the test data including values indicating a quantitative measure of two or more proteins in a microparticle preparation from a biological fluid sample, wherein the two or more proteins are selected from the proteins of any one of Tables 2.1, 2.2, 3.1, 4.1, 5.1, 6.1, 7.1, 7.2, 7.3, 8.2-8.4, and 9.2-9.16;
   (ii) a trained classifier configured to, based on the test data, classify the subject as having a cancer or not having the cancer; and
   (iii) computer executable instructions for implementing the classifier on the test data.

In some embodiments, the classifier is configured to have an accuracy of at least 80%, at least 90%, at least 95%, at least 96%, at least 97%, at least 98%, or at least 99%.

### BRIEF DESCRIPTION OF THE DRAWINGS

FIG. 1 shows a volcano plot that visually represents the distribution of all proteins identified in the study, in which the X-axis represents fold change of a given protein represented as a plot point, and the Y-axis represents the p-value of the given protein.

FIG. 2A shows a heat map of differentially expressed microparticle-associated proteins in microparticle preparations aggregating all 4 cancer types (ovarian, colorectal, breast and lung) evaluated vs. non-cancer subject microparticle preparations.

FIG. 2B represents the same data set as FIG. 2A, broken out into each of the cancer types included in the study.

FIG. 3 shows the results of an ELISA analysis of the protein C1q in microparticle preparations from different cancer cohorts vs. a non-cancer (“Control”) cohort.

FIG. 4A shows the ROC curve of Heparin Cofactor 2 (“HC2”), which had an AUC value of 0.926.

FIG. 4B shows the expression distribution of HC2, comparing expression in microparticle preparations from cancer patients (light gray; right) vs microparticle preparations from non-cancer (“Control”) subjects (dark gray; left).

FIG. 5 shows the collective ROC curve of the five biomarkers listed in Table 2.2.

FIG. 6 shows a Volcano Plot of differentially expressed proteins in microparticle preparations from ovarian cancer patients compared with non-cancer (“Control”) subjects.

FIGS. 7A-7Y show the expression distribution of ovarian cancer biomarkers listed in Table 7.1 obtained from microparticle preparations, between ovarian cancer (Ovarian) and non-cancer (“Control”) cohorts. FIG. 7A shows the expression distribution of biomarker SLC4A1. FIG. 7B shows the expression distribution of biomarker ANKI. FIG. 7C shows the expression distribution of biomarker SPTB. FIG. 7D shows the expression distribution of biomarker SNED1. FIG. 7E shows the expression distribution of biomarker EPB41. FIG. 7F shows the expression distribution of biomarker EPB42. FIG. 7G shows the expression distribution of biomarker SPTA1. FIG. 7H shows the expression distribution of biomarker B3GNT2. FIG. 7I shows the expression distribution of biomarker SRGN. FIG. 7J shows the expression distribution of biomarker APP. FIG. 7K shows the expression distribution of biomarker RNASE4. FIG. 7L shows the expression distribution of biomarker ADAMDEC1. FIG. 7M shows the expression distribution of biomarker BMP1. FIG. 7N shows the expression distribution of biomarker FTL. FIG. 7O shows the expression distribution of biomarker FAM234A. FIG. 7P shows the expression distribution of biomarker F10. FIG. 7Q shows the expression distribution of biomarker ENG. FIG. 7R shows the expression distribution of biomarker PF4. FIG. 7S shows the expression distribution of biomarker F11. FIG. 7T shows the expression distribution of biomarker MYH9. FIG. 7U shows the expression distribution of biomarker MGAT5. FIG. 7V shows the expression distribution of biomarker MADCAM1. FIG. 7W shows the expression distribution of biomarker FBLN7. FIG. 7X shows the expression distribution of biomarker PF4V1. FIG. 7Y shows the expression distribution of biomarker TGFBI.

FIG. 8 shows a Partial Least Squares discriminant (PLS-DA) analysis of the differential expression of biomarkers in microparticle preparations from ovarian cancer samples (top left cluster) vs non-cancer samples (bottom right cluster).

FIGS. 9A-9C shows the cancer-based differential expression, between cancer and non-cancer (“Control”) cohorts, of serum paraoxonase/arylesterase 1 (gene name PON1) in microparticle preparations, as measured through LC/MS quantification (FIG. 9A) or with ELISA (FIGS. 9B-9C).

FIGS. 10A-10C shows the cancer-based differential expression, between cancer and non-cancer (“Control”) cohorts, of complement factor H (gene name CFH) in microparticle preparations, as measured through LC/MS quantification (FIG. 10A) compared with ELISA (FIGS. 10B-10C).

FIG. 10D shows the lack of cancer-based differential expression of complement factor H (gene name CFH) as measured through LC/MS quantification compared with ELISA using native serum (not enriched for microparticles), (compared to the cancer-based differential expression when using microparticle preparations, as shown in FIG. 10C).

FIGS. 11A-11H, show the results of an ELISA analysis of a subset of the ovarian cancer biomarkers listed in Table 7.1, in microparticle preparations from cancer patients vs. non-cancer (“Control”) subjects.

FIG. 12A shows a visualization of the ranking of the RF-identified ovarian cancer biomarkers.

FIG. 12B shows a visualization of the RFE cross validation of ovarian cancer biomarkers.

FIG. 12C shows the expression distribution of SNED1, B3AT, ADEC1, SPTA1, and NOE2 proteins in microparticle preparations, as quantified by MS, between ovarian cancer (“Cancer”) and non-cancer (“Control”) cohorts.

FIG. 13A shows a ROC curve based on the ELISA quantification of Platelet Factor 4 in microparticle preparations.

FIG. 13B shows an expression distribution of Platelet Factor 4 in microparticle preparations based on ELISA quantification, between ovarian cancer and non-cancer (“normal”) cohorts.

FIG. 13C shows a confusion matrix of results shown in FIG. 13B.

FIGS. 14A-14B shows the results of an ELISA analysis of CSF1-R and FGL1, respectively, in microparticle preparations from non-cancer subjects, compared against microparticle preparations from a variety of cancer cohorts.

FIG. 15 shows examples of CD9 and CD63 Western Blots of in microparticle-enriched fractions of plasma.

### DETAILED DESCRIPTION

There is provided herein improved methods for detection, determination, diagnosis, or prognostication of one or more aspects of a cancer in a subject, based on multiplexed proteomics of microparticle-associated biomarkers. “Multiplexed proteomics” as used herein refers to the analysis of a quantitative measure of two or more biomarkers. The biomarkers may be proteins or fragments thereof. The aspects of cancer may include one or a combination of cancer type, cancer stage, cancer presence or recurrence, stratification of patient populations for assigning to a therapeutic regime or a therapeutic trial, longitudinal monitoring of cancer progression, and longitudinal monitoring of patient response to a therapeutic regime.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Applicant discloses herein methods of isolating microparticles from a subject and analyzing proteomic information from the isolated microparticles to determine one or more aspects of a cancer in the subject, such as the presence or recurrence of a cancer. Such analysis of the isolated microparticles may also be informative with regard to various clinical indications such as, for example, cancer diagnosis, classification, monitoring, and assessment of therapeutic efficacy.

In some embodiments, the analysis of proteomic information may involve a computational analysis and/or use of a computer system comprising a processor and a memory operably connected to the processor. The memory may store a module comprising test data for a sample from a subject, or test data for a plurality of samples, respectively, from each of a plurality of subjects. The module may further comprise a trained algorithm (e.g., a classifier) configured to classify the subject or plurality of subjects as having a cancer or not having the cancer based on the test data. The module may further comprise computer executable instructions for implementing the classifier on the test data. In some embodiments, the computer system may be implemented as a distributed cloud network, comprises a plurality of interconnected nodes, each node comprising a processor and a memory operably connected to the processor, that are configured to collaboratively execute computational tasks. In some embodiments, the computer system may be embodied as a standalone laptop or desktop computer, each comprising a processor and a memory operably connected the processor, as well as input/output interfaces for user interaction and peripheral connectivity.

### Definitions

In order to facilitate an understanding of the disclosure, selected terms used in the application will be discussed below.

“Diagnosis” as used herein may refer to the identification of a disease or likelihood of a disease in a test subject. In particular, “diagnosing cancer” as used herein may refer to the identification of cancer in a test subject not previously known to have a cancer, the identification of a cancer in a test subject known to have had the cancer previously (i.e., recurrence), or to the determination of whether a test subject has an increased likelihood or probability of having cancer. “Diagnosing cancer” may also refer to the identification or prediction of increased likelihood of a specific type of cancer in a test subject. “Diagnosing cancer” may also refer to the identification or prediction of cancer stage, cancer grade, age, physical symptoms, and medical history. The diagnosis of cancer may be based on information from two or more biomarkers, such as the expression levels of the protein biomarkers disclosed herein.

“Monitoring” as used herein may refer to the act of observing. Monitoring may include, for example, observing the expression level of a protein in a microparticle, optionally in a plurality of instances over a period of time. Monitoring may also refer to the observation of a physical characteristic such as, for example, the number of microparticles in a sample, optionally in a plurality of instances over a period of time.

“Microparticle” as used herein may refer to a small nano-scale (sub-micron) vesicular body released from cells and containing various biomolecules such as proteins, lipids, and nucleic acids. Microparticles may include, for example, endosome-derived exosomes, plasma membrane-derived shedding vesicles, microvesicles, extracellular particles, extracellular vesicles (EVs), exosomes, exomeres, small EVs, large EVs, apoptotic bodies, prostasomes, P2 and P4 particles, and outer membrane vesicles (OMVs).

A “microparticle-associated proteins” (MAPs) as used herein may include proteins associated with microparticles in one of a variety of ways. MAPs may refer to any protein that has been contained within a microparticle (also referred to as intra-vesicular protein), located on the surface of a microparticle, or trapped between aggregated microparticles (also referred to as an inter-vesicular protein). Some MAPs may be “intrinsic MAPs” that were originally found and/or expressed in the cells (“source cells”) from which the microparticle was released. Intrinsic MAPs may include membrane-bound proteins bound to a membrane of the microparticle, which is typically a portion of a membrane from the source cell. If the microparticles are vesicles with a lumen, the intrinsic MAPs may include intra-vesicular proteins comprised in the lumen of the vesicle, which may be, for example, a sampling of the intracellular environment of the source cell. In addition, MAPs may be “corona proteins” defining a microparticle's “microenvironment”, which are associated with the microparticle through external macromolecular interactions, e.g., protein-protein or receptor-ligand interactions. As such, corona proteins may be proteins that are not from the source cells of the microparticles, but rather “host proteins” found in the local environments in which the microparticles may have resided, or have traversed, within the subject (i.e. host) after being released from the source cell. By way of example, and without being limited by theory, if the microparticles are purified from the subject's plasma in a way (for example using methods provided herein) that preserves or retains the corona proteins, the MAPs may include a sampling of proteins found in the host's bloodstream, thus reflecting not simply the state of the source cells of the microparticles, but also reflecting an overall disease state of the host. In such a case, the host protein may be considered a host disease response protein. As such, for example, if the subject is suffering from a disease, e.g., cancer, the corona proteins may include proteins that reflect the subject's response to the cancer even if none or only a subset of the microparticles were released from cancer cells. A MAP includes both a protein while it is associated with a microparticle, as well as after the protein has been dissociated from the microparticle.

### Methods—General

In one aspect, the disclosure herein provides for methods of identifying cancer biomarkers based on the expression level of one or more MAPs a biological sample from cancer patients and non-cancer subjects. In another aspect, the disclosure herein also provides for methods of determining an aspect of a cancer in a subject (e.g., diagnosing or prognosticating the presence of a cancer), using cancer biomarkers quantified from a microparticle-enriched fraction, which cancer biomarkers may have been identified using the biochemical and computational methods described herein. As such, the methods of both aspects of the disclosure may include any one of: processes for preparing a microparticle-enriched fraction from a biological sample; isolating MAPs or fragments thereof from the microparticle-enriched fraction; quantifying one or more of the MAPs; and obtaining or receiving quantification data of the one or more MAPs or fragments thereof in the biological sample.

The methods of the disclosure may comprise providing a microparticle-enriched fraction from a biological sample from the subject, quantifying two or more proteins in the fraction, and determining an aspect of the cancer in the subject based on the quantification of the two or more proteins. The two or more proteins used for determining an aspect of a cancer in a subject may be referred to herein as a “cancer biomarker”.

### Samples Containing a Bodily Fluid

The methods of the disclosure may comprise extracting, obtaining, or providing a sample containing a bodily fluid of a subject. In some embodiments, the bodily fluid may be extracted from the subject directly. In some embodiments, the bodily fluid may have been extracted from a subject by a third party, which is then stored, for example in frozen storage, and the bodily fluid may be obtained from storage, or received from the third party. The bodily fluid sample may then be used as a source of microparticles, as described herein below.

Various samples containing a bodily fluid from a subject will be apparent to one of skill in the art and may be used in the methods disclosed herein. A bodily fluid may refer to, for example, a sample of fluid isolated from anywhere in the body of the subject, for example a peripheral location, including but not limited to, for example, blood or a fraction thereof (e.g., plasma, serum), urine, sputum, spinal fluid, pleural fluid, interstitial fluid, bile, glandular fluid, exudate, nipple aspirates, lymph fluid, respiratory droplets, intestinal, and genitourinary tracts, tears, saliva, breast milk, lacrimal fluid, fluid from the lymphatic system, semen, cerebrospinal fluid, intra-organ system fluid, ascitic fluid, tumor cyst fluid, synovial fluid, amniotic fluid, ocular fluid, ascites, bronchoalveolar lavage, and combinations thereof. The method of extraction or storage depends on the bodily fluid, and many such methods are known in the art. In some embodiments, the bodily fluid may be a dried bodily fluid that is reconstituted. In some embodiments, the bodily fluid may undergo various processing step prior to isolation or enrichment of microparticles. By way of example, the bodily fluid may be processed to remove cells, or macroscale solids through, e.g., filtration or centrifugation. In exemplary embodiments, the sample is blood, plasma or urine. If the sample is blood, the sample may be centrifuged to remove cellular material and debris such that a plasma or serum fraction is generated, which is then further process to enrich for microparticles, for example as described herein below.

Enrichment of Microparticles from a Sample

The methods of the present disclosure may comprise enriching or isolating microparticles from a biological sample. For example, a population of microparticles may be isolated from the sample according to any methods known to one of skill in the art (see, for example, Cocucci et al, Traffic 8, 2007:742-757; Simpson et al, Proteomics 8, 2008: 4083-4099; Diaz et al., J. Vis. Exp. (134), e57467, doi:10.3791/57467 (2018). In some embodiments, isolating microparticles may comprise isolating or enriching a given sub-population of microparticles, such as microparticles within a given range of diameters or molecular weights, or microparticles having a specific marker indicating, e.g., a specific class or source of the microparticles.

In certain embodiments, an exemplary method of isolating or enriching for microparticles involves size exclusion chromatography, although other methods of enrichment may be used alternative or in combination, including but not limited to serial centrifugation or ultracentrifugation (Raposo et al., J Exp Med 183, 1996: 1161-72), density gradients (e.g. sucrose density gradients), alternating current (AC) electrokinetic separation; electrophoresis (e.g. organelle electrophoresis), electroporation, anion exchange and/or gel permeation chromatography, magnetic activated sorting (e.g., using magnetic beads), filtration (e.g., microfiltration, nanomembrane ultrafiltration concentration), and microchips with microfluidic technology. Microparticles may also be, in the alternative or in combination, isolated from a sample using affinity capture or affinity capture methods in solution or solid phase. For example, these affinity methods may be immunoaffinity methods (e.g., immunoprecipitation), but in other embodiments, such methods employ other reagents which bind specifically to proteins. Various methods for the isolation of microparticles can be found, for example, in U.S. Pat. Nos. 6,899,863, 6,812,023, Taylor and Gercel-Taylor, Gynecol Oncol 110, 2008: 13-21, Cheruvanky et al, Am J Physiol Renal Physiol 292, 2007: F1657-61, and Nagrath et al, Nature 450, 2007: 1235-9. A sample that has undergone microparticle enrichment or isolation may be referred to herein as a “microparticle preparation”.

In certain embodiments, microparticles, either in enriched form or as found natively in the sample, can be contacted with a tissue-specific reagent to isolate microparticles derived from a specific tissue. Exemplary tissues of interest from which microparticles can be derived, and isolated in a tissue-specific manner, may include, for example, brain, adrenal gland, endocrine gland, pituitary, hypothalamus, parathyroid, uterus, heart, blood vessel, stomach, trachea, pharynx, gums, hair, scalp, subcutaneous tissue, fallopian tube, reproductive tract, urethra, skin, bone, stem cell, umbilical cord, placenta, lymphocyte, monocyte, macrophage, formed blood cell, smooth muscle, skeletal muscle, connective tissue, spinal cord, kidney, bladder, anus, bone, breast, prostate, lung, cervix, colon, rectum, uterus, esophagus, skin, liver, pharynx, mouth, neck, ovary, pancreas, lung, eye, intestine, mouth, thyroid, GI tract, and endometrium.

In certain embodiments, microparticles may be isolated from the sample with an organelle-specific reagent to isolate the microparticles derived from organelles of cells in a specific tissue. Particular organelles of interest may include, for example, plasma membrane, peroxisome, smooth ER, rough ER, lysosome, mitochondria, and nucleus. In some embodiments, each step is performed using multiple microparticle-specific reagents.

In some embodiments, the entire population of microparticles from the sample may be contacted with a reagent that binds to microparticles derived from multiple tissue types rather than one that binds microparticles derived from a specific tissue.

Once a population of microparticles has been isolated or enriched from a sample, the microparticles in the microparticle preparation may be subjected to further selection steps to isolate a subpopulation of microparticles from the more general microparticle population isolated from the sample. For example, a population of microparticles isolated from a sample may comprise cancer cell-derived and non-cancer cell-derived microparticles. In some embodiments, the population of microparticles may be subjected to a further selection step to isolate cancer cell-derived microparticles. Conversely, the population of microparticles may be subjected to a further selection step to isolate non-cancer cell-derived microparticles.

### Exemplary Enrichment Process: Size Exclusion Chromatography

In certain embodiments, the microparticles may be enriched from a biological sample using size exclusion chromatography (SEC). In certain embodiments, use of a SEC column to isolate microparticles allows for suspension of the resin beads, such as a mobile bead column or other resin beads, during washing. It has been discovered that such a column configuration results in significantly lower levels of background contamination and hence more sensitive levels of quantitation and overall yield of the desired target.

In certain embodiments, the column used in such methods includes a lower end including an outflow opening; a lower porous support; a layer of resin on the lower porous support; the resin having specific size exclusion for a population of microparticles; an upper porous support; and an upper end including an inflow opening, wherein the resin between the lower porous support and the upper porous support is structured and arranged to permit removal of the upper porous support from the column without substantial removal of the resins.

In other embodiments, the resin may be fixed between two semi-porous frits such that the resin beads may be suspended and such that the upper frit may be removed during washing of the resin to remove background compounds.

The volume of the resin in the column may vary but is typically less than the total packed volume of the column between the lower porous support and the upper porous support. Preferably, the volume of the resin in the column is no greater than 50%, more preferably no greater than 40%, even more preferably no greater than 30%, still more preferably no greater than 25%, and still more preferably no greater than 20% of the total packed volume of the column between the lower porous support and the upper porous support.

SEC columns can be either large or small. In some embodiments, the column contains an agarose/sepharose slurry. In exemplary embodiments, columns may be single use for individual patient samples. For example, purification of the microparticles for preparing the microparticle preparation may involve the use of a qEV original Gen2 35 mm columns (Izon labs; Medford MA) packed with commercial grade Sepharose and/or agarose beads for size exclusion chromatography (SEC). The columns may be washed and allowed to equilibrate to room temperature before being loaded with a sample.

In the purification step, water may be used as a mobile phase. In some embodiments, the water may be distilled water, e.g., double distilled water, deionized water, or deionized and distilled water. In some embodiments, a microparticle-containing sample such as plasma, for example, may be added to the column and water or phosphate buffered saline is used as the mobile phase. Smaller size material or soluble proteins not associated with microparticles may remain associated with the column, while other components of the sample such as larger sized microparticles and content are eluted from the column through a series of washing and elution steps. In embodiments where the sample contains plasma, the water washes may be configured such that high abundance proteins are eluted in later time collected fractions from the column while microparticles elude in the early fractions <10 minutes, and smaller materials remain associated with the column. In some embodiments, the use of water, e.g., distilled water, as the elution buffer offers certain advantages, for example higher yield and improved retention of corona proteins associated with microparticles, including host proteins.

Various modifications to the described purification protocol will be readily apparent to one skilled in the art in view of the present disclosure. For example, the number of elution steps may be modified or adjusted to meet specific purposes. The column may be decorated with reagents that assist in microparticle capture, (affinity purifications).

The enrichment process as described above may allow for microparticle enrichment that is easily accessible for downstream microparticle analysis. This purification process may serve to remove excess background protein and lipid from serum, plasma, and other microparticle-containing samples. The purification may be non-denaturing to yields an enriched sample of microparticles, with or without protein inhibitors. The enriched microparticle fraction may be further analyzed for elements of specific origin without the general problem of steric inhibition by lipids and high abundance proteins. Further, the purification allows for bench top methods such as ELISA and magnetic beads, in addition to more sophisticated but high throughput technologies such as protein mass spectrometry and immuno-analysis may be deployed.

Once the microparticle preparations are made, they can be further processed to quantify MAPs associated with the enriched microparticles for, as noted above and described in further detail below, methods of identifying cancer biomarkers, or methods of determining an aspect of a cancer in a subject.

### Analysis of Expression Levels of MAPs for Identification of Cancer Biomarkers

The present disclosure provides methods of identifying cancer biomarkers based on the expression status or expression level of a plurality of MAPs associated with microparticles from cancer and non-cancer subjects.

In certain embodiments, the method may comprise receiving or obtaining quantification data of a plurality of MAPs in the biological sample obtained from at least one cancer cohort comprising a plurality of cancer patients and at least one non-cancer cohort comprising a plurality of non-cancer subjects. The quantification data may be based on a proteomic analysis of MAPs from microparticle preparations from different cohorts of cancer patients and non-cancer subjects.

In certain embodiments, a microparticle preparation may be processed to isolate MAPs and remove non-protein components, such as cell membranes and other lipid components, or non-protein components of a microparticle lumen. By way of example, the microparticles may be subjected to vesicle lysis using standard methods (e.g. urea or guanidinium buffer extractions).

Once MAPs or fragments thereof are isolated from microparticles, they can be quantified using one of various proteomic analysis methods and platforms known in the art. For example, proteomic analysis may be performed by a mass spectrometer (MS). In some embodiments, the MS may be Liquid Chromatography with tandem mass spectrometry (“LC-MS/MS”).

MS quantification data may be analyzed by various methods known in the art. For example, software tools may be used for Data Dependent Acquisition (DDA) spectral library construction and subsequent Data Independent Acquisition (DIA) analysis. The analysis uses raw data as input files and set corresponding parameters based on human database, then perform identification and quantitative analysis. By way of example, identified peptides that satisfy a condition of False Discovery Rate (FDR)<=1% may be used to construct the final spectral library. One or more of Gene Ontology (GO), Clusters of Orthologous Groups of proteins (COG), and Pathway functional annotation analysis may be also performed in above pipeline. MSstats, which core algorithm is linear mixed effect model, may be used to process DIA quantification result data according to the predefined comparison group, and then a significance test may be performed based on the model. Thereafter, differential protein screening may be performed based on, e.g., Fold Change and statistical significance, e.g., p-value or adjusted p-value (q-value) that is adjusted through, e.g., a Benjamini-Hochberg correction or other correction methods. In some embodiments, a protein may be designated as differentially expressed if the calculated fold change of an expression level of a protein between cancer and non-cancer cohorts is greater than about 1.5, greater than about 1.6, greater than about 1.8, greater than about 2, greater than about 2.2, greater than about 2.4, greater than about 2.5, greater than about 2.6, greater than about 2.8, or greater than about 3. greater than about 1.5. In some embodiments, a protein may be designated as significantly differentially expressed if the calculated p-value between an expression level of a protein between cancer and non-cancer cohorts is less than 0.5, less than 0.4, less than 0.3, less than 0.2, less than 0.1, or less than 0.05. In some embodiments, a protein may be designated as significantly differentially expressed if the calculated q-value (which may be, e.g., an adjusted p-value using a Benjamini-Hochberg correction or other correction methods) between an expression level of a protein between cancer and non-cancer cohorts is less than 0.5, less than 0.4, less than 0.3, less than 0.2, less than 0.1, or less than 0.05.

By way of example, a mass spectrometer such as Eclipse™ may be used to acquire mass spectrometry (MS) data from samples, optionally in Data Independent Acquisition (DIA) mode. A statistical software package such as MSstats may be used to apply intra-system error correction and/normalization for each sample. Then based on the predefined comparison groups and the linear mixed effect model, the significance of differentially expressed proteins (DEPs) may be evaluated. Filtration criteria (e.g., Fold change (increase or decrease)>2 and p-value<0.05) may be used to determine significant differential proteins that are then analyze by various methods such as volcano plots.

Principal component analysis (PCA) may also be applied to the analysis of expression levels of microparticle-associated proteins. PCA is a method of dimension reduction that combines multiple variables to a new set of integrated variables, and then selects several (usually 2-3) to represent as much original information as possible, to achieve the purpose of dimension reduction. PCA is mainly used to observe the trend of separation between groups in the experimental model, and whether there are exceptional value points, and reflect the inter- and intra-group variations from the original data.

Analysis of the expression levels of microparticle-associated proteins may allow for identification of cancer biomarkers (biomarker clusters) that can be used for diagnosis of cancer, or determination of prognosis of a test subject with cancer, etc. Certain biomarker clusters may be better suited for diagnostic methods, compared to prognostic methods (or vice versa), or be better for some cancers, than for others, or be suited as a pan cancer biomarker cluster. Accordingly, the expression profiles of multiple biomarker clusters may be analyzed in order to make an accurate diagnosis, determination of prognosis, etc.

Such expression level analysis methods may include comparing the expression level of two or more microparticle-associated proteins from microparticles from the test subject (i.e. the subject from which the microparticles were isolated) with the expression level of the two or more proteins in samples from a plurality (cohort) of non-cancer subjects or cohort of cancer subjects. The expression levels from samples from the plurality of control subjects may be simultaneously obtained with the test subject expression levels or may constitute a set of numerical values stored on a computer or on computer readable medium. In certain embodiments, the control subjects may be of the same sex, disease stage and of a similar age as the test subject. Control subjects may also be of a similar racial background as the test subject, but need not necessarily be the same.

Comparison of the expression levels of the two or more microparticle-associated proteins in samples from the test subject and from a plurality of control subjects may be performed manually or automatically by a computer program. The expression level of the two or more microparticle-associated proteins in the sample from the test subject may be compared individually to the expression level of the two or more microparticle-associated proteins from samples in each control subject, or the expression level of the two or more microparticle-associated proteins in the sample from the test subject may be compared to an average of the expression levels from samples from the plurality of control subjects. In certain embodiments, the values for the expression levels of the two or more proteins in samples from both the test subject and the plurality of control subjects may be transformed. For example, the expression levels may be transformed by taking the logarithm of the value. Moreover, the expression levels may be normalized by, for example, dividing by the median expression level among all of the samples.

In certain embodiments, the expression level of the two or more microparticle-associated proteins in samples from test cohort (e.g., a cohort of cancer patients) may be increased relative to the expression level of the two or more microparticle-associated proteins in samples from a plurality of control cohort (e.g., a cohort of non-cancer subjects). In other embodiments, the expression level of the two or more microparticle-associated proteins in the samples from the test cohort may be decreased relative to the expression level of the two or more microparticle-associated proteins in samples from the control cohort. Typically, an expression level is said to be increased or decreased relative to a second expression level if the difference between the two expression levels is statistically significant. The difference between two levels is considered to be statistically significant if it was unlikely to have occurred by chance. Statistical significance may be measured by any means known in the art, such as, for example, Fisherian statistical hypothesis testing or the Neyman-Pearson lemma. In certain embodiments, the two or more proteins may not be expressed in samples from the plurality of controls but will be expressed in the sample from the test subject. In other embodiments, the two or more proteins may not be expressed in the sample from the test subject but will be expressed in samples from the plurality of controls.

In certain embodiments, a MAP may be designated as a cancer biomarker based on one or more computational analyses, e.g. based on one or more machine leaming-based analyses of the MAP's differential expression between cancer and non-cancer cohorts. Examples of machine learning based analysis includes but are not limited to Receiver operating characteristic (ROC) curve analysis, random forest (RF) modeling, logistical regression modeling, Exhaustive Feature Selection (EFS), and Recursive Feature Elimination (RFE).

In certain embodiments, a MAP may be designated as a cancer biomarker based on an evaluation of a Receiver operating characteristic (ROC) curve derived from the MAP's differential expression data comparing cancer and non-cancer cohorts. ROC curves are constructed based upon the sensitivity and specificity of the protein of interest, and the area under the curved (AUC) of such ROC curves can be compared to the control data (historical or concurrent) and utilized to define the significance of the observations allowing for an evaluation of sensitivity, specificity, positive and negative predictive values of relevance of the protein signal to the disease state. In an ideal situation, a quantitative cutoff would exist that will perfectly distinguish cancer from non-cancer samples. In this ideal situation, the area under the curve (AUC) of the ROC curve may be calculated to be 1. By contrast, a random analyte which has no predictive value may be calculated to have an AUC of 0.5. As such, in a case for example where a ROC curve is generated based differential expression of a protein biomarker between cancer and non-cancer cohorts, a biomarker having an AUC of the ROC curve that is closer to 1 would be considered to have a higher predictive value for distinguishing cancer from non-cancer samples. In some embodiments, a MAP that is differentially expressed between cancer and non-cancer cohorts may be designated as a cancer biomarker if it has an AUC of the ROC curve of greater than about 0.8, greater than about 0.85, greater than about 0.9, greater than about 0.95, greater than about 0.98, greater than about 0.99, or about 1.

A RF model iteratively builds decision trees by selecting random subsets of features and data points. During this process, it calculates the importance of each feature by measuring how much the tree nodes using that feature reduce impurity. Features with higher impurity reduction are considered more important and receives a higher feature importance score, to be selected for inclusion in the final feature set. In certain embodiments, one or more MAPs may be designated as a cancer biomarker based on an RF model evaluating of the differential expression data comparing cancer and non-cancer cohorts of a plurality of MAPs. In certain embodiments, a feature importance score of a given MAP may be based on the MAP's contribution to the Mean Decrease Impurity (Gini importance) of the RF algorithm.

In certain embodiments, one or more MAPs may be designated as a cancer biomarker based on Logistic Regression. Logistic regression is a statistical model used for binary classification tasks, where the outcome variable is categorical and has two possible outcomes, e.g. for classifying cancer vs. non-cancer. The model estimates the probability that a given instance (e.g. a microparticle preparation from a subject suspected of cancer) belongs to a particular category (e.g., cancer vs. non-cancer) based on one or more independent variables, which may be the respective expression level of a plurality of MAPs. Logistic Regression model trained on the differential expression levels of a plurality of MAPs may be used to identify MAPs that provide a high degree of accuracy for predicting cancer vs. non-cancer. A given model may be validated with cross validation, which is used to assess how well a model will generalize to an independent dataset. In cross validation, the dataset is divided into multiple subsets, or “folds”. A given fold is designated as a validation set and the remaining folds are designated as a training set for training a model. For example, if the dataset is divided up into 5 folds, then the model may be trained on the 4 folds of the training set, then tested against the remaining fold that is used as a validation set. In certain embodiments, the dataset may be divided up in to between 3 and 10 folds, 3 folds, 4 folds, 5 folds, 6 folds, 7 folds, 8 folds, 9 folds, or 10 folds. This process may be repeated several times, each time with a different fold designated as the validation set. In some embodiments, the cross validation may be stratified. In stratified cross validation, when splitting the data into folds, each fold is made to preserve the same proportion of the target classes as the original dataset. For example, if the dataset contains 80% cancer samples and 20% non-cancer samples, each fold may also contain roughly the same proportions of these classes.

In certain embodiments, methods for identifying cancer biomarker may include identifying a panel of biomarkers, e.g. identifying a panel of a predefined number of biomarkers, which may be referred to as a “multiplex”, whose combined expression levels are especially predictive of a cancer in a subject. In some embodiments, a multiplex may comprise between 2 and 20 biomarkers, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, or 20 biomarkers. As used herein, a multiplex consisting of 3 biomarkers may be referred to herein as a “3plex”, a multiplex consisting of 4 biomarkers may be referred to herein as a “4plex”, and so on.

In certain embodiments, multiplexes of MAPs predictive of a cancer may be identified computationally using Recursive Feature Elimination (RFE). RFE systematically removes less important features by recursively training a model and ranking features based on their contribution to model performance. The process continues until the desired number of features remains or until a specified performance metric is optimized. In some embodiments, a plurality of MAPs may be evaluated with an RFE algorithm to identify a predefined number, which may be referred to as a multiplex, of MAPs that most contribute to model performance. An RFE algorithm may identify different multiplexes from a given plurality of MAPs.

In certain embodiments, multiplexes of MAPs predictive of a cancer may be identified using Exhaustive Feature Selection (EFS).

Exhaustive feature selection may be used to identify the most predictive combination of biomarkers for distinguishing between cancer and non-cancer cases based on their expression levels. Starting from a dataset with a large set of biomarkers and corresponding expression levels for both cancer and non-cancer samples, to determine the best combination of biomarkers for predicting cancer, an EFS algorithm may systematically evaluate all possible subsets (e.g., all possible 3plexes of a set of biomarkers) a from the larger set of biomarkers. For each subset, a predictive model, such as logistic regression or a decision tree, may be trained and evaluated using performance metrics tailored to binary classification, such as the area under the receiver operating characteristic (ROC) curve. The subsets that yield the highest predictive performance may then be selected as a set of optimal multiplexes for distinguishing between cancer and non-cancer samples based on the expression levels of the constituent biomarkers.

In some embodiments, two or more of the above-noted analytical and machine learning method may be combined to identify cancer biomarker multiplexes. By way of examples, the differential expression data of a plurality of MAPs obtained from cancer and non-cancer cohorts may be analyzed to select a first subset of MAPs to be designated as candidate biomarkers based on fold change and p-value or q-value, for example by eliminating MAPs having less than a minimum fold change threshold value and eliminating MAPs having a p-value or q-value that is more than a maximum threshold value. The first subset of candidate biomarkers may then be further analyzed with ROC curve AUC analysis, a RF model, or Logistic Regression to generate a further narrowed second subset of MAPs designated as cancer biomarkers. This second set of cancer biomarkers may then be analyzed with RFE or EFS to identify multiplexes of cancer biomarkers that are especially predictive.

In some embodiments, the method of identifying cancer biomarkers may comprise identifying cancer biomarkers based on ROC curve AUC analysis, then identifying cancer biomarker multiplexes with RFE.

In some embodiments, the method of identifying cancer biomarkers may comprise identifying cancer biomarkers based on RF analysis, then identifying cancer biomarker multiplexes with RFE.

In some embodiments, the method of identifying cancer biomarkers may comprise identifying cancer biomarkers based on ROC curve AUC analysis, then identifying cancer biomarker multiplexes with EFS.

In some embodiments, the method of identifying cancer biomarkers may comprise identifying cancer biomarkers based on RF analysis, then identifying cancer biomarker multiplexes with EFS.

In some cases, when a plurality of predictive multiplexes are identified, some individual biomarkers may be overrepresented within the set of identified predictive multiplexes, and thus represent biomarkers that are particular useful in predicting cancer as part of a multiplex. Such biomarkers may be referred to herein as “key” biomarkers. In some embodiments, the method of identifying cancer biomarkers may comprise identifying a plurality of cancer biomarker multiplexes, then identifying one or more key biomarkers based on the cancer biomarker multiplexes.

### Analysis of Expression Levels of MAPs for Cancer Diagnosis

The present disclosure provides methods of analyzing microparticles to determine the respective expression status or expression level of a plurality of MAPs for detection, determination, diagnosis or prognostication of one or more aspects of a cancer in a subject. The plurality of MAPs may be cancer biomarkers identified, e.g., by methods described herein.

The expression level of a protein may include an absolute amount of a protein from a microparticle, or it may simply refer to the presence or absence of a protein in a sample. The expression level may also be a relative amount compared to microparticles from a different condition (e.g. microparticles derived from cancer patients compared with those derived from non-cancer subjects or a different timepoint in a same patient). The expression level may also be compared to a reference standard. The expression level of the microparticle-associated protein may be detected by any methods known to one of skill in the art, which may in certain embodiments be an immunoassay (see, for example: Coligan et al, Unit 9, Current Protocols in Immunology, Wiley Interscience, 1994). Examples of immunoassays include: antibody detection, immunohistochemistry (Microscopy, Immunohisto chemistry and Antigen Retrieval Methods for Light and Electron Microscopy, M. A. Hayat (Author), Kluwer Academic Publishers, 2002; Brown C: “Antigen retrieval methods for immunohistochemistry,” Toxicol Pathol 1998; 26(6): 830-1), ELISA (Onorato et al., “Immunohistochemical and ELISA assays for biomarkers of oxidative stress in aging and disease,” Ann NY Acad Sci 1998 20; 854: 277-90), Western blotting (Laemmeli UK: “Cleavage of structural proteins during the assembly of the head of a bacteriophage T4,” Nature 1970; 227: 680-685; Egger & Bienz, “Protein (western) blotting”, Mol Biotechnol 1994; 1(3): 289-305), and antibody microarray (Huang, “Detection of multiple proteins in an antibody-based protein microarray system,” Immunol Methods 2001 1; 255 (1-2): 1-13) as well as novel affinity readouts of protein presence using Proximity extension assay protein profiling of liquid biopsy samples using commercial or custom-made immunoaffiinty readouts (Olink Proteomics AB, Uppsala, Sweden; Alamar, Inc.). Other examples include a proximity ligation assay using a selected antibody with nucleic acid tag that can be amplified by primers for detection of small protein quantities. In certain embodiment, the expression of protein may be quantified using an affinity capture assay that utilizes a capture agent where, said capture agent is selected from the group consisting of an antibody or fragment thereof, a nucleic acid-based protein binding reagent (e.g., a, and a small molecule). Other protein quantification methods include mass spectrometry, aptamer-based detection, single-molecule array assay (SIMO A), a proximity extension assay, protein identification by short epitope mapping, and protein sequencing.

Various approaches may be used in preparation for detecting the expression levels of the MAPs for detection, determination, diagnosis or prognostication of one or more aspects of a cancer in a subject. In one approach, the proteins may be dissociated from microparticles. For example, the microparticles may be lysed, and the proteins in the microparticles may be extracted, precipitated, and reconstituted for analysis. In another approach, the microparticles are kept intact so that the protein remains associated, and the microparticles are attached to a column, resin, or bead. The reconstituted protein or the microparticles attached to a column, resin, or bead are used in the detection step. For example, the reconstituted protein or the microparticles attached to a column, resin, or bead are contacted with an antibody specific to the protein biomarker.

In certain embodiments, detecting the expression level includes detecting binding of the protein to an antibody specific to the protein. Antibodies may be monoclonal or polyclonal, included fragments, and they may be obtained from a commercial source or generated for use in the methods described herein. Methods for producing and evaluating antibodies are well known in the art, see, e.g., Coligan, (1997) Current Protocols in Immunology, John Wiley & Sons, Inc; and Harlow and Lane (1989) Antibodies: A Laboratory Manual, Cold Spring Harbor Press, NY (“Harlow and Lane”).

The antibody may be covalently bound to a bead or fixed on a solid surface, such as glass, plastic, or silicon chip. Typically, microparticle-associated proteins are contacted with an antibody specific to at least one protein biomarker. Any protein biomarker present in the sample will bind to the specific antibody. The mixture is washed, and the antibody-protein biomarker complexes can be detected.

This detection can be achieved by contacting the washed antibody-protein biomarker complexes with a detection reagent. This detection reagent may be, for example, a secondary antibody which is labeled with a detectable label. Exemplary detectable labels include magnetic beads (e.g., DYNABEADS™), fluorescent dyes, radiolabels, enzymes (e.g., horseradish peroxide, alkaline phosphatase, and others commonly used in ELISA), and colorimetric labels such as colloidal gold, colored glass, or plastic beads.

Methods for measuring the amount or presence of antibody-biomarker complexes may include, for example, detection of fluorescence, luminescence, chemiluminescence, absorbance, reflectance, transmittance, birefringence, or refractive index (e.g., surface plasmon resonance, ellipsometry, a resonant mirror method, a grating coupler waveguide method, or interferometry). Optical methods include microscopy (both confocal and non-confocal), imaging methods, dynamic light scattering, fluorescent NanoSight Tracking Analysis (NanoSight Ltd., Wiltshire UK) and non-imaging methods. Electrochemical methods include voltametry and amperometry methods. Radio frequency methods include multipolar resonance spectroscopy. Methods for performing these assays are readily known in the art. Useful assays may include, for example, an enzyme immune assay (EIA) such as enzyme-linked immunosorbent assay (ELISA), a radioimmune assay (RIA), a Western blot assay, immuno-PCR using proximal ligation assay (PLA) or proximity extension assays (PEA) in the form of pre-conjugated kits or customized designed protein detecting kits (Life Technologies, Carlsbad, CA, Olink Bioscience, Uppsala, Sweden) and high sensitivity protein immunoassay (Life Technologies ProQuantum). or slot blot assay. These methods are also described in, e.g., Nature Scientific Reports volume 11 Sun and Meckes (2021); as well as in Methods in Cell Biology: Antibodies in Cell Biology, volume 37 (Asai, ed. 1993); Basic and Clinical Immunology (Stites & Terr, eds., 7th ed. 1991); and Harlow & Lane, supra. In preferred embodiments, detecting binding of the protein biomarker to an antibody specific to the biomarker includes detecting fluorescence or other methods of quantification.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, the volume of solution, concentrations, and the like. Usually the assays will be carried out at ambient temperature, although they can be conducted over a range of temperatures, such as 10° C. to 40° C.

Immunoassays may also be used to determine the presence or absence of a microparticle-associated protein as well as the quantity of the microparticle-associated protein. The amount of an antibody-biomarker complex can be determined by comparing to a standard. A standard may be, for example, a known compound or another protein known to be present in a sample. As noted above, the test amount of marker need not be measured in absolute units, as long as the unit of measurement can be compared to a reference value.

In some embodiments, the methods of detecting the expression levels of the MAPs involve detecting the expression level of clusters or panels of a plurality of MAPs. Detecting the expression level of multiple protein biomarkers can be achieved, for example, with a protein microarray such as an antibody microarray. The production of such microarrays can be carried out essentially as described in Schweitzer & Kingsmore, “Measuring proteins on microarrays,” Curr Opin Biotechnol 2002; 13(1): 14-9; Avseenko et al., “Immobilization of proteins in immunochemical microarrays fabricated by electrospray deposition,” Anal Chem 2001 15; 73(24): 6047-52; Huang, “Detection of multiple proteins in an antibody-based protein microarray system,” Immunol Methods 2001 1; 255 (1-2): 1-13. In general, protein microarrays may be produced essentially as described in Schena et al., “Parallel human genome analysis: Microarray-based expression monitoring of 1000 genes,” Proc. Natl. Sci. USA (1996) 93, 10614-10619; U.S. Pat. Nos. 6,291,170 and 5,807,522 (see above); U.S. Pat. No. 6,037,186 (Stimpson, inventor) “Parallel production of high density arrays,” WO 99/13313 (Genovations Inc (US), applicant) “Method of making high density arrays,” WO 02/05945 (Max Delbruck Center for Molecular Medicine (Germany), applicant) “Method for producing microarray chips with nucleic acids, proteins or other test substrates.”

Protein or antibody microarray hybridization may be carried out as described in Ekins et al. J Pharm Biomed Anal 1989. 7: 155; Ekins and Chu, Clin Chem 1991. 37: 1955; Ekins and Chu, Trends in Biotechnology, 1999, 17, 217-218; MacBeath and Schreiber, Science 2000; 289(5485): p. 1760-1763.

In certain embodiments, once two or more biomarkers, e.g., in a microparticle preparation from a subject, have been quantified, e.g., using one or more of the methods noted above, a dataset comprising respective quantitative measures of the two or more biomarkers may be used to determine or predict an aspect of a cancer in the subject, e.g., determine whether or not the subject has the cancer.

It will be appreciated that any number of biomarkers may be used for the analyses provided herein. The two or more biomarkers may be between 2 and 20 biomarkers, between 4 and 10 biomarkers, between 2 and 8 biomarkers, between 3 and 5 biomarkers, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, 20 biomarkers, or more than 20 biomarkers.

In some embodiments, a dataset containing quantitative measures of two or more biomarkers may be classified, for example, into cancer or non-cancer categories, using a trained algorithm. This algorithm, trained with reference data, may act as a classifier to identify patterns in the data for classification purposes. The present disclosure includes any known pattern recognition methods known in the art, such as logistic regression, random forest, support vector machine (SVM), k-nearest neighbor, neural network, XGBoost, lightGBM, gradient boosting classifier, and AdaBoost classifier. Additional pattern recognition algorithms are also contemplated by these methods.

The training of the algorithm typically involves using a labeled reference dataset, where the outcomes (e.g., cancer or non-cancer) are already known. This dataset may be divided into a training set and a validation set. The training set may be used to teach the algorithm by allowing it to identify patterns and correlations between the biomarkers and the known outcomes. Various techniques, such as cross-validation and hyperparameter tuning, may be employed to optimize the model's performance. The validation set may then be used to evaluate the algorithm's accuracy and generalization capability. In some embodiments, the algorithm may become more proficient at classifying new, unseen data (that is, data not presented during training) based on the patterns it has learned by iteratively adjusting the model and testing its predictions. In some embodiments, the trained algorithm (e.g., a classifier) may be predictive of cancer states in a subject based on unseen data at an accuracy of at least 80%, at least 85%, at least 90%, at least 91%, at least 92%, at least 91%, at least 93%, at least 94%, at least 95%, at least 96%, at least 97%, at least 98%, at least 99%, more than 99%, or 100%. Accuracy of a trained algorithm may be calculated, e.g., as a ratio of the number of instances correctly predicted by the classifier, to the total number of instances tested with a validation set.

The training process of the algorithm may involve adjusting beta coefficients to optimize its predictive accuracy. This adjustment is typically performed through iterative algorithms. For example, during each iteration, the algorithm may calculate prediction error by comparing the predicted outcomes to the actual outcomes in the training dataset. It may then update the beta coefficients in a direction that reduces this error. For example, in gradient descent, the weights may be adjusted in proportion to the negative gradient of the error with respect to each weight, effectively minimizing the error function. This process is repeated until the algorithm converges to a set of weights that result in maximally accurate predictions. Regularization techniques may also be applied to prevent overfitting, ensuring that the algorithm performs well on both training and unseen data.

In some embodiments, the training data used to train the algorithm may be based on, or include the same data (e.g. MAP quantification data from mass spectroscopy) that was used to identify the biomarkers used for the classification. In some embodiments, aspects of the results of the training process used to identify the biomarker, e.g., classification rules and beta coefficients, may be applied to the algorithm used to perform classifications (e.g., cancer vs. non-cancer) with new and unseen data.

In certain embodiments, the trained algorithm may be a logistic regression algorithm. In the context of logistic regression, the training process may involve adjusting the beta coefficients to best fit the model to the training data. This process may start with initializing the weights, which may be to small random values. The algorithm then makes predictions on the training data using these initial weights, applying the logistic function to compute the probability that each instance belongs to the positive class (e.g., cancer). The training process may include iterations of making predictions, computing a loss, and updating the weights, until the weights converge to values that minimize a loss function.

In certain embodiments, a final trained logistic regression algorithm with its beta coefficients may be a mathematical model that predicts the probability of a binary outcome (e.g., cancer vs. non-cancer) based on the input features (quantitative measures of biomarkers). The model is typically represented by the logistic function applied to a linear combination of the input features and their corresponding weights.

For example, a logistic regression model for classifying cancer vs. non-cancer based on the quantitative measures of three biomarkers may be expressed as:

$$Probability ( target = ‘ Cancer ’ ) = 1 / 1 + exp \hat{} - ( beta 0 + beta 1 * Protein 1 + beta 2 * Protein 2 + beta 3 * Protein 3 )$$

where Protein1, Protein2 and Protein3 are the quantitative measures, respectively of each biomarker, beta 0 is an intercept or bias coefficient, beta 1 is a beta coefficient for Protein1, beta2 is a beta coefficient for Protein2, and beta3 is a beta coefficient for Protein3.

The probability outcome may be set to provide a binary outcome where, e.g., probability >0.5 is cancer and probability <=0.5 is normal (non-cancer).

In some embodiments, the analysis of quantitative measures of biomarkers may involve use of a computer system comprising a processor and a memory coupled to the processor. The memory may store a module comprising test data for a sample from a subject, or test data for a plurality of samples, respectively, from each of a plurality of subjects. The module may further comprise a trained algorithm as described above (e.g., a classifier) configured to classify the subject or plurality of subjects as having a cancer or not having the cancer based on the test data. The module may further comprise computer executable instructions for implementing the classifier on the test data. In some embodiments, the computer system may be implemented as a distributed cloud network, comprises a plurality of interconnected nodes, each node comprising a processor and a memory operably connected to the processor, that are configured to collaboratively execute computational tasks. In some embodiments, the computer system may be embodied as a standalone laptop or desktop computer, each comprising a processor and a memory operably connected the processor, as well as input/output interfaces for user interaction and peripheral connectivity.

### Types of Cancers

Cancer, with respect to methods disclosed in the present application may include, but are not limited, to acute lymphoblastic leukemia, acute myeloid leukemia, adrenocortical carcinoma, aids-related cancers, aids-related lymphoma, anal cancer, appendix cancer, basal cell carcinoma, extrahepatic bile duct cancer, bladder cancer, bone cancer, osteosarcoma and malignant fibrous histiocytoma, adult tumor, central nervous system atypical teratoid/rhabdoid tumor, brain cancer, astrocytomas, supratentorial primitive neuroectodermal tumors and pineoblastoma, brain tumor, spinal cancer. spinal cord tumor, breast cancer, bronchial tumors, burkitt lymphoma, primary central nervous system lymphoma, cervical cancer, chordoma, chronic lymphocytic leukemia, chronic myelogenous leukemia, chronic myeloproliferative disorder, colon cancer, colorectal cancer, abdominal cancer, craniopharyngioma, endometrial cancer, ependymoblastoma, ependymoma, esophageal cancer, Ewing sarcoma family of tumors, extracranial germ cell tumor, extragonadal germ cell tumor, gallbladder cancer, gastric (stomach) cancer, gastrointestinal carcinoid tumor, gastrointestinal stromal cell tumor (gist), extragonadal germ cell tumor, ovarian germ cell tumor, gestational trophoblastic tumor, glioma, hairy cell leukemia, head and neck cancer, hepatocellular (liver) cancer, adult Hodgkin lymphoma, hypopharyngeal cancer, intraocular melanoma, islet cell tumors (endocrine pancreas), Kaposi sarcoma, renal cancer, Langerhans cell histiocytosis, laryngeal cancer, chronic lymphocytic leukemia, chronic myelogenous leukemia, hairy cell leukemia, lip cancer, oral cavity cancer, liver cancer, lung cancer (e.g., non-small cell lung cancer or small cell lung cancer), non-Hodgkin lymphoma, primary central nervous system lymphoma, Waldenstrom macroglobulinemia, malignant fibrous histiocytoma of bone and osteosarcoma, medulloblastoma, medulloepithelioma, melanoma, Merkel cell carcinoma, malignant mesothelioma, metastatic squamous neck cancer with occult primary, multiple endocrine neoplasia syndrome, multiple myeloma/plasma cell neoplasm, mycosis fungoides, myelodysplasia syndromes, myelodysplastic/myeloproliferative neoplasms, chronic myelogenous leukemia, multiple myeloma, chronic myeloproliferative disorders, nasal cavity cancer, paranasal sinus cancer, nasopharyngeal cancer, neuroblastoma, oropharyngeal cancer, ovarian cancer, pancreatic cancer, parathyroid cancer, penile cancer, pharyngeal cancer, pituitary tumor, plasma cell neoplasm/multiple myeloma, pleuropulmonary blastoma, prostate cancer, rectal cancer, respiratory tract carcinoma, retinoblastoma, rhabdomyosarcoma, salivary gland cancer, soft tissue sarcoma, uterine sarcoma, Sezary syndrome, skin cancer (e.g., nonmelanoma skin cancer or melanoma), Merkel cell skin carcinoma, small intestine cancer, soft tissue sarcoma, squamous cell carcinoma, cutaneous t-cell lymphoma, testicular cancer, throat cancer, thymoma carcinoma, thymic carcinoma, thyroid cancer, gestational trophoblastic tumor, transitional cell cancer of ureter and renal pelvis, urethral cancer, endometrial uterine cancer, uterine sarcoma, vaginal cancer, vulvar cancer, fallopian cancer, peritoneal cancer, and Wilms' tumor.

In some embodiments, the cancer is a solid tumor. In some embodiments, the solid tumor is a colorectal cancer, a breast cancer, an ovarian cancer, a uterine cancer, a fallopian cancer, a lung cancer (e.g., a non-small cell lung cancer), a brain cancer, a spinal cancer, a head and neck cancer, a pancreatic cancer, a prostate cancer, a renal cancer, a gastric cancer, a sarcoma, a liver cancer, an abdominal cancer, a peritoneal carcinoma, or a bladder cancer.

Cancers may be grouped into stages, ranging from Stage 0 to Stage 4: Stage 0 (Carcinoma in situ—Cancer is in its earliest stage, has not spread, and is usually highly treatable); Stage 1 (Localized Cancer—Cancer is small and localized to one area; often referred to as early-stage cancer); Stage 2 and 3 (Regional Spread—Cancer grown larger and may have spread to nearby lymph nodes or tissues but not to distant parts of the body); Stage 4 (Distant Spread—Cancer has spread to distant parts of the body; often referred to as advanced or metastatic cancer). In some embodiments, the method of determining an aspect of a cancer in a subject may be diagnosing, determining, or prognosticating the presence in a subject of a stage 0 cancer, a stage 1 cancer, a stage 2 cancer, a stage 3 cancer, a stage 4 cancer, or combinations thereof.

### Summary of Cancer Biomarkers

Below is a brief summary of each table referenced in the detailed description, examples and claims:

Table 2.1 provides pan cancer biomarkers.

Table 2.2 provides an exemplary multiplex of pan cancer biomarkers.

Table 3.1 provides breast cancer biomarkers.

Table 4.1 provides colorectal cancer biomarkers.

Table 5.1 provides lung cancer biomarkers.

Table 6.1 provides ovarian cancer biomarkers.

Table 7.1 provides ovarian cancer biomarkers.

Table 7.2 provides ovarian cancer biomarkers.

Table 7.3 provides ovarian cancer biomarkers.

Table 8.2 provides pan cancer biomarkers.

Table 8.3 provides pan cancer biomarker 3plexes based on the biomarkers of Table 8.2.

Table 8.4 provides the most common biomarkers in the pan-cancer biomarker 3plexes of Table 8.3.

Table 9.2 provides ovarian cancer biomarkers.

Table 9.3 provides ovarian cancer biomarker 3plexes based on the biomarkers of Table 9.2.

Table 9.4 provides the most common biomarkers in the ovarian cancer biomarker 3plexes of Table 9.3.

Table 9.5 provides lung cancer biomarkers.

Table 9.6 provides lung cancer biomarker 3plexes based on the biomarkers of Table 9.5.

Table 9.7 provides the most common biomarkers in the lung cancer biomarker 3plexes of Table 9.6.

Table 9.8 provides colorectal cancer biomarkers.

Table 9.9 provides colorectal cancer biomarker 3plexes based on the biomarkers of Table 9.8.

Table 9.10 provides the most common biomarkers in the colorectal cancer biomarker 3plexes of Table 9.9.

Table 9.11 provides breast cancer biomarkers.

Table 9.12 provides breast cancer 3plexes based on the biomarkers of Table 9.11.

Table 9.13 provides the most common biomarkers in the breast cancer biomarker 3plexes of Table 9.12.

Table 9.14 provides pan cancer biomarkers (from Table 8.2) that are significantly differentially expressed across all tested comparisons (consensus pan cancer biomarkers).

Table 9.15 provides consensus pan cancer biomarker 3plexes based on the biomarkers of Table 9.14.

Table 9.16 provides the most common biomarkers in the consensus pan cancer biomarker 3plexes of Table 9.15.

The contents of each table are provided for in the Examples section, but is incorporated by reference herein, in the Detailed Description.

### Pan Cancer Biomarkers

During development of the present disclosure, numerous MAPs were determined to be differentially expressed in samples from cohorts of subjects having one of a plurality of cancer types compared to samples from non-cancer subjects. These differentially expressed MAPs were further analyzed to identify cancer biomarkers and multiplexes of cancer biomarkers that were determined to be predictive of cancer states in a subject. In certain embodiments, the cancer biomarkers or multiplexes thereof may be predictive of cancer states at an accuracy of at least 80%, at least 85%, at least 90%, at least 91%, at least 92%, at least 91%, at least 93%, at least 94%, at least 95%, at least 96%, at least 97%, at least 98%, at least 99%, more than 99%, or 100%. In some embodiments, an accuracy of a biomarker or a multiplex of biomarkers may be calculated, e.g., as a ratio of the number of instances correctly predicted by a trained classifier based on a quantitative measure of the biomarker or multiplex or biomarkers, to the total number of instances tested with a validation set. The plurality of cancer types studied included a wide range of cancers: ovarian cancer, colorectal cancer, breast cancer, and non-small cell lung cancer. As such, the cancer biomarkers identified based on a combined analysis of all cancer types are referred to herein as “pan cancer biomarkers”. As such the present disclosure provides microparticle-associated proteins (MAPs) that may be useful as pan cancer biomarkers, as well as multiplexes thereof, in detection or prognostication of one or more aspects of a cancer in a subject. In some embodiments, the cancer may be any one of ovarian cancer, colorectal cancer, breast cancer, and non-small cell lung cancer.

In certain embodiments, methods of determining an aspect of a cancer in a subject (e.g., diagnosing or prognosticating the presence of a cancer) may include providing a microparticle preparation from a biological sample from the subject, and quantifying two or more proteins in the fraction, wherein the two or more proteins are selected from Tables 2.1, 2.2, 3.1, 4.1, 5.1, 6.1, 7.1-73, 8.2-8.4, or 9.2-9.16.

In certain embodiments, the two or more proteins are biomarkers selected from Tables 2.1 or 8.2. In certain embodiments, the two or more proteins comprise a multiplex selected from Table 2.2 or Table 8.3. In certain embodiments, the multiplex comprises at least one protein selected from Table 8.4. In certain embodiments, the multiplex comprises one or both of CO3 and PROS.

In certain embodiments, the two or more proteins are biomarkers selected from Table 9.14. In certain embodiments, the two or more proteins comprise a multiplex selected from Tables 9.15. In certain embodiments, the multiplex comprises at least one protein selected from Table 9.16. In certain embodiments, the multiplex comprises one or both of CO3 and PROS.

### Cancer-Type Specific Biomarkers

During development of the present disclosure, numerous MAPs were determined to be differentially expressed in samples from breast cancer patients compared to samples from non-cancer subjects. These differentially expressed MAPs were further analyzed for example using the machine learning and other computational methods provided herein to identify breast cancer biomarkers and multiplexes of breast cancer biomarkers that were determined to be predictive of a breast cancer state in a subject. In certain embodiments, the breast cancer biomarkers or multiplexes thereof may be predictive of cancer states at an accuracy of at least 80%, at least 85%, at least 90%, at least 91%, at least 92%, at least 91%, at least 93%, at least 94%, at least 95%, at least 96%, at least 97%, at least 98%, at least 99%, more than 99%, or 100%. In some embodiments, an accuracy of a biomarker or a multiplex of biomarkers may be calculated, e.g., as a ratio of the number of instances correctly predicted by a trained classifier based on a quantitative measure of the biomarker or multiplex or biomarkers, to the total number of instances tested with a validation set. As such the present disclosure provides microparticle-associated proteins (MAPs) that may be useful as breast cancer biomarkers, as well as multiplexes thereof, in detection or prognostication of one or more aspects of breast cancer in a subject. In certain embodiments, the two or more proteins are biomarkers selected from Table 3.1 or Table 9.11. In certain embodiments, the two or more proteins comprise a multiplex selected from Tables 9.12. In certain embodiments, the multiplex comprises at least one protein selected from Table 9.13. In certain embodiments, the multiplex comprises one, two, or three out of PHLD, FIBA, FIBG, and HEP2.

During development of the present disclosure, numerous MAPs were determined to be differentially expressed in samples from lung cancer patients compared to samples from non-cancer subjects. These differentially expressed MAPs were further analyzed for example using the machine learning and other computational methods provided herein to identify lung cancer biomarkers and multiplexes of lung cancer biomarkers that were determined to be predictive of a lung cancer state in a subject. In certain embodiments, the lung cancer biomarkers or multiplexes thereof may be predictive of cancer states at an accuracy of at least 80%, at least 85%, at least 90%, at least 91%, at least 92%, at least 91%, at least 93%, at least 94%, at least 95%, at least 96%, at least 97%, at least 98%, at least 99%, more than 99%, or 100%. In some embodiments, an accuracy of a biomarker or a multiplex of biomarkers may be calculated, e.g., as a ratio of the number of instances correctly predicted by a trained classifier based on a quantitative measure of the biomarker or multiplex or biomarkers, to the total number of instances tested with a validation set. As such the present disclosure provides microparticle-associated proteins (MAPs) that may be useful as lung cancer biomarkers, as well as multiplexes thereof, in detection or prognostication of one or more aspects of lung cancer in a subject. In certain embodiments, the two or more proteins are biomarkers selected from Table 5.1 or Table 9.5. In certain embodiments, the two or more proteins comprise a multiplex selected from Tables 9.6. In certain embodiments, the muliplex comprises at least one protein selected from Table 9.7. In certain embodiments, the multiplex comprises one, two, or three proteins out of HEP2, C4PBP, and PROS.

During development of the present disclosure, numerous MAPs were determined to be differentially expressed in samples from colorectal cancer patients compared to samples from non-cancer subjects. These differentially expressed MAPs were further analyzed for example using the machine learning and other computational methods provided herein to identify colorectal cancer biomarkers and multiplexes of colorectal cancer biomarkers that were determined to be predictive of a colorectal cancer state in a subject. In certain embodiments, the colorectal cancer biomarkers or multiplexes thereof may be predictive of cancer states at an accuracy of at least 80%, at least 85%, at least 90%, at least 91%, at least 92%, at least 91%, at least 93%, at least 94%, at least 95%, at least 96%, at least 97%, at least 98%, at least 99%, more than 99%, or 100%. In some embodiments, an accuracy of a biomarker or a multiplex of biomarkers may be calculated, e.g., as a ratio of the number of instances correctly predicted by a trained classifier based on a quantitative measure of the biomarker or multiplex or biomarkers, to the total number of instances tested with a validation set. As such the present disclosure provides microparticle-associated proteins (MAPs) that may be useful as colorectal cancer biomarkers, as well as multiplexes thereof, in detection or prognostication of one or more aspects of colorectal cancer in a subject. In certain embodiments, the two or more proteins are biomarkers selected from Table 4.1 or Table 9.8 In certain embodiments, the two or more proteins comprise a multiplex selected from Tables 9.9. In certain embodiments, the multiplex comprises at least one protein selected from Table 9.10. In certain embodiments, the multiplex comprises one, two, or three proteins out of CIQB, APOA4, PROS, and ECM1.

During development of the present disclosure, numerous MAPs were determined to be differentially expressed in samples from ovarian cancer patients compared to samples from non-cancer subjects. These differentially expressed MAPs were further analyzed for example using the machine learning and other computational methods provided herein to identify ovarian cancer biomarkers and multiplexes of ovarian cancer biomarkers that were determined to be predictive of an ovarian cancer state in a subject. In certain embodiments, the ovarian cancer biomarkers or multiplexes thereof may be predictive of cancer states at an accuracy of at least 80%, at least 85%, at least 90%, at least 91%, at least 92%, at least 91%, at least 93%, at least 94%, at least 95%, at least 96%, at least 97%, at least 98%, at least 99%, more than 99%, or 100%. In some embodiments, an accuracy of a biomarker or a multiplex of biomarkers may be calculated, e.g., as a ratio of the number of instances correctly predicted by a trained classifier based on a quantitative measure of the biomarker or multiplex or biomarkers, to the total number of instances tested with a validation set. As such the present disclosure provides microparticle-associated proteins (MAPs) that may be useful as ovarian cancer biomarkers, as well as multiplexes thereof, in detection or prognostication of one or more aspects of ovarian cancer in a subject. In certain embodiments, the two or more proteins are biomarkers selected from Tables 6.1, 7.1, 7.2, 7.3, or 9.2. In certain embodiments, the two or more proteins comprise a multiplex selected from Tables 9.3. In certain embodiments, the multiplex comprises at least one protein selected from Table 9.4. In certain embodiments, the multiplex comprises one, two, or three proteins out of C4BPB, APOA4, PCGBP, PHLD, HABP2, and FIBA.

### Immunomodulation Biomarkers

During development of the present disclosure, it was found that certain MAPs determined to be differentially expressed in samples from cancer patients compared to samples from non-cancer subjects included proteins involved in the immune response. Examples of such immunomodulatory biomarkers include colony stimulating factor 1 receptor, an antigen presenting cell marker, and Fibrinogen-like protein 1, a tumor immune suppressor, as well as innate immunity proteins such as Complement Factor H, Complement Component 1 Subcomponent S, an Complement Component 1q These differentially expressed MAPs may be used individually or in combination as biomarkers predictive of an immunomodulated state (e.g. cancer-based immunosuppression) of a subject. These biomarkers may be predictive at an accuracy of at least 80%, at least 85%, at least 90%, at least 91%, at least 92%, at least 91%, at least 93%, at least 94%, at least 95%, at least 96%, at least 97%, at least 98%, at least 99%, more than 99%, or 100%. In some embodiments, an accuracy of a biomarker or a multiplex of biomarkers may be calculated, e.g., as a ratio of the number of instances correctly predicted by a trained classifier based on a quantitative measure of the biomarker or multiplex or biomarkers, to the total number of instances tested with a validation set.

Such immunomodulatory biomarkers may be useful in determining presence of a host immunomodulated environment in a subject, for example cancer-induced immunomodulated environments.

Accordingly, in certain aspects, the present disclosure includes methods of determining presence of a cancer-induced host immunomodulated environment in a subject. In certain embodiments, a method according to the disclosure may comprise providing a microparticle preparation from a biological fluid sample from a subject; quantifying two or more proteins a the microparticle preparation, wherein the two or more proteins include at least one immunomodulatory biomarker; and based on the quantification of the two or more proteins, determining the presence of the cancer-induced immunomodulation in the subject.

Also, in certain aspects, the present disclosure includes methods of treating cancer in a subject through first detecting cancer-induced immunomodulation. In certain embodiments, a method according to the disclosure may comprise providing a microparticle preparation from a biological sample from the subject; quantifying two or more proteins in the microparticle preparation, wherein the two or more proteins include at least one immunomodulatory biomarker: determining presence of cancer-induced immunomnodulation in the subject based on the quantification of the two or more proteins; and administering an effective amount of an immune response modulator to the subject based on the determination of the presence of cancer-induced inmunomodulation in the subject, thereby treating the cancer.

In certain embodiments, cancer-induced immunomodulation may be cancer-induced immunosuppression.

In certain embodiments, the immune response modulator may be a checkpoint inhibitors, which may include PD-1 inhibitors such as pembrolizumab and nivolumab, and PD-L1 inhibitors such as atezolizumab and durvalumab. In certain embodiments, the immune response modulator may be a cytokine, such as IL-2 and interferon-alpha. Other examples of immune response modulators include CAR-T cell therapies such as tisagenlecleucel and axicabtagene ciloleucel, and monoclonal antibodies such as rituximab (targeting CD20) and trastuzumab (targeting HER2)

### Clinical Applications

The methods of the present disclosure may be used in clinical applications to inform various aspects related to cancer in a subject from which the microparticles originated.

The methods of the present disclosure may comprise determining the presence of a cancer in a subject based on the quantification of two or more proteins from a microparticle preparation from a biological sample from the subject. The determination of the presence of the cancer may be done by a trained machine learning algorithm, e.g., a classifier.

In certain embodiments, determining the presence of a cancer in a subject may involve assaying the expression level of a two or more proteins from a microparticle preparation prepared from a biological fluid sample from a subject, to yield a data set comprising respective quantitative measures of each of the two or more proteins, inputting the data set to a trained machine learning algorithm that is configured to generate a classification of said sample as positive or negative for the cancer, and electronically outputting a report that identifies said classification of the sample as positive or negative for the cancer.

Once the presence of a cancer in a subject is determined, that information can be used in various clinically relevant ways.

In some embodiments, the determination of whether or not a subject has a cancer may be used to select the subject to be a candidate for receiving a cancer therapy. As such, in certain embodiments, the methods of the disclosure may comprise determining whether the subject has a cancer and thereby be a candidate for receiving a cancer therapy. Optionally, methods of the disclosure may further comprise treating the selected subject with the cancer therapy.

In some embodiments, the subject may have been previously diagnosed with a cancer and be undergoing ongoing cancer treatment, and the determination of whether the subject has the cancer may be used to monitor the ongoing cancer treatment. In some embodiments, the determination of whether the subject has the cancer may be used to determine whether to continue or change a cancer therapy that the subject has been receiving. In some embodiments, the continued presence of the cancer may indicate that the current cancer therapy requires more time, and thereby be selected to receive an additional administration of the cancer therapy. In some embodiments, the continued presence of the cancer may indicate that the current cancer therapy is inadequate, and the subject may be selected to receive the same cancer therapy at a higher dose, and the subject may optionally be administered the same cancer therapy at the higher dose. In some embodiments, the continued presence of the cancer may indicate that the current cancer therapy is inadequate, and the subject may be selected to receive a different cancer therapy, and the subject may optionally be administered the different cancer therapy.

In certain embodiments, the subject may be in remission, and a determination that the subject has cancer may be determined to be a recurrence of the cancer.

In some embodiments, a cancer therapy may be chemotherapy, hormone therapy, combination therapy, immunotherapy, vaccine therapy, cell-based therapy, radiation therapy, electromagnetic stimulation and/or surgery. In some embodiments, cancer therapy may be administration of a therapeutic agent. The therapeutic agent may be a chemotherapeutic agent, or an immunotherapeutic agent (e.g. a checkpoint inhibitor, a CAR-T, or a cytokine). In certain embodiments, the therapeutic agent may be a small molecule or a biologic, e.g., an antibody, or an engineered cell. In certain embodiments, the therapeutic agent may be a cancer vaccine. Other examples of cancer therapies include radiation therapy (e.g. X-ray, alpha particles emission). Examples of specific therapeutic agents include, for example, cyclophosphamide, chlorambucil, melphalan, methotrexate, cytarabine, fludarabine, 6-mercaptopurine, 5-fluorouracil, vincristine, paclitaxel, vinorelbine, docetaxel, doxorubicin, irinotecan, cisplatin, carboplatin, oxaliplatin, tamoxifen, bicalutamide, anastrozole, exemestane, letrozole, imatinib, gefitinib, erlotinib, rituximab, trastuzumab, gemtuzumab ozogamicin, interferon-alpha, tretinoin, arsenic trioxide, bevacizumab, sorafinib, and sunitinib.

In certain embodiments, a therapeutic agent for cancer therapy may be an immune response modulator. The immune response modulator may be a checkpoint inhibitors, which may include PD-1 inhibitors such as pembrolizumab and nivolumab, and PD-L1 inhibitors such as atezolizumab and durvalumab. In certain embodiments, the immune response modulator may be a cytokine, such as IL-2 and interferon-alpha. Other examples of immune response modulators include CAR-T cell therapies such as tisagenlecleucel and axicabtagene ciloleucel, and monoclonal antibodies such as rituximab (targeting CD20) and trastuzumab (targeting HER2).

It will also be appreciated that an “administration” of a given cancer therapy make take one of various forms depending on the cancer therapy. For example, if the cancer therapy is a surgery, then the administration may be the performance of a surgical procedure. If the cancer therapy is a therapeutic agent, then the administration may be, e.g., oral, subcutaneous, parenteral, intravenous, intracranial, etc. If the cancer therapy is a radiation therapy, then the administration may be a session to receive an emission of a radiation.

### Clinically Relevant Analyses of Biomarkers

The methods of the present disclosure may be used in clinical applications to inform various aspects related to cancer in a subject from which the microparticles originated. In certain embodiments, such clinical application methods may involve comparison of protein expression in microparticles from a test subject with microparticles from one or more control subjects. One of skill in the art would readily recognize appropriate control microparticles from control subjects for various clinical applications.

### Diagnosing Cancer

The present disclosure includes methods of diagnosing cancer in a test subject.

Diagnosing cancer, as described herein, includes, for example, making a determination that a test subject has cancer and making a determination of the specific type of cancer in a test subject based, at least in part, on the results of the analysis of microparticles isolated according to the methods of the present disclosure. Diagnosing cancer may also include the consideration of other signs, symptoms, and test results of the test subject. Symptoms will vary with the type of cancer and may include, for example, weight loss, fatigue, muscle weakness, swollen lymph nodes, chronic cough, blood in stool, recurrent headaches, pain, internal bleeding, partial lung collapse, hoarse voice, shortness of breath, vision problems, loss of appetite, night sweats, fever, confusion, nausea, vomiting, or seizures. Test results may come from imaging studies, such as x-ray, ultrasonography, magnetic resonance imaging (MRI), positron emission technology (PET), or computer tomography (CT). Moreover, diagnosing may include the consideration of factors such as age, sex, family history, previous medical history, or lifestyle, which could indicate an increased likelihood of a diagnosis of cancer.

### Determining Prognosis

In certain aspects, the present disclosure includes methods of determining the prognosis of a test subject with cancer. A prognosis refers to the likely outcome of cancer in a test subject. The prognosis may include, for example, the survival rate, 5-year survival rate, disease-free or recurrence-free survival rate, progression free time period, RECIST criteria, a projection of the course of the illness over time, and/or the likelihood of metastasis of a primary cancer. In addition to the determination of a prognosis based on the expression level of two or more microparticle-associated proteins from a microparticle, the prognosis may also be based on additional factors, such as, for example, Imaging data of cancer recurrence (e.g. MRI, Pet imaging), detection of satellite lesions, changes in tumor size, biopsy assessment, the type, location, and stage of the cancer, the tumor grade, the presence of chromosomal abnormality or abnormal blood cell counts, genomic assessment, physical assessment, clinical chemistries and hematologies, and the age, general health, and predicted response or failure to respond to treatment of the test subject. Further, prognosis may also be based on the results of analysis of one or more characteristics of microparticles in a subject, such as changes in microparticle number, concentration, or microparticle characterization over time.

In certain embodiments, determining the prognosis of the test subject includes comparing the expression level of two or more microparticle-associated proteins from microparticles in the sample from the test subject with the expression level of two or more microparticle-associated proteins in samples from a plurality of control subjects. The plurality of control subjects may include subjects who have cancer and who are known to have a good prognosis or subjects who have cancer and are known to have a bad prognosis. In preferred embodiments, the plurality of control subjects includes both subjects known to have a good prognosis and subjects known to have a bad prognosis. Preferably, the control subjects have the same type of cancer as the test subject. A good prognosis may include, for example, a low likelihood of metastasis, a low likelihood of disease recurrence, a change in pathological status of a cancer to a lower grade of disease involvement, an early stage of cancer, a high likelihood of a positive response to treatment, and a high likelihood of survival or disease-free survival within a time period of greater than 5 years. A bad prognosis may include, for example, a high likelihood of metastasis, a high likelihood of disease recurrence, a high grade of tumor, a late stage of cancer, a low likelihood of a positive response to treatment and a low likelihood of survival or disease-free survival and/or death within a time period of 5 years.

### Determining the Stage of Cancer

In certain aspects, the present disclosure includes methods of determining the stage of cancer in a test subject. Cancer stage describes the extent or severity of the test subject's cancer according to the extent of growth of the primary tumor and the extent of spread in the body. Typically, the stage of cancer is based on the following main factors: location of the primary (original) tumor, tumor size and number of tumors, lymph node involvement (whether or not the cancer has spread to the nearby lymph nodes), and the presence or absence of metastasis.

Solid tumors are classified according to cell type and grade. Different types of cancer stage may be determined by the methods of the present disclosure. These include, for example, clinical staging, pathologic staging, and restaging. Typically, the TNM staging system is used to describe the stage of cancer as determined by the methods of the invention. The TNM Staging System is based on the extent of the tumor (T), the extent of spread to the lymph nodes (N), and the presence of metastasis (M). The T category describes the original (primary) tumor and includes the categories TX (primary tumor cannot be evaluated), TO (no evidence of primary tumor), Tis (carcinoma in situ (early cancer that has not spread to neighboring tissue)), and T1-T4 (size and/or extent of the primary tumor). The N category describes whether or not the cancer has reached nearby lymph nodes and includes the categories NX (regional lymph nodes cannot be evaluated), NO (no regional lymph node involvement (no cancer found in the lymph nodes)), N1-N3 (involvement of regional lymph nodes (number and/or extent of spread)). The M category tells whether there are distant metastases and includes the categories MO (no distant metastasis) and Ml (distant metastasis).

Each cancer type has its own classification system, so letters and numbers do not always mean the same thing for every kind of cancer. Once the T, N, and M are determined, they are combined, and an overall “Stage” of I, II, III, IV is assigned. Sometimes these stages are subdivided as well, using letters such as IIIA and IIIB.

In certain embodiments, determining the stage of cancer in the test subject includes comparing the expression level of two or more microparticle-associated proteins in the sample from the test subject with the expression level of two or more microparticle-associated proteins in samples from a plurality of comparator subjects. The plurality of comparator subjects may include subjects known to have a certain stage of cancer.

Preferably, the plurality of comparator subjects will include at least one comparator subject known to have each of the stages of cancer including Stage 0, Stage I, Stage II, Stage III, and Stage IV.

### Determining Tumor Grade

In certain aspects, the present disclosure includes methods of determining the grade of tumor in a test subject with cancer. Tumor grade is a system used to classify cancer cells in terms of how abnormal they look under a microscope and how quickly the tumor is likely to grow and spread. The methods of the invention allow for a determination of tumor grade based on expression levels of protein biomarkers. Pathologists typically describe tumor grade by four degrees of severity, Grades 1, 2, 3, and 4. The cells of Grade 1 tumors resemble normal cells and tend to grow and multiply slowly. Grade 1 tumors are generally considered to be the least aggressive in behavior. The cells of Grade 3 or Grade 4 tumors do not look like normal cells of the same type. Grade 3 and 4 tumors tend to grow rapidly and spread faster than tumors with a lower grade.

The American Joint Commission on Cancer recommends the following guidelines for grading tumors: GX-grade cannot be assessed (undetermined grade), G1-well-differentiated (low grade), G2-moderately differentiated (intermediate grade), G3-poorly differentiated (high grade), and G4-undifferentiated (high grade). Grading systems are different for each type of cancer. For example, pathologists use the Gleason system to describe the degree of differentiation of prostate cancer cells. The Gleason system uses scores ranging from Grade 2 to Grade 10. Lower Gleason scores describe well-differentiated, less aggressive tumors. Higher scores describe poorly differentiated, more aggressive tumors. Other grading systems include the Bloom-Richardson system for breast cancer and the Fuhrman system for kidney cancer.

In certain embodiments, determining the grade of tumor in the test subject includes comparing the expression level of two or more microparticle-associated proteins from tumor-derived microparticles in the sample from the test subject with the expression level of two or more microparticle-associated proteins in samples from a plurality of comparator subjects. The plurality of comparator subjects may include subjects known to have a tumor of a known grade. Preferably, the plurality of comparator subjects will include at least one comparator subject known to have a tumor of each of the grades including GX, G1, G2, G3, and G4.

### Predicting Response to Treatment

In certain aspects, the present disclosure includes methods of predicting the response of a test subject with cancer to a treatment. Treatments may include, for example, chemotherapy, hormone therapy, combination therapy, immunotherapy, vaccine therapy, cell-based therapy, radiation therapy, electromagnetic stimulation and/or surgery. Examples of specific drug treatments include, for example, cyclophosphamide, chlorambucil, melphalan, methotrexate, cytarabine, fludarabine, 6-mercaptopurine, 5-fluorouracil, vincristine, paclitaxel, vinorelbine, docetaxel, doxorubicin, irinotecan, cisplatin, carboplatin, oxaliplatin, tamoxifen, bicalutamide, anastrozole, exemestane, letrozole, imatinib, gefitinib, erlotinib, rituximab, trastuzumab, gemtuzumab ozogamicin, interferon-alpha, tretinoin, arsenic trioxide, bevacizumab, sorafinib, and sunitinib.

A subject is considered to have a complete response to a treatment if a cancer disappears for any length of time after the treatment. A subject is considered to have a partial response to a treatment if the size of a tumor (usually determined by x-rays) is reduced by more than half, although it remains visible on an x-ray. A subject may present with stable disease in that the cancer is not progressing, changing in features or metastasizing. A subject is considered to not respond to a treatment if the tumor continues to increase in size or new sites of disease appear after the treatment.

In certain embodiments, predicting the response of the test subject to a treatment includes comparing the expression level of two or more microparticle-associated proteins from microparticles in the sample from the test subject with the expression level of two or more microparticle-associated proteins in samples from a plurality of comparator subjects or patients with different types of cancer. The plurality of comparator subjects may include subjects who had cancer and responded to the treatment or subjects who had cancer and did not respond to the treatment. In preferred embodiments, the plurality of comparator subjects includes both subjects who did and did not respond to the treatment. Preferably, the comparator subjects have or had the same type of cancer as the test subject. In certain embodiments, the samples from the comparator subjects were taken from the comparator subjects before administration of the treatment.

### Monitoring Progression of Cancer

In certain aspects, the invention includes methods of monitoring the progression of cancer in a test subject. “Monitoring progression” as used herein may refer to the use of expression levels of protein biomarkers to provide useful information about a test subject or a test subject's health or disease status. The methods of monitoring the progression of cancer as described herein may be used once or multiple times, at irregular or regular intervals, in the treatment and management of cancer in a test subject.

Monitoring progression may include, for example, determination of prognosis, risk-stratification, selection of drug therapy or other treatment, assessment of ongoing drug therapy, determination of effectiveness of treatment, prediction of outcomes, determination of response to therapy, diagnosis of a disease or disease complication, following of progression of a disease or providing any information relating to a test subject's health status over time, selecting test subjects most likely to benefit from experimental therapies with known molecular mechanisms of action, selecting test subjects most likely to benefit from approved drugs with known molecular mechanisms where that mechanism may be important in a small subset of a disease for which the medication may not have a label, screening a population of test subjects to help decide on a more invasive/expensive test, for example, a cascade of tests from a non-invasive blood test to a more invasive option such as biopsy, or testing to assess side effects of drugs used to treat another indication. In certain embodiments, monitoring the progression of cancer can refer to distinguishing between necrotic tissue and cancerous growth after the administration of radiation therapy to a test subject. In particular, monitoring progression may refer to making a determination that cancer in a test subject has progressed from a less advanced to a more advanced stage of cancer between two time points or making a determination that cancer in a test subject has not progressed from a less advanced to a more advanced stage of cancer between two time points.

Monitoring the progression of cancer may include the use of one or more standard clinical techniques such as ultrasound, magnetic resonance imaging, computed tomography scan, single-photon emission computerized tomography, biopsy, or positron emission tomography scan. Results from these tests may be used to supplement or confirm the information gleaned from the expression levels of the microparticle-associated protein biomarkers from microparticles in the test subject for monitoring the progression of cancer.

In certain embodiments, determining the stage monitoring the progression of cancer in the test subject includes comparing the expression level of two or more microparticle-associated proteins from microparticles in the sample from the test subject with the expression level of one or more microparticle-associated proteins in samples from a plurality of comparator subjects. The plurality of comparator subjects may include subjects known to have cancer at different levels of progression. In certain embodiments, the different levels of progression are different stages of cancer, including Stage 0, Stage I, Stage II, Stage III, and Stage IV. In other embodiments, the different levels of progression may be different grades of tumor or different levels of other pathological classifications known in the art.

### Predicting Recurrence of Cancer

In certain aspects, the present disclosure includes methods of predicting or diagnosing the recurrence of cancer in a test subject. “Recurrence of cancer,” as used herein, may refer to a return of cancer in a test subject after treatment and after a period of time during which the cancer cannot be detected. Recurrence of cancer may include a detection of a tumor mass of at least 25% the size of the original tumor by MRI, a return of cancer symptoms, or the appearance of a new tumor of comparable pathology to the original tumor in a different part of the body.

Samples may be taken from the test subject before treatment or at any time after treatment. Typically, the period of time during which the cancer cannot be detected is at least a year and may be a period of several years. The cancer may return to the same place in the body as the original cancer, or it may return to a different place in the body (e.g., metastasis). Cancer may return to the same place in the body as the original cancer even if that part of the body was altered during treatment (e.g., breast cancer may return in the original area or may relocate to other body area such as to the brain). “Local recurrence” means that the cancer has come back at the same place where it first started. “Regional recurrence” means that the cancer has come back in the lymph nodes near the place where it started. “Distant recurrence” means the cancer has come back in another part of the body, some distance from where it started (often the lungs, liver, bone marrow, or brain). The risk of recurrence of cancer in a test subject will depend on the type of cancer, the type of treatment, and the period of time elapsed since the treatment. Predicting the recurrence of cancer typically involves making a determination of the risk of recurrence in the test subject.

### Enumerated Embodiments

The following exemplary embodiments are provided as exemplary.

### Set 1

Embodiment I-1. A method for determining an aspect of a cancer in a subject, the method comprising:
(a) providing a microparticle-enriched fraction from a biological sample from the subject;
(b) quantifying one or more proteins in the fraction, wherein the one or more proteins are selected from one of Tables 2.1, 2.2, 3.1, 4.1, 5.1, 6.1, 7.1, 7.2, 7.3, 8.2-8.4, and 9.2-9.16; and
(c) based on the quantification of the one or more proteins, determining an aspect of the cancer in the subject,
wherein the determining of the aspect of the cancer is selected from:
   i) diagnosing the subject regarding the cancer;
   ii) assessing a risk of the cancer in the subject;
   iii) assessing a risk of recurrence of the cancer in the subject;
   iv) determining presence of the cancer in the subject;
   v) selecting a therapeutic agent to administer to the subject;
   vi) selecting and administering a therapeutic agent to the subject;
   vii) assessing the effectiveness of a previously administered therapeutic agent on the subject;
   viii) assessing the effectiveness of a previously administered therapeutic agent on the subject and continuing administration of the therapeutic agent.

Embodiment 1-2. The method of embodiment I-1, wherein the one or more proteins comprise between 2 and 20 proteins.

Embodiment 1-3. The method of embodiment I-1 or embodiment 1-2, wherein the cancer is a solid tumor.

Embodiment 1-4. The method of any one of embodiments I-1 to 1-3, wherein the solid tumor is a colorectal cancer, a breast cancer, an ovarian cancer, a lung cancer, a brain cancer, a spinal cancer, a pancreatic cancer, a prostate cancer, a renal cancer, a gastric cancer, a sarcoma, or a bladder cancer.

Embodiment 1-5. The method of any one of embodiments I-1 to 1-4, wherein the quantifying of the one or more proteins in the fraction comprises comparing the quantification of the one or more proteins in the fraction against another quantification of the one or more proteins determined in a second biological sample taken from the subject at an earlier or a later time point.

Embodiment 1-6. The method of any one of embodiments I-1 to 1-5, wherein the obtaining of microparticle-enriched fraction comprises centrifugation, ultracentrifugation, affinity purification, filtration, electroporation, affinity binding in solution or solid phase, magnetic beads, immunoprecipitation, microfiltration, or size-exclusion chromatography.

Embodiment I-7. The method of embodiment I-6, wherein the exclusion chromatography comprises a solid phase or and an aqueous liquid phase, wherein the solid phase is an agarose, sepharose, or a combination thereof.

Embodiment I-8. The method of embodiment I-7, wherein the aqueous liquid phase is water.

Embodiment I-9. The method of embodiment I-8, wherein the water is double distilled water.

Embodiment I-10. The method of any one of embodiments I-1 to I-9, wherein the one or more proteins are selected from Table 2.1.

Embodiment I-11. The method of embodiment I-10, wherein the one or more proteins are selected from the group consisting of a Heparin cofactor 2, a Phosphatidylinositol-glycan-specific phospholipase, a Complement C1q, a Biotinidase, a Band 3 anion transport protein, a Hyaluronan-binding protein 2, a Plasma kallikrein, a Kininogen-1, an analog from cDNA FLJ53075, a C4b-binding protein alpha chain, an analog from cDNA FLJ51597, a Cholinesterase, and an Apolipoprotein A.

Embodiment I-12. The method of any one of embodiments I-1 to I-11, wherein at least one of the one or more proteins is a fragment thereof, a variant thereof, a homolog thereof, a congener thereof, a phosphorylated modification thereof or a post-translational modification thereof.

Embodiment I-13. The method of any one of embodiments I-1 to I-12, wherein the biological sample is a biological fluid.

Embodiment 1-14. The method of embodiment 1-13, wherein the biological fluid is or is obtained from: blood or a fraction thereof, lymph, urine, cerebrospinal fluid, ascites, saliva, lavage, semen, glandular fluid, vaginal fluid, exudate, contents of cysts, or feces.

Embodiment 1-15. The method of any one of embodiments I-1 to 1-14, wherein the quantification of the one or more proteins comprises one or a combination of one or more of affinity capture, antibody detection, mass spectroscopy, ELISA, western blot, antibody microarray, or a proximity ligation assay using a selected antibody with nucleic acid tag that can be amplified by primers for detection of small protein quantities.

Embodiment 1-16. The method of embodiment 1-15, wherein the mass spectroscopy is liquid chromatography with tandem mass spectrometry.

Embodiment 1-17. The method of embodiment 1-15, wherein the mass spectroscopy comprises multiple reaction monitoring (MRM), parallel reaction monitoring (PRM) or selected reaction monitoring (SRM).

Embodiment 1-18. The method of any one of embodiments I-1 to 1-14, wherein the quantification of the one or more proteins comprises an assay that utilizes a capture agent where said capture agent is selected from the group consisting of an antibody, an antibody fragment, a nucleic acid-based protein binding reagent, and a small molecule.

Embodiment 1-19. The method of embodiment 1-18, wherein the assay is selected from the group consisting of an enzyme immunoassay (EIA), an enzyme-linked immunosorbent assay (ELISA), and a radioimmunoassay (RIA).

Embodiment 1-20. The method of embodiment 1-19, wherein the quantifying further comprises mass spectrometry (MS) or co-immunoprecipitation-mass spectrometry (co-IP MS).

Embodiment 1-21. The method of any one of embodiments I-1 to 1-9, wherein the one or more proteins comprises a lipid metabolism protein.

Embodiment 1-22. The method of embodiment 1-21, wherein the lipid metabolism protein is PON1.

Embodiment 1-23. The method of any one of embodiments I-1 to 1-9, wherein the one or more proteins comprise a hemostasis protein.

Embodiment 1-24. The method of embodiment 1-23, wherein the hemostasis protein is Factor XI or Platelet Factor 4.

Embodiment 1-25. The method of any one of embodiments I-1 to 1-9, wherein the one or more proteins comprise an extracellular matrix protein.

Embodiment 1-26. The method of embodiment 1-25, wherein the extracellular matrix protein is Tenascin-C or Thrompospondin-1.

Embodiment 1-27. The method of any one of embodiments I-1 to 1-9, wherein the one or more proteins comprise an innate immunity protein.

Embodiment 1-28. The method of embodiment 1-26, wherein the innate immunity protein is Complement Factor H, Complement Component 1 Subcomponent S, or Complement Component 1q.

Embodiment 1-29. A method for determining presence of a cancer-induced host immunosuppressive environment in a subject, the method comprising:
(a) providing a microparticle-enriched fraction from a biological sample from the subject;
(b) quantifying one or more proteins or fragments thereof in the fraction, wherein the one or more one or more proteins include at least one antigen presenting cell (APC) marker or at least one tumor immune suppressor; and
(c) based on the quantification of the one or more proteins, determining the presence of cancer-induced immunosuppression in the subject.

Embodiment 1-30. The method according to embodiment 1-21, wherein the at least one APC marker comprises colony stimulating factor 1 receptor.

Embodiment 1-31. The method according to embodiment 1-21, wherein the at least one tumor immune suppressor comprises Fibrinogen-like protein 1.

Embodiment 1-32. The method according to any one of embodiments 1-29 to 1-31, wherein the biological sample is plasma.

Embodiment 1-33. A method of treating cancer in a subject, the method the method comprising:
(a) providing a microparticle-enriched fraction from a biological sample from the subject;
(b) quantifying one or more proteins or fragments thereof in the fraction, wherein the one or more one or more proteins include at least one antigen presenting cell (APC) marker or at least one tumor immune suppressor; and
(c) determining the presence of cancer-induced immunosuppression in the subject based on the quantification of the one or more proteins; and
(d) administer an effective amount of an immune response modulator to the subject based on the determination of the presence of cancer-induced immunosuppression in the subject, thereby treating the cancer.

Embodiment 1-34. The method according to embodiment 1-33, wherein the at least one APC marker comprises colony stimulating factor 1 receptor.

Embodiment 1-35. The method according to embodiment 1-33, wherein the at least one tumor immune suppressor comprises Fibrinogen-like protein 1.

Embodiment I-36. The method according to any one of embodiments I-33 to I-35, wherein the biological sample is plasma.

Embodiment 1-37. A method of identifying cancer biomarkers in a biological sample from a subject, the method comprising:
receiving quantification data corresponding to a set of microparticle-associated proteins or fragments thereof in the biological sample obtained from at least one cancer cohort comprising a plurality of cancer patients and at least one non-cancer cohort comprising a plurality of non-cancer control subjects;
analyzing the quantification data using a random forest model to generate a first set of candidate biomarkers that are predictive of cancer; and
analyzing the first set of candidate biomarkers with and recursive feature elimination to select one or more subsets of the first set of candidate biomarkers comprising biomarkers that are optimally accurate for multiplex biomarker-based cancer prediction.

### Set II:

Embodiment II-1. A method for analyzing a biological fluid sample of a subject, the method comprising:
(a) providing a microparticle preparation prepared from a biological fluid sample from a subject, wherein the biological fluid sample comprises microparticles;
(b) assaying the expression level of two or more proteins from the microparticle preparation, to yield a data set comprising respective quantitative measures of each of the two or more proteins;
(c) inputting the data set to a trained classifier that is configured to generate a classification of said sample as positive or negative for the cancer at an accuracy of at least 80%; and
(d) electronically outputting a report that identifies said classification of the sample as positive or negative for the cancer.

Embodiment II-2. The method of embodiment II-1, wherein the trained classifier is configured to generate the classification of said sample as positive or negative for the cancer at an accuracy of at least 90%, at least 95%, at least 96%, at least 97%, at least 98%, or at least 99%.

Embodiment II-3. The method of embodiment II-1, wherein the trained classifier was trained with training data obtained from a plurality of training samples, and wherein the training samples are microparticle preparations obtained from biological fluid samples from known cancer patients and known non-cancer subjects.

Embodiment II-4. The method of embodiment II-2, wherein the training data set comprises, for each of the plurality of training samples: (a) a training classification of cancer or non-cancer; and (b) a quantitative measure of at least the two or more proteins.

Embodiment II-5. The method of embodiment II-4, wherein the trained classifier is an algorithm comprising a plurality of coefficients, each of the plurality of the coefficients being associated with one of tie two or more proteins, and wherein the algorithm is configured to generate the classification based on the data set comprising the respective quantitative measures of the two or more proteins and the plurality of coefficients.

Embodiment II-6. The method of embodiment II-1, wherein the two or more proteins comprise between 2 and 20 proteins.

Embodiment II-7. The method of any one of embodiments II-1 to II-6, wherein the cancer is a solid tumor.

Embodiment II-8. The method of embodiment II-7, wherein the solid tumor is a colorectal cancer, a breast cancer, an ovarian cancer, a uterine cancer, a fallopian cancer, a lung cancer, a brain cancer, a spinal cancer, a head or neck cancer, a pancreatic cancer, a prostate cancer, a renal cancer, a gastric cancer, a sarcoma, a liver cancer, an abdominal cancer, a peritoneal carcinoma, or a bladder cancer.

Embodiment II-9. The method of any one of embodiments II-1 to II-8, wherein the providing of the microparticle preparation comprises a use of one or more enrichment processes selected from the group consisting of: centrifugation, ultracentrifugation, density gradients, affinity purification filtration, electroporation, affinity binding in solution or solid phase, magnetic activated sorting, immunoprecipitation, microfiltration, size-exclusion chromatography, and alternating current (AC) electrokinetic separation.

Embodiment II-10. The method of embodiment II-9, wherein the providing of the microparticle preparation comprises use of size-exclusion chromatography, and the microparticles are eluted from a size exclusion chromatography column comprising a solid phase, using water as a mobile phase.

Embodiment II-11. The method of embodiment II-10, wherein the solid phase is an agarose, sepharose, or a combination thereof.

Embodiment II-12. The method of embodiment II-10 or II-11, wherein the water is distilled water.

Embodiment II-13. The method of embodiment II-12, wherein the distilled water is double distilled water.

Embodiment II-14. The method of any one of embodiments II-1 to II-9, wherein the two or more proteins are selected from any one of Tables 2.1, 3.1, 4.1, 5.1, 6.1, 7.1-7.3, 8.2, 9.2, 9.5, 9.8, and 9.11.

Embodiment II-15. The method of embodiment II-14, wherein the two or more proteins are selected from Tables 2.1 or 8.2.

Embodiment II-16. The method of embodiment II-15, wherein the two or more proteins comprise a multiplex of proteins selected from a plurality of multiplexes provided in Table 2.2 or Table 8.3.

Embodiment II-17. The method of embodiment II-16, wherein the multiplex of proteins comprises at least one protein selected from Table 8.4.

Embodiment II-18. The method of embodiment II-17, wherein the multiplex of proteins comprises one or both of CO3 and PROS.

Embodiment II-19. The method of any one of embodiments II-15 to II-18, wherein the cancer is selected from the group consisting of: ovarian cancer, colorectal cancer, lung cancer, and breast cancer.

Embodiment II-20. The method of embodiment II-14, wherein the two or more proteins are selected from Table 9.14.

Embodiment II-21. The method of embodiment II-19, wherein the two or more proteins comprise a multiplex of proteins selected from a plurality of multiplexes provided in Table 9.15.

Embodiment II-22. The method of embodiment II-21, wherein the multiplex of proteins comprises at least one protein selected from Table 9.16.

Embodiment II-23. The method of embodiment II-22, wherein the multiplex of proteins comprises one, two, or three proteins out of HEP2, C4BPB, B3AT, and PHLD.

Embodiment II-24. The method of any one of embodiments 1-20 to II-23, wherein the cancer is selected from the group consisting of ovarian cancer, colorectal cancer, lung cancer, and breast cancer.

Embodiment II-25. The method of embodiment II-14, wherein the cancer is breast cancer, and the two or more proteins are selected from Table 31 or Table 9.11.

Embodiment II-26. The method of embodiment II-25, wherein the two or more proteins comprise a multiplex of proteins selected from a plurality of multiplexes provided in Table 9.12.

Embodiment II-27. The method of embodiment II-26, wherein the multiplex of proteins comprises at least one protein selected from Table 9.13.

Embodiment II-28. The method of embodiment II-27, wherein the multiplex of proteins comprises one, two, or three proteins out of PHLD, FIBA, FIBG, and HEP2.

Embodiment II-29. The method of embodiment II-14, wherein the cancer is lung cancer, and the two or more proteins are selected from Table 5.1 or Table 9.5.

Embodiment II-30. The method of embodiment II-29, wherein the two or more proteins comprise a multiplex of proteins selected from a plurality of multiplexes provided in Table 9.6.

Embodiment II-31. The method of embodiment II-30, wherein the multiplex of proteins comprises at least one protein selected from Table 9.7.

Embodiment II-32. The method of embodiment II-31, wherein the multiplex of proteins comprises one, two, or three proteins out of HEP2, C4PBP, and PROS.

Embodiment II-33. The method of embodiment II-14, wherein the cancer is colorectal cancer, and the two or more proteins are selected from Table 4.1 or Table 9.8.

Embodiment II-34. The method of embodiment II-33, wherein the two or more proteins comprise a multiplex of proteins selected from a plurality of multiplexes provided in Table 9.9.

Embodiment II-35. The method of embodiment II-34, wherein the multiplex of proteins comprises at least one protein selected from Table 9.10.

Embodiment II-36. The method of embodiment II-35, wherein the multiplex of proteins comprises one, two, or three proteins out of C1QB, APOA4, PROS, and ECM1.

Embodiment II-37. The method of embodiment II-14, wherein the cancer is ovarian cancer, and the two or more proteins are selected from any one of Tables 6.1, 7.1, 7.2, 73, and 9.2.

Embodiment II-38. The method of embodiment II-37, wherein the two or more proteins comprise a multiplex of proteins selected from a plurality of multiplexes provided in Table 9.3.

Embodiment II-39. The method of embodiment II-38, wherein the multiplex of proteins comprises at least one protein selected from Table 9.4.

Embodiment II-40. The method of embodiment II-39, wherein the multiplex of proteins comprises one, two, or three proteins out of C4BPB, APOA4, PCGBP, PHLD, HABP2, and FIBA.

Embodiment II-41. The method of any one of embodiments II-1 to II-40, wherein at least one of the two or more proteins is a fragment thereof, a variant thereof, a homolog thereof, a congener thereof, a phosphorylated modification thereof or a post-translational modification thereof.

Embodiment II-42. The method of any one of embodiments II-1 to II-41, wherein the biological fluid is or is obtained from: blood or a fraction thereof, interstitial fluid, synovial fluid, bile, breast milk, lacrimal fluid, menstrual fluid, lymph fluid, urine, cerebrospinal fluid, ascites, saliva, lavage, semen, glandular fluid, vaginal fluid, exudate, contents of cysts, or feces.

Embodiment II-43. The method of embodiment II-42, wherein the fraction of the blood is serum or plasma.

Embodiment II-44. The method of any one of embodiments II-1 to II-42, wherein the two or more proteins are quantified using an affinity capture assay, mass spectroscopy, single-molecule array assay (SIMOA), a proximity extension assay, and protein identification by short epitope mapping, or combinations thereof.

Embodiment II-45. The method of embodiment II-44, wherein the two or more proteins are quantified using the affinity capture assay, and the affinity capture utilizes a capture agent selected from the group consisting of an antibody, an antibody fragment, a nucleic acid-based protein binding reagent, and a small molecule.

Embodiment II-46. The method of any one of embodiments II-1 to II-42, wherein the two or more proteins are quantified using an immunoassay.

Embodiment II-47. The method according to embodiment II-46, wherein the immunoassay is selected from the group consisting of: enzyme-linked immunosorbent assay (ELISA), enzyme immunoassay (EIA), radioimmunoassay (RIA), antibody detection, immunohistochemistry, western blot, antibody microarray assay, and a proximity ligation assay using a selected antibody with nucleic acid tag that can be amplified by primers for detection of small protein quantities, or a combination thereof.

Embodiment II-48. The method of embodiment II-47, wherein the immunoassay is selected from the group consisting of ELISA, EIA, and RIA.

Embodiment II-49. The method of embodiment II-14, wherein the two or more proteins comprises a lipid metabolism protein.

Embodiment II-50. The method of embodiment II-49, wherein the lipid metabolism protein is PON1.

Embodiment II-51. The method of embodiment II-14, wherein the one or more proteins comprise a hemostasis protein.

Embodiment II-52. The method of embodiment II-51, wherein the hemostasis protein is Factor XI or Platelet Factor 4.

Embodiment II-53. The method of embodiment II-14, wherein the one or more proteins comprise an extracellular matrix protein.

Embodiment II-54. The method of embodiment II-53, wherein the extracellular matrix protein is Tenascin-C or Thrombospondin-1.

Embodiment II-55. The method of embodiment II-14, wherein the one or more proteins comprise an innate immunity protein.

Embodiment II-56. The method of embodiment II-55, wherein the innate immunity protein is, or is a subunit of. Complement Factor H, Complement Component 1 Subcomponent S, or Complement Component 1q.

Embodiment II-57. The method of any one of embodiments II-1 to II-56, wherein the subject previously was diagnosed as having a cancer that went into remission.

Embodiment II-58. The method of any one of embodiments II-1 to II-57, the method further comprising: (e) determining whether the subject is a candidate for receiving a cancer therapy based on the classification.

Embodiment II-59. The method of embodiment II-58, wherein the subject is the candidate, and the method further comprises treating the subject with the cancer therapy.

Embodiment II-60. A method of monitoring cancer treatment in a subject, the method comprising:
a. assessing a biological fluid sample from a subject that previously was receiving a cancer therapy, in accordance with any one of embodiments II-1 to II-56 to receive a classification of said sample as positive or negative for the cancer; and
b. selecting the subject to be a candidate to receive at least one additional administration of the cancer therapy based on the classification.

Embodiment II-61. The method according to embodiment II-60, further comprising administering the at least one additional administration of the cancer therapy to the subject.

Embodiment II-62. The method of embodiment II-60 or II-61, wherein the at least one additional administration is characterized by an increased dose of the cancer therapy.

Embodiment II-63. A method of monitoring cancer treatment in a subject, the method comprising:
a. assessing a biological fluid sample from a subject that previously was administered a therapeutic agent for treating a cancer, in accordance with any one of embodiments II-1 to II-56 to receive a classification of said sample as positive or negative for the cancer; and
b. selecting the subject to be a candidate to receive at least one dose of a different therapeutic agent based on the classification.

Embodiment II-64. The method according to embodiment II-63, further comprising administering the different therapeutic agent to the subject in an amount effective to treat the cancer.

Embodiment II-65. A method for determining presence of a cancer in a subject, the method comprising:
(a) providing a microparticle preparation prepared from a biological fluid sample from a subject, wherein the biological fluid sample comprises microparticles;
(b) quantifying two or more proteins in the fraction; and
(c) based on the quantification of the two or more proteins, determining the presence of the cancer in the subject,
wherein the two or more proteins are selected from any one of Tables 2.1, 3.1, 4.1, 5.1, 6.1, 7.1-7.3, 8.2, 9.2, 9.5, 9.8, 9.11.

Embodiment II-66. The method of embodiment II-65, wherein the two or more proteins comprise between 2 and 20 proteins.

Embodiment II-67. The method of embodiment II-65 or II-66, wherein the cancer is a solid tumor.

Embodiment II-68. The method of embodiment II-67, wherein the solid tumor is a colorectal cancer, a breast cancer, an ovarian cancer, a uterine cancer, a fallopian cancer, a lung cancer, a brain cancer, a spinal cancer, a head or neck cancer, a pancreatic cancer, a, prostate cancer, a renal cancer, a gastric cancer, a sarcoma, a liver cancer, an abdominal cancer, a peritoneal carcinoma, or a bladder cancer.

Embodiment II-69. The method of any one of embodiments II-65 to II-68, wherein the two or more proteins are selected from Tables 2.1 or 8.2.

Embodiment II-70. The method of embodiment II-69, wherein the two or more proteins comprise a multiplex of proteins selected from a plurality of multiplexes provided in Table 2.2 or Table 8.3.

Embodiment II-71. The method of embodiment II-70, wherein the multiplex of proteins comprises at least one protein selected from Table 8.4.

Embodiment II-72. The method of embodiment II-71, wherein the multiplex of proteins comprises one or both of (03 and PROS.

Embodiment II-73. The method of any one of embodiments II-66 to II-72, wherein the cancer is selected from the group consisting of ovarian cancer, colorectal cancer, lung cancer, and breast cancer.

Embodiment II-74. The method of any one of embodiments II-65 to II-68, wherein the two or more proteins are selected from Table 9.14.

Embodiment II-75. The method of embodiment II-74, wherein the two or more proteins comprise a multiplex of proteins selected from a plurality of multiplexes provided in Table 9.15.

Embodiment II-76. The method of embodiment II-75, wherein the multiplex of proteins comprises at least one protein selected from Table 9.16.

Embodiment II-77. The method of embodiment II-76, wherein the multiplex of proteins comprises one, two, or three proteins out of HEP2, C4BPB, B3AT, and PHLD.

Embodiment II-78. The method of any one of embodiments II-74 to II-77, wherein the cancer is selected from the group consisting of ovarian cancer, colorectal cancer, lung cancer, and breast cancer.

Embodiment II-79. The method of any one of embodiments II-65 to II-68, wherein the cancer is breast cancer, and the two or more proteins are selected from Table 3.1 or Table 9.11.

Embodiment II-80. The method of embodiment II-79, wherein the two or more proteins comprise a multiplex of proteins selected from a plurality of multiplexes provided in Table 9.12.

Embodiment II-81. The method of embodiment II-0, wherein the multiplex of proteins comprises at least one protein selected from Table 9.13.

Embodiment II-82. The method of embodiment II-81, wherein the multiplex of proteins comprises one, two, or three proteins out of PHLD, FIBA, FIBG, and HEP2.

Embodiment II-83. The method of any one of embodiments II-65 to II-68, wherein the cancer is lung cancer, and the two or more proteins are selected from Table 5.1 or Table 9.5.

Embodiment II-84. The method of embodiment II-83, wherein the two or more proteins comprise a multiplex of proteins selected from a plurality of multiplexes provided in Table 9.6.

Embodiment II-85. The method of embodiment II-84, wherein the multiplex of proteins comprises at least one protein selected from Table 9.7.

Embodiment II-86. The method of embodiment II-85, wherein the multiplex of proteins comprises one, two, or three proteins out of HEP2, C4PBP, and PROS.

Embodiment II-87. The method of any one of embodiments II-65 to II-68, wherein the cancer is colorectal cancer, and the two or more proteins are selected from Table 4.1 or Table 9.8.

Embodiment II-88. The method of embodiment II-87, wherein the two or more proteins comprise a multiplex of proteins selected from a plurality of multiplexes provided in Table 9.9.

Embodiment II-89. The method of embodiment II-88, wherein the multiplex of proteins comprises at least one protein selected from Table 9.10.

Embodiment II-90. The method of embodiment II-89, wherein the multiplex of proteins comprises one, two, or three proteins out of C1QB, APOA4, PROS, and ECM1.

Embodiment II-91 The method of any one of embodiments II-65 to II-68, wherein the cancer is ovarian cancer, and the two or more proteins are selected from any one of Tables 6.1, 7.1, 7.2, 7.3, and 9.2.

Embodiment II-92. The method of embodiment II-91, wherein the two or more proteins comprise a multiplex of proteins selected from a plurality of multiplexes provided in Table 9.3

Embodiment II-93. The method of embodiment II-92, wherein the multiplex of proteins comprises at least one protein selected from Table 9.4.

Embodiment II-94. The method of embodiment II-93, wherein the multiplex of proteins comprises one, two, or three proteins out of C4BPB, APOA4, PCGBP, PHLD, HABP2, and FIBA.

Embodiment II-95. The method of any one of embodiments II-65 to II-94, wherein the two or more proteins comprise a lipid metabolism protein, an extracellular matrix protein, or an innate immunity protein.

Embodiment II-96. The method of embodiment II-95, wherein the two or more proteins comprise the lipid metabolism protein, and the lipid metabolism protein is PON.

Embodiment II-97. The method of embodiment II-95, wherein the two or more proteins comprise the hemostasis protein, and the hemostasis protein is Factor XI or Platelet Factor 4.

Embodiment II-98. The method of embodiment II-95, wherein the two or more proteins comprise the extracellular matrix protein, and the extracellular matrix protein is Tenascin-C or Thrombospondin-1.

Embodiment II-99. The method of embodiment II-95, wherein the two or more proteins comprise the innate immunity protein, and the innate immunity protein is, or is a subunit of: Complement Factor H, Complement Component 1 Subcomponent S, or Complement Component 1q.

Embodiment II-100. The method of any one of embodiments II-65 to II-99, wherein at least one of the two or more proteins is a fragment thereof, a variant thereof, a homolog thereof, a congener thereof, a phosphorylated modification thereof or a post-translational modification thereof.

Embodiment II-101. The method of any one of embodiments II-65 to II-100, wherein the biological fluid is or is obtained from: blood or a fraction thereof, interstitial fluid, synovial fluid, bile, breast milk, lacrimal fluid, menstrual fluid, lymph fluid, urine, cerebrospinal fluid, ascites, saliva, lavage, semen, glandular fluid, vaginal fluid, exudate, contents of cysts, or feces.

Embodiment II-102. The method of embodiment II-101, wherein the fraction of the blood is serum or plasma.

Embodiment II-103. The method of any one of embodiments II-65 to II-101, wherein the providing of the microparticle-preparation comprises use of size-exclusion chromatography, and the microparticles are eluted from a size exclusion chromatography column comprising a solid phase, using water as a mobile phase.

Embodiment II-104. The method of embodiment II-103, wherein the water is distilled water.

Embodiment II-105. The method of any one of embodiments II-65 to II-104, wherein the two or more proteins are quantified using an immunoassay.

Embodiment II-106. The method of embodiment II-105, wherein the immunoassay is selected from the group consisting of ELISA, EIA, and RIA.

Embodiment II-107. The method of any one of embodiments II-65 to II-106, the method further comprising: (e) determining whether the subject is a candidate for receiving a cancer therapy based on the classification.

Embodiment II-108. The method of embodiment II-107, wherein the subject is the candidate, and the method further comprises treating the subject with the cancer therapy.

Embodiment II-109. A method for determining presence of a cancer-induced host immunomodulated environment in a subject, the method comprising:
(a) providing a microparticle preparation from a biological fluid sample from the subject;
(b) quantifying two or more proteins in the microparticle preparation, wherein the two or more proteins include at least one antigen presenting cell (APC) marker or at least one tumor immune suppressor; and
(c) based on the quantification of the two or more proteins, determining the presence of the cancer-induced immunomodulation in the subject.

Embodiment II-110. A method of treating cancer in a subject, the method the method comprising:
(a) providing a microparticle preparation from a biological sample from the subject;
(b) quantifying two or more proteins in the microparticle preparation, wherein the two or more proteins include at least one antigen presenting cell (APC) marker or at least one tumor immune suppressor;
(c) determining presence of cancer-induced immunomodulation in the subject based on the quantification of the two or more proteins; and
(d) administering an effective amount of an immune response modulator to the subject based on the determination of the presence of cancer-induced immunomodulation in the subject, thereby treating the cancer.

Embodiment II-111. The method according to embodiment II-109 or II-110, wherein the at least one APC marker comprises colony stimulating factor 1 receptor.

Embodiment II-112. The method according to embodiment II-109 or II-110, wherein the at least one tumor immune suppressor comprises Fibrinogen-like protein 1.

Embodiment II-113. The method of any one of embodiments II-109 to II-112, wherein the cancer-induced immunomodulation is a cancer-induced immunosuppression.

Embodiment II-114. The method of any one of embodiments II-109 to II-112, wherein the cancer is a solid tumor.

Embodiment II-115. The method of embodiment II-113, wherein the solid tumor is a colorectal cancer, a breast cancer, an ovarian cancer, a uterine cancer, a fallopian cancer, a lung cancer, a brain cancer, a spinal cancer, a head or neck cancer, a pancreatic cancer, a prostate cancer, a renal cancer, a gastric cancer, a sarcoma, a liver cancer, an abdominal cancer, a peritoneal carcinoma, or a bladder cancer.

Embodiment II-116. The method of any one of embodiments II-109 to II-115, wherein the biological fluid is or is obtained from: blood or a fraction thereof, interstitial fluid, synovial fluid, bile, breast milk, lacrimal fluid, menstrual fluid, lymph fluid, urine, cerebrospinal fluid, ascites, saliva, lavage, semen, glandular fluid, vaginal fluid, exudate, contents of cysts, or feces.

Embodiment II-117. The method of embodiment II-116, wherein the fraction of the blood is serum or plasma.

Embodiment II-118. The method of any one of embodiments II-109 to II-117, wherein the providing of the microparticle-preparation comprises use of size-exclusion chromatography, and the microparticles are eluted from a size exclusion chromatography column comprising a solid phase, using water as a mobile phase.

Embodiment II-119. The method of embodiment II-118, wherein the water is distilled water.

Embodiment II-120. The method of any one of embodiments II-109 to II-119, wherein the two or more proteins are quantified using an immunoassay.

Embodiment II-121. The method of embodiment II-120, wherein the immunoassay is selected from the group consisting of ELISA, EIA, and RIA.

Embodiment II-122. A method comprising:
a) providing a plurality of microparticle preparations, each of the plurality of microparticle preparations being prepared from a plasma or serum sample from one of a plurality of subjects, the plurality of subjects comprising cancer patients and non-cancer subjects;
b) using mass spectrometry, determining quantitative measures of a plurality of proteins in each of the plurality of microparticle preparations, wherein the plurality of proteins are selected from: the proteins of any one of Tables 21, 2.2, 3.1, 4.1, 5.1, 6.1, 7.1, 72, 7.3, 8.2-8.4, and 9.2-9.16.
c) preparing a training data set indicating, for each sample, values indicating:
   (i) classification of cancer class or non-cancer class; and
   (ii) quantitative measures, respectively, of the plurality of proteins; and
d) training a classifier on the training data set, wherein training generates one or more classification rules that classify a new sample as belonging to the cancer class or the non-cancer class.

Embodiment II-123. A computer system comprising:
(a) a processor; and
(b) a memory, coupled to the processor, the memory storing a module comprising:
   (i) test data for a sample from a subject, the test data including values indicating a quantitative measure of two or more proteins in a microparticle preparation from a biological fluid sample, wherein the two or more proteins are selected from the proteins of any one of Tables 2.1, 2.2, 3.1, 4.1, 5.1, 6.1, 7.1, 7.2, 7.3, 8.2-8.4, and 9.2-9.16;
   (ii) a trained classifier configured to, based on the test data, classify the subject as having a cancer or not having the cancer; and
   (iii) computer executable instructions for implementing the classifier on the test data.

Embodiment II-124. The computer system of embodiment II-122, wherein the classifier is configured to have an accuracy of at least 80%, at least 90%, at least 95%, at least 96%, at least 97%, at least 98%, or at least 99%.

### EXAMPLES

### Example 1: Mass Spectroscopy-Based Quantification of MAPs in Cohort 1

### Summary of Example

In order to assess the potential utility of plasma derived microparticle-associated proteins as biomarkers for cancer, proteomics analysis was performed on a group of 25 non-cancer control subject plasma and 94 plasma samples from lung, colorectal, breast and ovarian cancer patients. After LC-MS/MS, 1441 proteins were identified as being expressed in at least one sample. After differential expression analysis comparing plasma from cancer patients and plasma from non-cancer control subjects, 133 proteins that were more than 2-fold up or down regulated were identified—40 up-regulated and 93 down regulated. ROC analysis was also performed on a set of 853 proteins seen expressed in a majority of samples. This analysis identified 60 different proteins which had ROC area under the sensitivity/specificity curves that were greater than 0.75, with the top 10 proteins identified having AUCs ranging from 0.839 to 0.921. When a group of five of the top 20 proteins having divergent biological functions were pooled, the AUC of this 5-plex demonstrated an AUC of 0.973. Taken together, this initial analysis of microparticle associated proteins demonstrates a significant cohort of proteins that are differentially expressed between cancer patients and non-cancer control subjects, as well as sub-sets of these proteins that demonstrate very high utility as potential diagnostic biomarker panels for the presence of cancer. These biomarkers, many of which have not been previously identified as cancer biomarkers, may be useful for, e.g., accurately identifying patients at risk of cancer recurrence as well as detection of cancer and/or predictors of response to therapy.

### Subjects Used in Study

119 patient plasma samples were acquired as follows: plasma samples from 25 non-cancer control subjects, plasma samples from 25 stage 3/4 ovarian cancer patients, 25 stage 2/3 breast cancer plasma, plasma samples from 25 stage 3/4 colorectal patients, and plasma from 19 stage 3/4 non small-cell lung cancer patients. All of the 119 subjects in this study may be referred to herein in an aggregate as “Cohort 1”.

### Sample Collection

Plasma samples from human subjects (cancer patients and non-cancer controls) were obtained with medical consent and provided to the labs with medical annotation and stored at (−80° C.) from a commercial biorepository Samples were secured with the collaboration of a commercial vendor (Proteogenix (USA)) following informed consent.

Inclusion criteria required samples to be derived from either non-cancer control subjects or patients with histopathologically defined cancers. The samples were collected via venipuncture in EDTA tubes and centrifuged for 10 minutes at 1,500 xg to remove large debris. The plasma was de-identified, transferred into clean 1.5 mL Eppendorf tubes and stored −80° C. All de-identified plasma samples were transferred on dry ice to the Applicant's laboratory (Durham, NC) and stored at −80° C. until time of study.

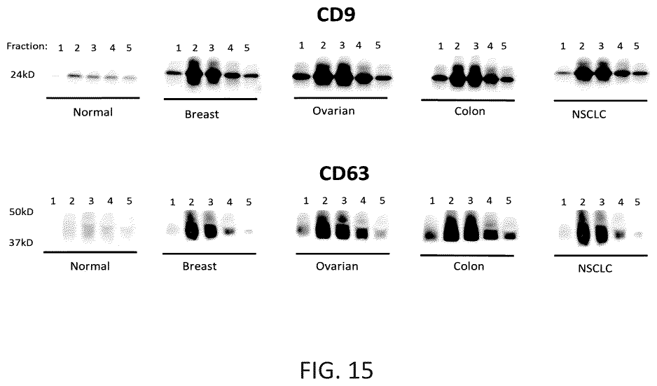

All samples were received in a frozen state. All serum samples (n=120) were processed contemporaneously. Plasma samples (1 mL) were thawed on ice to room temperature, and 500 μL of plasma samples loaded onto prewashed and equilibrated agarose SEC columns (bed volume 5 mL; Izon®) and eluted isocratically with double distilled water at low rate of gravity feed. Once plasma has entered the loading frit, 2.5 mL Buffer was loaded. Once the column stopped flowing, an additional 400 μL of Buffer was loaded onto the column and effluent collected (Fraction 1) and subsequently repeated until the flow stopped and repeated for Fractions 2-5. Fractions yielded two partially resolved peaks when monitored for particle and protein content as well as presence of canonical proteins associated with the high molecular weight microvesicles. Fractions 1-5 were collected and denoted “microparticle-enriched fractions” or “microparticle preparations”. Western Blots were run on fractions 1-5 recovered and probed for the tetraspanin proteins CD9 and CD63. Tetraspanins are a family of membrane proteins found in all multicellular eukaryotes. As such, an increased concentration of CD9 and CD63 in the fractions provide a positive control confirming the enrichment of microparticles (which as noted above typically comprise cell membrane material). Examples of CD9 and CD63 Western Blots of the fractions 1-5 are shown in FIG. 15. As shown in the figure, fraction 2, followed by fraction 3, are the most enriched in microparticles. A pool of equivalent amounts of microparticle-enriched fractions 2-5 or 1-5 were then subjected to proteomic analysis using Liquid Chromatography with tandem mass spectrometry (LC-MS/MS) as described below.

### Protein Extraction and Digestion for Mass Spectrometry

After extracting the protein from the microparticle samples, the protein was desalted on spin columns, and were subjected to trypsin digestion; followed by reduction and alkylation. Following digestion, the solution was centrifuged at 12,000×g at room temperature for 20 min to collect the digested peptides. The filtrates were collected and lyophilized to obtain the dry powder. The peptide samples were dissolved in buffer and mixed with anhydrous acetonitrile and vortexed and the samples were ready for MS. The peptides were analyzed using LC-MS/MS methods.

The peptide samples were vortexed and 60 μl was combined with an equal volume of lysis buffer resuspended (10% SDS, 100 mM TEAB pH 8.5), vortexed and super-sonicated, and heated to 90° C. for 5 minutes. Then the samples were centrifuged at 14,000 rpm and the supernatant were collected for BCA assay and S-Trap (S-Trap™ micro MS sample prep kit, Protifi) procedures as described by the manufacturers. Briefly, 50 μg of each sample was normalized with 5% SDS 100 mM TEAB, then reduced with 10 mM final concentration of DTT at 55° C. for 15 minutes and alkylated with 30 mM final concentration of IAM at room temperature for 10 minutes. The protein samples were then acidified with 27.5% phosphoric acid to reach pH≤1. Proteins were trapped into the S-Trap column by centrifuge at 10,000 g for 30 seconds and washed with 100 mM TEAB (final) in 90% methanol repeatedly. Trypsin/LysC (Cat No.: A40007, Thermo Fisher Scientific) was added to protein samples at 1:10 w/w ratio for overnight digestion at 37° C. Digested samples were quenched with 0.2% formic acid. Samples were then eluted from the S-Trap column with sequential addition and centrifuge of buffer 1 (50 mM TEAB), buffer 2 (0.2% formic acid) and buffer 3 (50% acetonitrile). The eluted solution was pooled and subsequently dried by SpeedVac. (Savant™ SpeedVac™ SPD120, Thermo Fisher Scientific).

Peptide Fractionation: Fifty percent of each eluted samples were dried by speed-vac and reconstituted in 50 μl of MS injection buffer. The remaining 50% of peptides aliquot of each sample was taken and pooled together as library composite. The library composite samples were fractionated into 96 fractions with a high pH reverse phase offline HPLC fractionator (Vanquish™, Thermo Fisher Scientific). Mobile phase A is DI H₂O with 5.3 mM Formic Acid, 17.3 mM Ammonium Hydroxide, pH 9.3; mobile phase B is Acetonitrile (Optima™, LC/MS grade, Fisher Chemical™) with 5.3 mM Formic Acid, 17.3 mM Ammonium Hydroxide, pH 9.3. Gradient of separation is displayed in Table 1.1. Total 96 fractions were then combined into 12 fractions and ready for LC-MS/MS analysis.

TABLE 1.1
High pH Reverse Phase HPLC Fractionation Gradient Information

| Time[min] | Flow[ml/min] | % B |
|---|---|---|
| 0.00 | 0.500 | 2.0 |
| 1.00 | 0.500 | 6.0 |
| 12.00 | 0.500 | 20.0 |
| 30.00 | 0.500 | 28.0 |
| 50.00 | 0.500 | 65.0 |
| 53.00 | 0.500 | 98.0 |
| 57.00 | 0.500 | 98.0 |
| 59.00 | 0.500 | 2.0 |
| 60.00 | 0.500 | 2.0 |

### LC-MS/MS Analysis

All fractionated samples were analyzed by nano flow HPLC (Ultimate 3000, Thermo Fisher Scientific) followed by Orbitrap Eclipse™ Tribrid™ (Thermo Fisher Scientific). Nanospray Flex™ Ion Source (Thermo Fisher Scientific) was equipped with Column Oven (PRSO-V2, Sonation) to heat up the nano column (Aurora Ultimate, 250 mm×75 μm ID, 1.7 μm C18, IonOpticks) for peptide separation. The nano LC method is water acetonitrile based 120 minutes long with 0.3 μL/min flowrate. For each library fractions, all peptides were first engaged on a trap column (Cat. No: 164535, Thermo Fisher Scientific) and then were delivered to the separation nano column by the mobile phase. A specific of gradient information was indicated in Table 1.2.

For DDA library construction, a DDA library specific DDA MS2-based mass spectrometry method on Eclipse™ was used to sequence fractionated peptides that were eluted from the nano column. The ionized peptides were fractionated by FAIMS Pro™ using a 3-CV (−50, -65, -85 V) method. For the full MS, 120,000 resolution was used with the scan range of 350 m/z-1500 m/z. For the dd-MS(MS2), 30,000 resolution was used, and Isolation window is 1.6 Da. ‘Standard’ AGC target and ‘Auto’ Max Ion Injection Time (Max IT) were selected for both MS1 and MS2 acquisition. Collision Energy mode was ‘Fixed’ and total cycle time is 1 sec.

For DIA analytical samples, a high-resolution full MS scan followed by two segment DIA methods was used for the DIA data acquisition. For the full MS scan, 120,000 resolution was used for the range of 400 m/z-1200 m/z with ‘Standard’ AGC target, 50 ms Max IT and −55 V FAIMS CV. For both DIA segments, details of isolation windows (IW) and precursor mass range are shown in Table 1.3 & Table 1.4. For DIA fragments scan, 30,000 resolution was used for the range of 110 m/z-1,800 m/z with ‘Standard’ AGC target and ‘Auto’ Max IT.

TABLE 1.2
nano LC-MS/MS Gradient Information.

| Time[min] | Flow[μl/min] | % B |
|---|---|---|
| 0.00 | 0.300 | 2.0 |
| 2.10 | 0.300 | 2.0 |
| 3.00 | 0.300 | 4.0 |
| 98.00 | 0.300 | 35.0 |
| 108.00 | 0.300 | 65.0 |
| 109.00 | 0.300 | 100.0 |
| 114.00 | 0.300 | 100.0 |
| 115.00 | 0.300 | 2.0 |
| 120.00 | 0.300 | 2.0 |

TABLE 1.3
DIA segment 1 Precursor Scan Range Information.
Segment 1
(400-800 m/z, IW 15 m/z, Overlap 1 m/z)

| | |
|---|---|
| 399.5-415.5 | 609.5-625.5 |
| 414.5-430.5 | 624.5-640.5 |
| 429.5-445.5 | 639.5-655.5 |
| 444.5-460.5 | 654.5-670.5 |
| 459.5-475.5 | 669.5-685.5 |
| 474.5-490.5 | 684.5-700.5 |
| 489.5-505.5 | 699.5-715.5 |
| 504.5-520.5 | 714.5-730.5 |
| 519.5-535.5 | 729.5-745.5 |
| 534.5-550.5 | 744.5-760.5 |
| 549.5-565.5 | 759.5-775.5 |
| 564.5-580.5 | 774.5-790.5 |
| 579.5-595.5 | 789.5-800.5 |
| 594.5-610.5 | |

TABLE 1.4
DIA segment 2 Precursor Scan Range Information
Segment 2
(800-1200 m/z, IW 25 m/z, Overlap 1 m/z)

| | |
|---|---|
| 799.5-825.5 | 999.5-1025.5 |
| 824.5-850.5 | 1024.5-1050.5 |
| 849.5-875.5 | 1049.5-1075.5 |
| 874.5-900.5 | 1074.5-1100.5 |
| 899.5-925.5 | 1099.5-1125.5 |
| 924.5-950.5 | 1024.5-1150.5 |
| 949.5-975.5 | 1049.5-1175.5 |
| 974.5-1000.5 | 1074.5-1200.5 |

### Example 2: Bioinformatic Analysis of Quantified MAPs

An in-house developed software tool was used for DDA spectral library construction and subsequent DIA analysis. The analysis used raw data provided as described in Example 1 as input files and set corresponding parameters based on human database, then performed identification and quantitative analysis. The identified peptides satisfied FDR <=1% will be used to construct the final spectral library. GO, COG, Pathway functional annotation analysis were also performed in above pipeline. MSstats, which core algorithm is linear mixed effect model, processed DIA quantification result data according to the predefined comparison group, and then performed the significance test based on the model. Thereafter, differential protein screening was performed, and Fold Change >2 and P-Value<0.05 was defined as significant difference. Based on the quantitative comparison results, the differential proteins between comparison groups were found, and finally function enrichment analysis, protein-protein interaction (PPI) and subcellular localization analysis of the differential proteins were performed.

In this project, Eclipse was used to acquire mass spectrometry (MS) data for 119 samples in Data Independent Acquisition (DIA) mode, 9348 peptide and 1447 protein were quantitated. Quantification of peptides and proteins was performed. In this project, MSstats software package was applied to intra-system error correction, normalization for each sample. Then based on the predefined comparison groups and the linear mixed effect model, the significance of differentially expressed proteins (DEPs) was evaluated. Two filtration criteria (Fold change (increase or decrease)>2 and p-value<0.05) were used to get significant differential proteins that were then analyze by volcano plots and receiver operating characteristic (ROC) curves for statistical comparison to control or other cancer cohorts

After DIA analysis of the samples, 1408 unique proteins were identified across all 119 samples.

Differential expression analysis of this data set was initially stratified by comparing all cancers together as a group (pan cancer) constituting 94 cancer samples, and comparing expression of each protein to the 25 non-cancer control samples as the second group. Differential expression of each was analyzed based on Log2 Fold Change (Log2FC) between the cancer group and non-cancer control group where a positive Log2FC value represents a protein seen more abundantly in the cancer group and a negative Log2FC represents a protein seen less abundantly in the cancer group vs the non-cancer control group. All biomarkers in Table 5 have a p-value of less than 0.05. A p-value was determined for the cancer/non-cancer comparison for each protein.

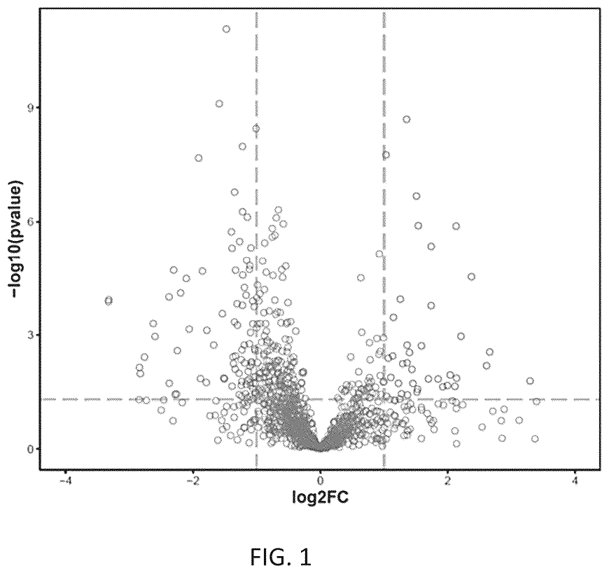

The 1408 unique proteins quantified in the LC-MS/MS study were plotted in FIG. 1 (volcano plot), which is a visual representation of the distribution of all proteins across the entire data set. The X-axis represents fold change of protein and the Y-axis represents the p-value of protein. Thresholds of Log2FC greater or less than 1 and P-value<0.05 were set to identify those proteins most likely to have statistically significantly differential expression. The data was further stratified using adjusted p-value<0.05 to minimize the false discovery rate of hits. In addition to analysis using volcano plot, hierarchical clustering of differentially expressed proteins was performed in order to assess relationships between differentially expressed proteins, as well as identifying those proteins that are differentially expressed across all 4 cancer types, as opposed to any single cancer type.

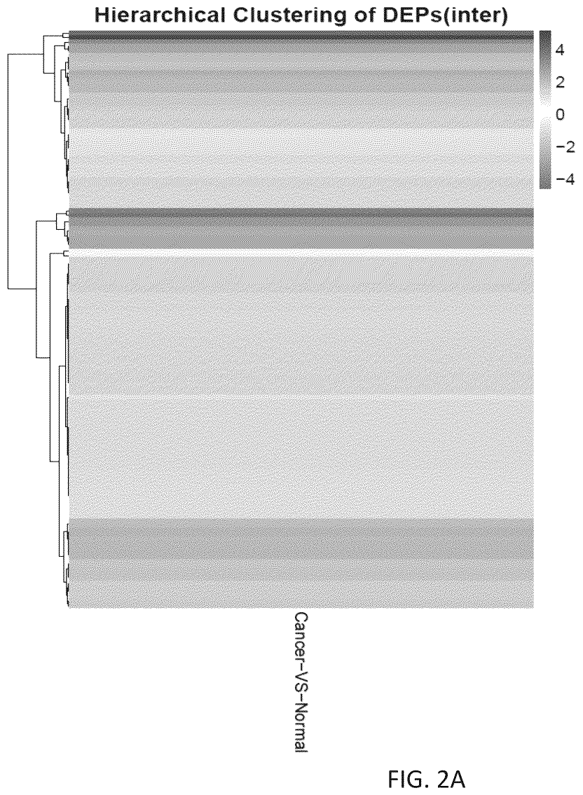

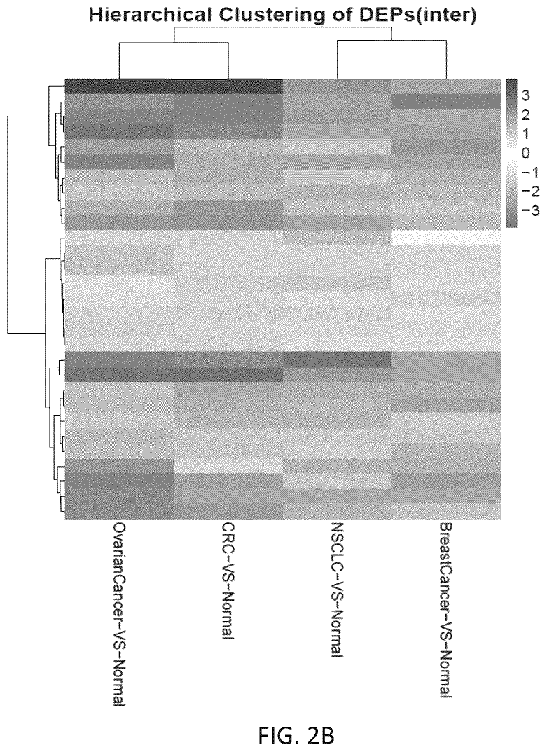

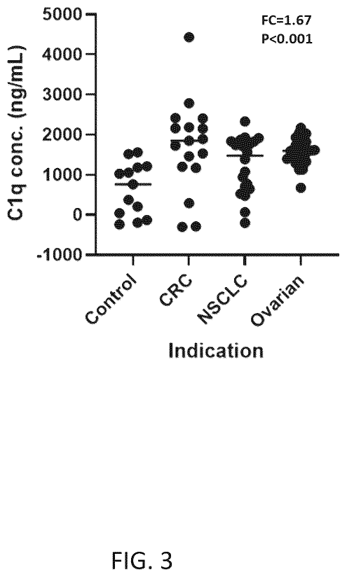

FIG. 2A represents a heat map of differentially expressed microvesicle-associated proteins in microparticle preparations prepared from across all 4 cancer types vs. microparticle preparations prepared from non-cancer control subjects, where red represents significantly upregulated proteins and blue represents significantly down regulated proteins. FIG. 2B represents the same data set, broken out into the 4 included cancer types, showing that the population of proteins identified in FIG. 2A are each seen differentially expressed in each of the 4 cancer subtypes, strengthening the interpretation that this is indeed a pan cancer fingerprint and not one that represents over fitting of the data based on a single one of the cancer subtypes. Based upon these data, specific microparticle associated proteins individually and in groups were identified to perform additional confirmatory studies. One such protein complement C1q was subjected to immune analysis in order to confirm that the differential expression as determined by LC/MS was also reflected in immune analysis, as described below and shown in FIG. 3.

### Receiver Operating Characteristic (ROC) Curve Analysis of Biomarkers

Having seen differential expression in these patient sample cohorts, an assessment was made of the potential of one or more of these biomarkers to accurately identify cancer patient plasma vs non-cancer patient plasma within the initial study cohort (as presented above, the study cohort consisted of plasma from 25 non-cancer control subjects, 25 stage 3/4 ovarian cancer patients, 25 stage 2/3 breast cancer patients, 25 stage 3/4 colorectal patients and 19 stage 3/4 non small-cell lung cancer patients. Predictive biomarkers can be determined by plotting a receiver operating characteristic (ROC) curve which plots the predicted true positive vs false positive rate of an analyte across the detection range of the analyte. In the case of the present study, an ROC curve was plotted for each biomarker across its detection range in the microparticle preparations from the cancer and non-cancer cohorts. In an ideal situation, a quantitative cutoff can exist that will perfectly distinguish cancer from non-cancer samples. In this ideal situation, the area under the curve (AUC) of the ROC curve is 1. By contrast, a random analyte which has no predictive value has an AUC of 0.5. As such, a biomarker having an AUC of the ROC curve that is closer to 1 would be considered to have a higher predictive value for distinguishing cancer from non-cancer control samples.

In order to generate ROC curves for the entire data set, a software tool on the site https://www.metaboanalyst.ca/MetaboAnalyst/upload/RocUploadView.xhtml was used. A list of 853 proteins where there was sufficient expression across the sample set to generate high confidence in the results was used as an analytical data set. Many of the 1440 proteins which were seen in less than 50% of patient samples were excluded. Using this software tool, a ROC curves for all 853 proteins in the sample set were generated.

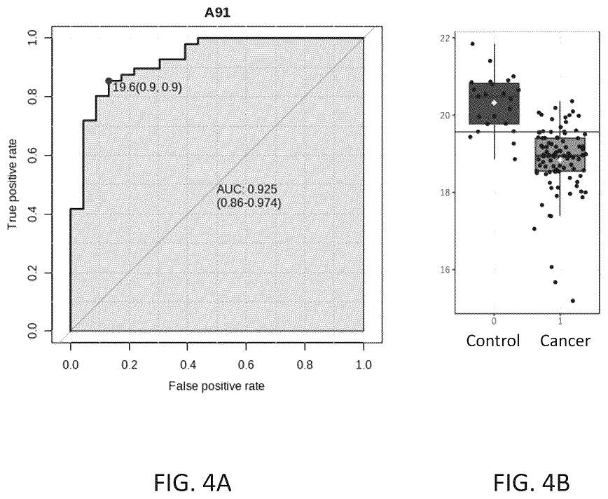

FIG. 4A shows the ROC curve and sample distribution of the top hit Heparin Cofactor 2 (“HC2”), which had the highest AUC value (0.926) out of all of the biomarkers assessed in this study. FIG. 4B shows the expression distribution of HC2, comparing expression in microparticle preparations from cancer patients (right) vs microparticle preparations from non-cancer control subject (left). The differential expression between cancer and non-cancer cohorts is significant (p-value=8.43E-12).

The top 60 cancer biomarkers, based on highest AUC values, and p-values<0.05, are listed in Table 6, starting from the biomarkers with the highest AUC value.

TABLE 2.1
The top 60 pan cancer biomarkers, based on highest AUC and p-values < 0.05

| ROC | | |
|---|---|---|
| AUC | P-value | Biomarker protein name, and corresponding gene name (GN) |
| 0.926 | 8.43E−12 | Heparin cofactor 2 GN = SERPIND1 |
| 0.921 | 3.55E−09 | cDNA FLJ53075, highly similar to Kininogen-1 |
| 0.902 | 4.76E−10 | Phosphatidylinositol-glycan-specific phospholipase D |
| | | GN = GPLD1 |
| 0.890 | 1.27E−06 | cDNA FLJ51597, highly similar to C4b-binding protein alpha |
| | | chain |
| 0.876 | 1.67E−07 | Apolipoprotein A-IV GN = APOA4 |
| 0.865 | 8.55E−04 | Vitamin K-dependent protein S GN = PROS1 |
| 0.861 | 2.01E−09 | Complement C1q subcomponent subunit A GN = C1QA |
| 0.856 | 3.01E−07 | Complement C1q subcomponent subunit B GN = C1QB |
| 0.841 | 1.07E−05 | Transthyretin GN = TTR |
| 0.839 | 3.67E−06 | Complement C3 GN = C3 |
| 0.837 | 1.05E−08 | IGL c3084_light_IGLV3-27_IGLJ2 (Fragment) |
| 0.836 | 4.47E−06 | Biotinidase GN = BTD |
| 0.835 | 5.66E−08 | Carboxypeptidase B2 GN = CPB2 |
| 0.832 | 8.04E−08 | IG c829_heavy_IGHV3-9_IGHD6-13_IGHJ4 (Fragment) |
| 0.827 | 1.13E−05 | Alpha-1-acid glycoprotein 2 GN = ORM2 |
| 0.827 | 1.74E−08 | C4b-binding protein beta chain GN = C4BPB |
| 0.826 | 2.31E−06 | Kininogen-1 GN = KNG1 |
| 0.825 | 1.68E−06 | Serum paraoxonase/arylesterase 1 GN = PON1 |
| 0.822 | 2.11E−08 | Hyaluronan-binding protein 2 GN = HABP2 |
| 0.820 | 1.21E−06 | Band 3 anion transport protein GN = SLC4A1 |
| 0.818 | 4.91E−07 | Plasma kallikrein GN = KLKB1 |
| 0.817 | 3.67E−05 | Alpha-2-HS-glycoprotein GN = AHSG |
| 0.811 | 7.92E−07 | Cholinesterase GN = BCHE |
| 0.804 | 1.99E−05 | IG c256_heavy_IGHV3-33_IGHD3-9_IGHJ6 (Fragment) |
| 0.803 | 2.56E−06 | Gc-globulin GN = HEL-S-51 |
| 0.802 | 9.89E−06 | IGH c2663_heavy_IGHV5-51_IGHD3-10_IGHJ4 (Fragment) |
| 0.798 | 7.70E−06 | Mannan-binding lectin serine protease 2 GN = MASP2 |
| 0.796 | 2.28E−06 | Inhibin beta E chain GN = INHBE |
| 0.796 | 5.03E−06 | IGL c323_light_IGLV7-43_IGLJ2 (Fragment) |
| 0.793 | 2.70E−05 | Extracellular matrix protein 1 GN = ECM1 |
| 0.791 | 1.15E−06 | Uncharacterized protein tr\|Q8NEJ1\|Q8NEJ1_HUMAN |
| 0.789 | 1.93E−05 | Retinol-binding protein 4 GN = RBP4 |
| 0.787 | 1.51E−06 | Coagulation factor X GN = F10 |
| 0.781 | 3.13E−05 | Fibrinogen alpha chain GN = FGA |
| 0.779 | 1.22E−04 | ACX82 (Fragment) |
| 0.777 | 7.64E−06 | IGH c13_heavy_IGHV1-18_IGHD3-10_IGHJ4 (Fragment) |
| 0.774 | 1.17E−05 | Myosin-9 GN = MYH9 |
| 0.772 | 2.24E−05 | Vitamin D binding protein (Fragment) GN = Gc |
| 0.772 | 8.20E−06 | Uncharacterized protein GN = DKFZp686K03196 |
| | | tr\|Q6N095\|Q6N095_HUMAN |
| 0.769 | 1.70E−04 | Complement C1q subcomponent subunit C GN = C1QC |
| 0.768 | 1.21E−04 | Apolipoprotein A-II GN = APOA2 |
| 0.767 | 1.49E−04 | ITIH4 protein GN = ITIH4 |
| 0.764 | 4.06E−06 | Insulin-like growth factor-binding protein 3 GN = IGFBP3 |
| 0.763 | 5.23E−05 | IGH c3886_heavy_IGHV3-15_IGHD2-15_IGHJ4 (Fragment) |
| 0.762 | 2.18E−05 | Immunoglobulin heavy chain variable region (Fragment) |
| | | tr\|A0A7T0PYL3\|A0A7T0PYL3_HUMAN |
| 0.761 | 3.34E−06 | IG c1219_light_IGLV3-25_IGLJ1 (Fragment) |
| 0.760 | 0.003803 | cDNA FLJ75416, highly similar to Homo sapiens complement |
| | | factor H (CFH), mRNA |
| 0.760 | 1.70E−04 | IGH c1399_heavy_IGHV3-33_IGHD7-27_IGHJ6 (Fragment) |
| 0.759 | 1.52E−05 | Ceruloplasmin GN = CP >tr\|A5PL27\|A5PL27_HUMAN CP |
| | | protein GN = CP |
| 0.759 | 4.69E−04 | Tenascin C GN = TNC |
| 0.757 | 1.97E−04 | Attractin GN = ATRN |
| 0.756 | 5.73E−04 | Thyroxine-binding globulin GN = SERPINA7 |
| 0.755 | 2.58E−04 | Afamin GN = AFM |
| 0.753 | 5.55E−05 | L-selectin GN = SELL |
| 0.752 | 3.03E−05 | Complement component C8 beta chain GN = C8B |
| 0.752 | 6.82E−05 | Immunoglobulin delta heavy chain sp\|P0DOX3\|IGD_HUMAN |
| 0.751 | 2.04E−05 | IGH c4066_heavy_IGHV3-74_IGHD1-26_IGHJ4 (Fragment) |
| 0.750 | 2.22E−04 | IG c1570_light_IGKV3-11_IGKJ3 (Fragment) |
| 0.746 | 4.74E−07 | Platelet factor 4 GN = PF4 |
| 0.746 | 1.32E−04 | Stomatin GN = STOM |

As can be seen, the top 60 biomarkers as single analytes range in AUC from 0.746 up to 0.926. AUCs above 0.9 represent strongly predictive biomarkers. It was also found that many of the identified biomarkers were likely to be corona proteins that are not from the source cells of the microparticles, but rather “host proteins” in the local environments that the microparticles may have resided, or have traversed, within the subject (i.e. host) after being released from the source cell, and associated with the microparticles through, e.g., protein-protein or receptor-ligand interactions. In cases where a host protein expression level reflects an overall disease state of the host, the host protein may be referred to as a “host disease response protein”.

### Differential Expression as Measured by LC/MS Quantification is Reproduced in ELISA

LC/MS platform measure peptides known to be uniquely specific to an identified protein. By contrast, immune-analysis such as with ELISA (enzyme-linked immunosorbent assay) or proximity extension assays require the presence of intact protein for signal generation. ELISA was performed to confirm that expression seen by LC/MS from patient plasma was reproducible using an orthogonal analytical platform.

For ELISA, microparticles were isolated from plasma using qEVoriginal Gen 2 35 nm columns (IZON). Plasma was centrifuged at 1,500×g for 10 min to remove cells and cellular debris. Columns were equilibrated with two column volumes of double distilled water (ddH₂O) and 500 μL of cell-free plasma was loaded to the top of the column. Once plasma had entered loading frit, columns were washed with 2.5 mL ddH₂O. Wash was discarded and columns were loaded with 400 uL ddH₂O per fraction. In total, five 400 uL microparticle-enriched (ME) fractions were collected. The five microparticle-enriched fractions were then pooled to prepare the microparticle preparations. The microparticles in the microparticle preparations were then lysed by diluting the microparticle preparation with PBS, combining 1:1 with RIPA lysis buffer containing protease inhibitor, and incubating for 5 min at RT. Following lysis, the microparticle preparation were analyzed with a commercially available ELISA kit according to the manufacturer's instructions.

FIG. 3 shows the results of an ELISA analysis of C1q of microparticle preparations from non-cancer control subjects and microparticle preparations from cancer patients. A Thermo Fisher® Human C1q ELISA Kit was used for the analysis. As seen in FIG. 3, ELISA analysis of C1q in microparticle preparations from cancer patients and non-cancer control subjects demonstrated differential expression of C1q. Comparing C1q expression in the non-cancer cohort against all the cancer cohorts together (pan cancer) shows a 1.67-fold change and a p-value of <0.001. This orthogonal protein data demonstrates that differential expression seen in LC/MS based on peptide detection is also seen in the same-patient microparticle preparations using ELISA analysis of intact proteins.

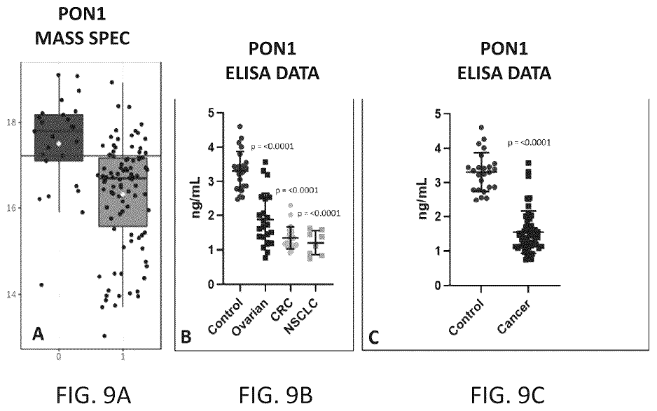

FIGS. 9A-9C shows the cancer-based differential expression of another microparticle associated protein as measured through LC/MS quantification compared with ELISA. FIG. 9A shows the expression distribution of serum paraoxonase/arylesterase 1 (gene name PON1), comparing expression in microparticle preparations from the combined cancer cohorts (right) against microparticle preparations from the non-cancer control cohort (left). FIGS. 9B-9C show the results of an ELISA analysis of the same protein, serum paraoxonase/arylesterase 1, in microparticle preparations from non-cancer control subjects compared against microparticle preparations from cancer patients. A Thermo Fisher® Human PON1 ELISA Kit was used for the analysis. FIG. 9B shows the ELISA-based quantification paraoxonase/arylesterase 1 in microparticle preparations from the different cancer cohorts separately (ovarian, CRC, and NCSLC). FIG. 9C shows the ELISA-based quantification paraoxonase/arylesterase 1 in microparticle preparations from the same cancer cohorts combined (pan cancer). As can be seen in FIGS. 9A-9C, the reduced expression of serum paraoxonase/arylesterase 1 in cancer patients as detected through LC/MS quantification (FIG. 9A) is reproduced in ELISA, in each of ovarian, CRC, and NCSLC (FIG. 9B) as well as in cancer generally (FIG. 9C).

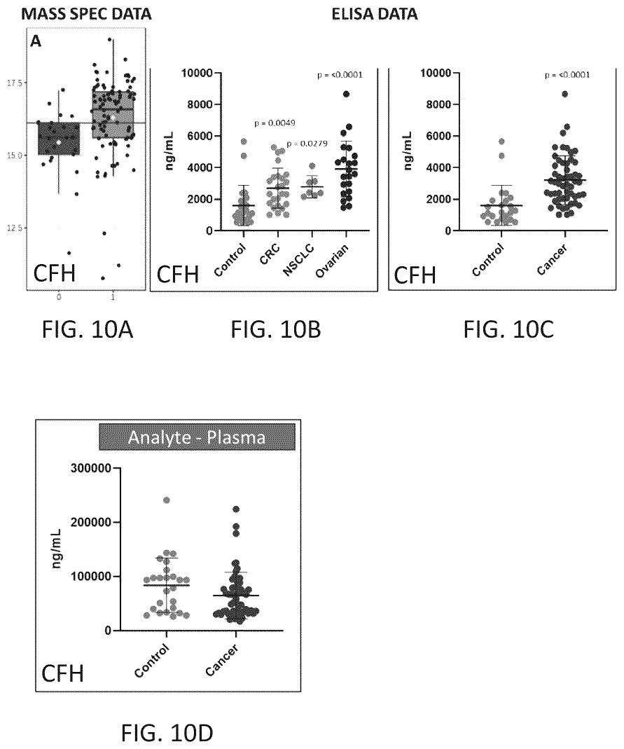

A similar effect is shown in FIGS. 10A-10C with respect to complement factor H (gene name CFH). The increased expression of complement factor H in cancer patients as detected through LC/MS quantification (FIG. 10A) is reproduced in ELISA, in each of ovarian, CRC, and NCSLC (FIG. 10B) as well as in cancer generally (FIG. 10C).

It was found that in some cases, the cancer/non-cancer differential expression of a biomarker that was observed in microparticle preparations was not observed when the same biomarker was assessed in native plasma that was not treated to isolate or enrich for microparticles, thus indicating that it was critical, at least with certain MAPs, to examine expression in the microparticle preparations, and not in native plasma. For example, FIG. 10D shows the results of an ELISA analysis of complement factor H in native plasma from non-cancer control subjects compared against native plasma from cancer patients. Under such conditions, there was no significant difference in complement factor H expression levels between the cancer and non-cancer cohorts. This result highlights the importance of the unique method of microparticle enrichment in detecting cancer with certain biomarkers that can only be observed in the enriched fractions of plasma and not in the patient's plasma without enrichment of the signals.

Determining Presence of Cancer with Multiple Biomarkers

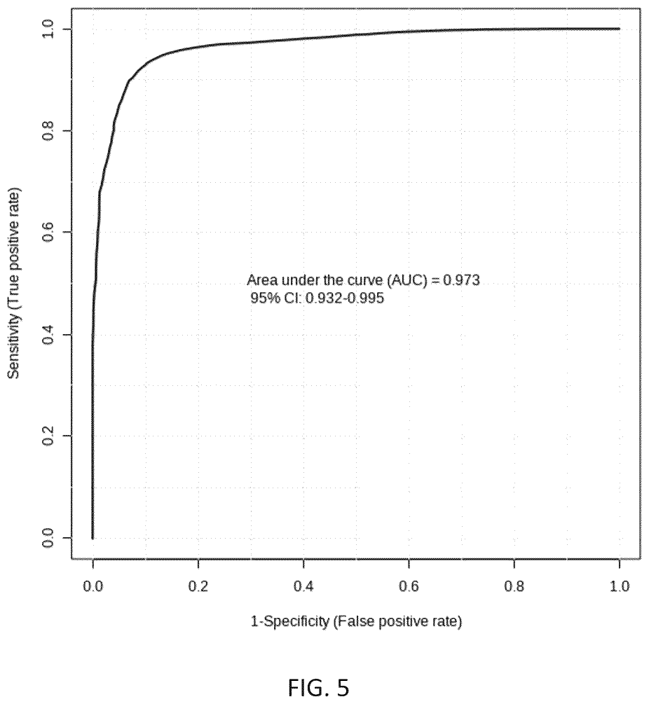

In addition to generating ROC curves for each individual biomarker, ROC curves for groups of biomarkers (“multiplexes”) were calculated using the same software. Initially, a sub-groups in which each biomarker had a functional role in different biological/physiological functions was studied. One such subgroup is listed in Table 2.2, and the ROC curve (“collective ROC curve”) for the sub-group is shown in FIG. 5. The AUC value for the sub-group listed in Table 2.2 was 0.973, which meant that the five markers collectively served to more accurately indicate, with higher confidence, the presence of cancer in the subject than any single biomarker.

TABLE 2.2
Exemplary multiplex

Heparin cofactor 2 GN = SERPIND1
Phosphatidylinositol-glycan-specific phospholipase D GN = GPLD1
Complement C1q subcomponent subunit A GN = C1QA
Biotinidase GN = BTD
Band 3 anion transport protein GN = SLC4A1

The performance of sub-groups based on the top 20 hits was determined based on AUC. As can be seen in Table 2.3, these sub groups based on the aggregate top 20 biomarkers listed in Table 2.1 (top 20, top 19, top 18, etc.) demonstrate AUCs ranging from 0.958 to 0.981.

TABLE 2.3
The AUC value of sub-groups out of the
top 20 biomarkers listed in Table 2.1

| | | 95% confidence |
|---|---|---|
| Sub-group from Table 2.1 | AUC | interval |
| Top 20 | 0.981 | 0.952-0.997 |
| Top 19 | 0.981 | 0.958-0.996 |
| Top 18 | 0.979 | 0.96-0.995 |
| Top 17 | 0.979 | 0.959-0.996 |
| Top 16 | 0.978 | 0.958-0.995 |
| Top 15 | 0.971 | 0.951-0.992 |
| Top 14 | 0.965 | 0.941-0.991 |
| Top 13 | 0.962 | 0.931-0.986 |
| Top 12 | 0.963 | 0.931-0.985 |
| Top 11 | 0.963 | 0.927-0.987 |
| Top 10 | 0.965 | 0.931-0.987 |
| Top 9 | 0.963 | 0.919-0.984 |
| Top 8 | 0.963 | 0.925-0.982 |
| Top 7 | 0.964 | 0.929-0.982 |
| Top 6 | 0.965 | 0.927-0.981 |
| Top 5 | 0.962 | 0.908-0.985 |
| Top 4 | 0.966 | 0.939-0.988 |
| Top 3 | 0.958 | 0.913-0.995 |
| Top 2 | 0.958 | 0.918-0.994 |

Taken together, these data demonstrate, that an extremely high confidence diagnostic test can be fashioned from grouping together various biomarkers in multiple different panels. Moreover, these data suggest than an extremely high confidence diagnostic test could be fashioned from, 2, 3, 4, 5, or 6 biomarkers identified in the screen described herein above.

A similar analysis was conducted for each cancer type separately, to identify strongly predictive biomarkers for each of breast cancer, colorectal cancer, lung cancer, and ovarian cancer, as described herein below.

### Example 3: Breast Cancer Biomarkers

A similar analysis was conducted with a subset of the cohort, comparing microparticle associated protein expression between microparticle preparations collected from the 25 non-cancer subjects and the 25 stage 2/3 breast cancer patients as described in Example 1, in order to identify strongly predictive biomarkers for breast cancer.

The top breast cancer biomarkers, based on highest AUC and p-values<0.05 are listed in Table 3.1.

TABLE 3.1
the top breast cancer biomarkers based on highest AUC and p-values <0.05

| AUC | P-value | Biomarker protein name, and corresponding gene name |
|---|---|---|
| 0.9104 | 1.80E−07 | Haptoglobin GN = HP |
| 0.9024 | 7.60E−08 | cDNA FLJ53075, highly similar to Kininogen-1 |
| | | tr\|B4DPP8\|B4DPP8_HUMAN |
| 0.9008 | 1.82E−07 | Phosphatidylinositol-glycan-specific phospholipase D GN = GPLD1 |
| 0.8928 | 1.95E−05 | Complement C1q subcomponent subunit A GN = C1QA |
| 0.8656 | 9.52E−05 | Heparin cofactor 2 GN = SERPIND1 |
| 0.864 | 1.96E−06 | Beta-1 metal-binding globulin tr\|B4E1B2\|B4E1B2_HUMAN |
| 0.856 | 5.87E−06 | Mannan-binding lectin serine protease 2 GN = MASP2 |
| 0.8528 | 5.67E−06 | Fibrinogen alpha chain GN = FGA |
| 0.8496 | 4.44E−06 | Complement component C9 GN = C9 |
| 0.8368 | 0.014625 | Vitamin K-dependent protein S GN = PROS1 |
| 0.8304 | 4.17E−06 | Myosin-9 GN = MYH9 |
| 0.8304 | 2.10E−05 | IGL c3084_light_IGLV3-27_IGLJ2 (Fragment) |
| 0.8256 | 1.53E−05 | Inhibin beta E chain GN = INHBE |
| 0.8224 | 0.004152 | cDNA FLJ51597, highly similar to C4b-binding protein alpha chain |
| | | tr\|B4E1D8\|B4E1D8_HUMAN |
| 0.8208 | 5.22E−05 | Apolipoprotein A-IV GN = APOA4 |
| 0.8192 | 0.001966 | Alpha-1-acid glycoprotein 2 GN = ORM2 |
| 0.8192 | 0.001119 | Apolipoprotein H (Fragment) tr\|D9IWP9\|D9IWP9_HUMAN |
| 0.8176 | 1.13E−04 | Coagulation factor X GN = F10 |
| 0.8176 | 2.00E−05 | Alpha-1-antichymotrypsin GN = SERPINA3 |
| 0.8176 | 1.11E−04 | IGH c2663_heavy_IGHV5-51_IGHD3-10_IGHJ4 (Fragment) |
| 0.816 | 5.37E−04 | Biotinidase GN = BTD |
| 0.8128 | 0.00239 | Complement C1q subcomponent subunit B GN = C1QB |
| 0.8112 | 3.41E−05 | Transforming growth factor-beta-induced protein ig-h3 GN = TGFBI |
| 0.8112 | 0.002154 | Haptoglobin (Fragment) GN = HP |
| 0.808 | 0.001956 | IG c519_light_IGKV3-15_IGKJ4 (Fragment) |
| 0.8032 | 0.001841 | Out at first protein homolog GN = OAF |
| 0.8016 | 6.40E−04 | IG c771_light_IGKV1-5_IGKJ2 (Fragment) |
| 0.8016 | 1.55E−04 | ACX82 (Fragment) tr\|A0A679KL62\|A0A679KL62_HUMAN |
| 0.7984 | 7.21E−05 | Stomatin GN = STOM |
| 0.7968 | 8.23E−05 | Band 3 anion transport protein GN = SLC4A1 |
| 0.7952 | 8.67E−05 | Scavenger receptor cysteine-rich type 1 protein M130 GN = CD163 |
| 0.7904 | 1.62E−04 | Ceruloplasmin GN = CP >tr\|A5PL27\|A5PL27_HUMAN CP protein |
| | | GN = CP |
| 0.7856 | 1.32E−04 | IG c401_heavy_IGHV1-69_IGHD5-5_IGHJ2 (Fragment) |
| 0.784 | 5.22E−04 | Alpha-2-antiplasmin GN = SERPINF2 |
| 0.784 | 0.00108 | Hyaluronan-binding protein 2 GN = HABP2 |
| 0.784 | 2.59E−04 | IG c829_heavy_IGHV3-9_IGHD6-13_IGHJ4 (Fragment) |
| 0.784 | 0.14667 | IGH c1129_heavy_IGHV1-18_IGHD3-9_IGHJ4 (Fragment) |
| 0.7824 | 0.006947 | SAA2-SAA4 readthrough GN = SAA2-SAA4 |
| 0.7824 | 0.001118 | Alpha-1B-glycoprotein GN = A1BG |
| 0.7808 | 2.31E−04 | Gc-globulin GN = HEL-S-51 |

### Example 4: Colorectal Cancer Biomarkers

A similar analysis was conducted with a subset of the cohort, comparing microparticle associated protein expression between microparticle preparations collected from the 25 non-cancer subjects and the stage 3/4 colorectal cancer patients as described in Example 1, in order to identify strongly predictive biomarkers for colorectal cancer.

The top colorectal cancer biomarkers, based on highest AUC and p-values<0.05 are listed in Table 4.1.

TABLE 4.1
the top colorectal cancer biomarkers based on highest AUC and p-values <0.05

| AUC | p-value | Biomarker protein name, and corresponding gene name |
|---|---|---|
| 0.9104 | 1.80E−07 | Haptoglobin GN = HP |
| 0.9632 | 4.26E−11 | Apolipoprotein A-IV GN = APOA4 |
| 0.9584 | 4.35E−09 | Complement C1q subcomponent subunit B GN = C1QB |
| 0.9424 | 6.72E−09 | cDNA FLJ53075, highly similar to Kininogen-1 |
| | | tr\|B4DPP8\|B4DPP8_HUMAN |
| 0.936 | 4.80E−06 | cDNA FLJ51597, highly similar to C4b-binding protein alpha chain |
| | | tr\|B4E1D8\|B4E1D8_HUMAN |
| 0.9216 | 1.84E−08 | Phosphatidylinositol-glycan-specific phospholipase D GN = GPLD1 |
| 0.9088 | 2.66E−06 | Complement C1q subcomponent subunit A GN = C1QA |
| 0.9088 | 1.73E−08 | Cholinesterase GN = BCHE |
| 0.904 | 1.41E−04 | Vitamin K-dependent protein S GN = PROS1 |
| 0.8864 | 1.59E−05 | Extracellular matrix protein 1 GN = ECM1 |
| 0.88 | 6.05E−07 | Tenascin GN = TNC |
| 0.88 | 1.67E−06 | ITIH4 protein GN = ITIH4 |
| 0.8768 | 1.10E−06 | Gc-globulin GN = HEL-S-51 |
| 0.8752 | 7.43E−07 | IGH c13_heavy_IGHV1-18_IGHD3-10_IGHJ4 (Fragment) |
| 0.8672 | 1.56E−04 | Heparin cofactor 2 GN = SERPIND1 |
| 0.8656 | 9.13E−06 | IG c86_heavy_IGHV5-51_IGHD3-16_IGHJ6 (Fragment) |
| 0.864 | 1.59E−06 | Afamin GN = AFM |
| 0.864 | 9.34E−06 | IG c829_heavy_IGHV3-9_IGHD6-13_IGHJ4 (Fragment) |
| 0.8624 | 5.41E−06 | Lumican GN = LUM >tr\|A0A384N669\|A0A384N669_HUMAN Lumican |
| 0.856 | 9.06E−07 | Carboxypeptidase B2 GN = CPB2 |
| 0.8496 | 3.32E−05 | Complement C1q subcomponent subunit C GN = C1QC |
| 0.848 | 0.002048 | Transthyretin GN = TTR |
| 0.8448 | 7.81E−06 | Vitamin D binding protein (Fragment) GN = Gc |
| 0.8432 | 2.02E−05 | Thyroxine-binding globulin GN = SERPINA7 |
| 0.84 | 2.95E−05 | Plasma kallikrein GN = KLKB1 |
| 0.84 | 1.97E−04 | cDNA FLJ75416, highly similar to Homo sapiens complement factor H |
| | | (CFH), mRNA |
| 0.8368 | 9.60E−06 | IGL c3084_light_IGLV3-27_IGLJ2 (Fragment) |
| 0.8304 | 1.02E−05 | Hyaluronan-binding protein 2 GN = HABP2 |
| 0.8224 | 4.61E−05 | C4b-binding protein beta chain GN = C4BPB |
| 0.8192 | 6.55E−05 | Fibronectin GN = FN1 |
| 0.816 | 5.27E−05 | Mannan-binding lectin serine protease 2 GN = MASP2 |
| 0.8128 | 1.74E−04 | IGL c323_light_IGLV7-43_IGLJ2 (Fragment) |
| 0.8112 | 4.81E−05 | Myosin-9 GN = MYH9 |
| 0.8112 | 0.06758 | Complement C3 GN = C3 |
| 0.8112 | 5.22E−05 | Complement factor H GN = CFH |
| 0.8064 | 0.009797 | Kininogen-1 GN = KNG1 |
| 0.7968 | 0.002508 | Gelsolin GN = GSN |
| 0.7936 | 4.93E−04 | Serum paraoxonase/arylesterase 1 GN = PON1 |
| 0.7936 | 9.82E−05 | IGL c1742_light_IGKV3-20_IGKJ4 (Fragment) |
| 0.7936 | 0.001099 | IGH c4066_heavy_IGHV3-74_IGHD1-26_IGHJ4 (Fragment) |
| 0.792 | 3.82E−04 | Insulin-like growth factor-binding protein 3 GN = IGFBP3 |

### Example 5: Lung Cancer Biomarkers

A similar analysis was conducted with a subset of the cohort, comparing exosome associated protein expression between microparticle preparations collected from the 25 non-cancer subjects and the 19 non small-cell lung cancer patients as described in Example 1, in order to identify strongly predictive biomarkers for lung cancer.

The top lung cancer biomarkers, based on highest AUC and p-values<0.05, are listed in Table 5.1.

TABLE 5.1
the top lung cancer biomarkers based on highest AUC and p-values <0.05

| AUC | P-value | Biomarker protein name, and corresponding gene name |
|---|---|---|
| 0.97474 | 1.58E−10 | cDNA FLJ53075, highly similar to Kininogen-1 |
| | | tr\|B4DPP8\|B4DPP8_HUMAN |
| 0.92211 | 1.97E−07 | Phosphatidylinositol-glycan-specific phospholipase D GN = GPLD1 |
| 0.92211 | 5.02E−07 | Plasma kallikrein GN = KLKB1 |
| 0.90947 | 4.78E−04 | cDNA FLJ51597, highly similar to C4b-binding protein alpha chain |
| | | tr\|B4E1D8\|B4E1D8_HUMAN |
| 0.86737 | 0.038895 | Complement C3 GN = C3 |
| 0.86737 | 4.46E−05 | Thyroxine-binding globulin GN = SERPINA7 |
| 0.86526 | 1.46E−05 | IGH c2663_heavy_IGHV5-51_IGHD3-10_IGHJ4 (Fragment) |
| 0.86105 | 8.00E−04 | Heparin cofactor 2 GN = SERPIND1 |
| 0.85684 | 9.46E−06 | Gc-globulin GN = HEL-S-51 |
| 0.85474 | 0.0052 | Vitamin K-dependent protein S GN = PROS1 |
| 0.85263 | 1.40E−05 | Vitamin D binding protein (Fragment) GN = Gc |
| 0.85263 | 6.58E−05 | IGL c323_light_IGLV7-43_IGLJ2 (Fragment) |
| 0.84842 | 1.82E−05 | Apolipoprotein A-IV GN = APOA4 |
| 0.84421 | 1.75E−05 | Cholinesterase GN = BCHE |
| 0.83579 | 4.09E−04 | Insulin-like growth factor-binding protein 3 GN = IGFBP3 |
| 0.83579 | 3.90E−05 | Carboxypeptidase B2 GN = CPB2 |
| 0.82947 | 4.43E−04 | Hyaluronan-binding protein 2 GN = HABP2 |
| 0.82737 | 2.62E−04 | Selenoprotein P GN = SELENOP |
| 0.82526 | 0.00808 | Kininogen-1 GN = KNG1 |
| 0.82526 | 0.001098 | PRO2275 tr\|Q9P173\|Q9P173_HUMAN |
| 0.82105 | 0.001529 | Complement C1q subcomponent subunit B GN = C1QB |
| 0.81474 | 1.86E−04 | Tenascin GN = TNC |
| 0.81263 | 2.59E−04 | IG c599_heavy_IGHV3-53_IGHD4-4_IGHJ4 (Fragment) |
| 0.81263 | 4.45E−04 | IGH c3220_heavy_IGHV3-49_IGHD2-15_IGHJ3 (Fragment) |
| 0.81053 | 8.25E−04 | IGH c1338_heavy_IGHV3-48_IGHD2-21_IGHJ4 (Fragment) |
| 0.80632 | 3.45E−04 | C4b-binding protein beta chain GN = C4BPB |
| 0.80421 | 5.59E−04 | Complement component C8 beta chain GN = C8B |
| 0.80421 | 3.54E−04 | IG c256_heavy_IGHV3-33_IGHD3-9_IGHJ6 (Fragment) |
| 0.80421 | 0.002204 | Apolipoprotein C-IV GN = APOC4 |
| 0.80211 | 4.99E−04 | Pregnancy zone protein GN = PZP |
| 0.79579 | 4.71E−04 | Apolipoprotein H (Fragment) tr\|D9IWP9\|D9IWP9_HUMAN |
| 0.79368 | 0.001258 | Serum paraoxonase/arylesterase 1 GN = PON1 |
| 0.79368 | 0.001201 | IG c829_heavy_IGHV3-9_IGHD6-13_IGHJ4 (Fragment) |
| 0.79368 | 4.01E−04 | IGH c13_heavy_IGHV1-18_IGHD3-10_IGHJ4 (Fragment) |
| 0.79368 | 7.99E−04 | Alpha-1B-glycoprotein GN = A1BG >tr\|V9HWD8\|V9HWD8_HUMAN |
| | | Epididymis secretory sperm binding protein Li 163pA GN = HEL-S-163pA |
| 0.78947 | 5.29E−04 | Ceruloplasmin GN = CP >tr\|A5PL27\|A5PL27_HUMAN CP protein |
| | | GN = CP |
| 0.78737 | 5.12E−04 | Inhibin beta E chain GN = INHBE |
| 0.78737 | 8.66E−05 | Actin, alpha skeletal muscle GN = ACTA1 |
| 0.78737 | 8.55E−04 | Complement factor H GN = CFH |
| 0.78526 | 0.00356 | Complement C1q subcomponent subunit A GN = C1QA |

### Example 6: Ovarian Cancer Biomarkers

A similar analysis was conducted with a subset of the cohort, comparing microparticle associated protein expression between microparticle preparations collected from the 25 non-cancer subjects and the 25 stage 3/4 ovarian cancer patients as described in Example 1, in order to identify strongly predictive biomarkers for ovarian cancer.

The top ovarian cancer biomarkers, based on highest AUC and p-values<0.05 are listed in Table 6.1.

TABLE 6.1
the top ovarian cancer biomarkers based on highest AUC and p-values < 0.05

| AUC | P-value | Biomarker protein name, and corresponding gene name |
|---|---|---|
| 0.9664 | 4.70E−06 | cDNA FLJ51597, highly similar to C4b-binding protein alpha |
| | | chain tr\|B4E1D8\|B4E1D8_HUMAN |
| 0.9536 | 3.70E−07 | cDNA FLJ53075, highly similar to Kininogen-1 |
| | | tr\|B4DPP8\|B4DPP8_HUMAN |
| 0.9456 | 1.51E−09 | Phosphatidylinositol-glycan-specific phospholipase D |
| | | GN = GPLD1 |
| 0.9408 | 3.69E−08 | Apolipoprotein A-IV GN = APOA4 |
| 0.904 | 6.46E−07 | Complement C1q subcomponent subunit B GN = C1QB |
| 0.9008 | 1.11E−07 | IGH c3142_heavy_IGHV3-33_IGHD3-3_IGHJ3 (Fragment) |
| 0.8992 | 1.56E−06 | Hyaluronan-binding protein 2 GN = HABP2 |
| 0.8976 | 5.24E−08 | Uncharacterized protein tr\|Q8NEJ1\|Q8NEJ1_HUMAN |
| 0.888 | 8.11E−04 | Vitamin K-dependent protein S GN = PROS1 |
| 0.8784 | 1.16E−04 | Bone marrow proteoglycan GN = PRG2 |
| 0.8768 | 1.21E−06 | Cholinesterase GN = BCHE |
| 0.872 | 2.29E−05 | Complement C1q subcomponent subunit A GN = C1QA |
| 0.8704 | 1.37E−04 | Complement factor H-related protein 4 GN = CFHR4 |
| 0.8608 | 2.96E−06 | Plasma kallikrein GN = KLKB1 |
| 0.8592 | 3.47E−06 | C4b-binding protein beta chain GN = C4BPB |
| 0.8512 | 6.21E−06 | FGA protein GN = FGA |
| 0.848 | 1.85E−06 | Inhibin beta E chain GN = INHBE |
| 0.848 | 4.22E−06 | Carboxypeptidase B2 GN = CPB2 |
| 0.8464 | 3.45E−06 | IGL c323_light_IGLV7-43_IGLJ2 (Fragment) |
| 0.8448 | 2.97E−04 | Heparin cofactor 2 GN = SERPIND1 |
| 0.8432 | 1.15E−04 | ACX82 (Fragment) tr\|A0A679KL62\|A0A679KL62_HUMAN |
| 0.8416 | 2.89E−05 | Attractin GN = ATRN |
| 0.84 | 3.12E−06 | Vascular cell adhesion protein 1 GN = VCAM1 |
| 0.84 | 7.80E−06 | Polymeric immunoglobulin receptor GN = PIGR |
| 0.8384 | 1.91E−05 | IGH c2663_heavy_IGHV5-51_IGHD3-10_IGHJ4 (Fragment) |
| 0.8352 | 3.00E−04 | Biotinidase GN = BTD |
| 0.8336 | 1.00E−04 | IGL c1787_light_IGKV1D-17_IGKJ2 (Fragment) |
| 0.8336 | 1.63E−04 | IGH c164_heavy_{——}IGHV3-11_IGHD1-26_IGHJ3 (Fragment) |
| 0.8336 | 3.28E−05 | IG c829_heavy_IGHV3-9_IGHD6-13_IGHJ4 (Fragment) |
| 0.8304 | 1.05E−04 | Protein AMBP GN = AMBP |
| 0.8288 | 8.62E−05 | Plexin domain-containing protein 2 GN = PLXDC2 |
| 0.8272 | 1.10E−05 | IGL c3084_light_IGLV3-27_IGLJ2 (Fragment) |
| 0.8272 | 3.45E−05 | Complement C1q subcomponent subunit C GN = C1QC |
| 0.8256 | 0.003123 | Transthyretin GN = TTR |
| 0.8256 | 1.66E−05 | Gc-globulin GN = HEL-S-51 |
| 0.8192 | 0.004675 | Kininogen-1 GN = KNG1 |
| 0.8192 | 1.05E−04 | Band 3 anion transport protein GN = SLC4A1 |
| 0.8192 | 9.71E−06 | Uncharacterized protein GN = DKFZp686K03196 |
| 0.8176 | 4.65E−05 | Noelin GN = OLFM1 |
| 0.816 | 0.064213 | Complement C3 GN = C3 |

### Example 7—Further MS Analysis of Ovarian Cancer Microparticles

The initial pan cancer analysis of MAPs was extended with a deeper analysis of ovarian cancer microparticles with a repeat DIA analysis using the Biognosys® True Discovery Mass Spectrometry Proteomics Platform, and identified 645 proteins that were differentially expressed in microparticle preparations from the cancer cohort compared to microparticle preparations from the non-cancer control cohort.

Microparticle-enriched fractions of plasma samples were prepared from the 25 stage 3/4 ovarian cancer patients and 25 non-cancer control subjects as described above in Example 1. The microparticle-associated proteins were then extracted and digested as described above in Example 1, prepared for MS evaluation and peptide quantification based on specifications for the Biognosys® True Discovery Mass Spectrometry Proteomics Platform, then evaluated and quantified with the Biognosys® True Discovery Mass Spectrometry Proteomics Platform. The quantification data was then analyzed in Data Independent Acquisition (DIA) mode as described above in Example 1 (e.g. in the “Bioinformatic Analysis” section).

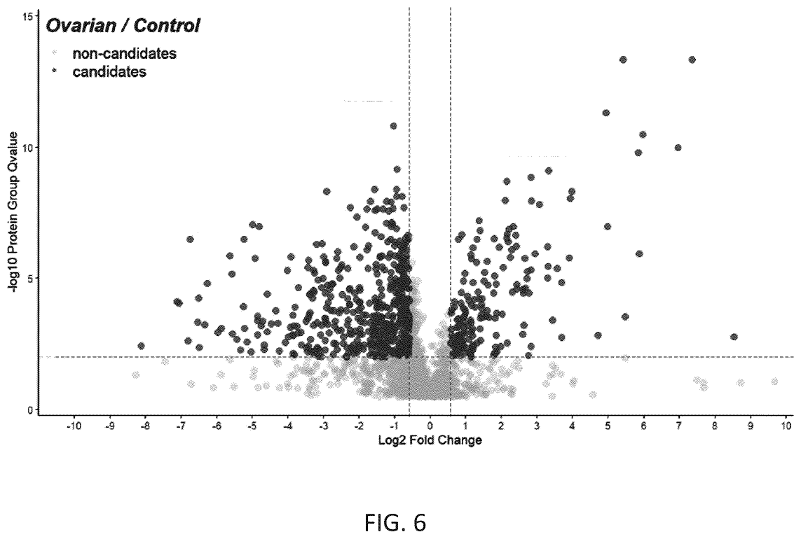

FIG. 6 shows a Volcano Plot of differentially expressed proteins in microparticle preparations from ovarian cancer patients compared with non-cancer control subjects. The X-axis represents fold change of protein and the Y-axis represents the p-value of protein. A total of 645 out of 1929 total unique proteins had cancer/non-cancer differential expression that met both cutoff criteria of fold change (log 2FC>0.58) and statistical significance (p-value<0.01).

Out of the 645 differentially expressed proteins, the top 25 ovarian cancer biomarkers, based on lowest q-value (an adjusted p-value, adjusted using a Storey and Tibshirani approach), along with Log2FC were selected. These biomarkers are listed in Table 7.1.

TABLE 7.1
the top ovarian cancer biomarkers based on lowest q-values and Log2FC

| | | | Biomarker protein name, and corresponding gene |
|---|---|---|---|
| # | Log2FC | q-value | name (GN) |
| 1 | 5.425 | 4.56E−14 | AE1 (anion exchanger 1) GN = SLC4A1 |
| 2 | 7.357 | 4.56E−14 | Ankyrin-1 GN = ANK1 |
| 3 | 4.946 | 4.98E−12 | Beta-spectrin GN = SPTB |
| 4 | −1.024 | 1.60E−11 | SNED1 (Sushi, Nidogen, and EGF-like Domains 1) |
| | | | GN = SNED1 |
| 5 | 5.975 | 3.27E−11 | Erythrocyte membrane protein band 4.1 GN = EPB41 |
| 6 | 6.964 | 1.06E−10 | Erythrocyte membrane protein band 4.2 GN = EPB42 |
| 7 | 5.849 | 1.66E−10 | Alpha-spectrin GN = SPTA1 |
| 8 | −0.931 | 6.95E−10 | UDP-GlcNAc:betaGal beta-1,3-N- |
| | | | acetylglucosaminyltransferase 2 GN = B3GNT2 |
| 9 | 3.328 | 8.20E−10 | Hematopoietic proteoglycan core protein GN = SRGN |
| 10 | 2.828 | 1.44E−09 | Amyloid precursor protein GN = APP |
| 11 | 2.158 | 2.01E−09 | Ribonuclease 4 GN = RNASE4 |
| 12 | −1.573 | 4.12E−09 | ADAM like decysin 1 GN = ADAMDEC1 |
| 13 | −0.940 | 4.12E−09 | Bone morphogenetic protein 1 GN = BMP1 |
| 14 | 3.978 | 4.90E−09 | Vascular endothelial growth factor receptor 1 |
| | | | (VEGFR1) GN = FLT |
| 15 | −2.911 | 4.90E−09 | GN = FAM234A |
| 16 | −0.787 | 7.64E−09 | coagulation factor X GN = F10 |
| 17 | −0.945 | 7.64E−09 | endoglin GN = ENG |
| 18 | 3.942 | 8.93E−09 | Platelet factor 4 GN = PF4 |
| 19 | 2.115 | 1.08E−08 | Factor XI GN = F11 |
| 20 | 2.851 | 1.13E−08 | protein myosin-9 GN = MYH9 |
| 21 | −1.678 | 1.20E−08 | alpha-1,6-mannosylglycoprotein 6-beta-N- |
| | | | acetylglucosaminyltransferase GN = MGAT5 |
| 22 | −1.232 | 1.20E−08 | mucosal vascular addressin cell adhesion molecule 1 |
| | | | GN = MADCAM1 |
| 23 | −1.101 | 1.26E−08 | fibulin 7 GN = FBLN7 |
| 24 | 3.082 | 1.58E−08 | latelet Factor 4 Variant 1 GN = PF4V1 |
| 25 | −0.736 | 1.98E−08 | TGF beta induced or βig-h3 GN = TGFBI |

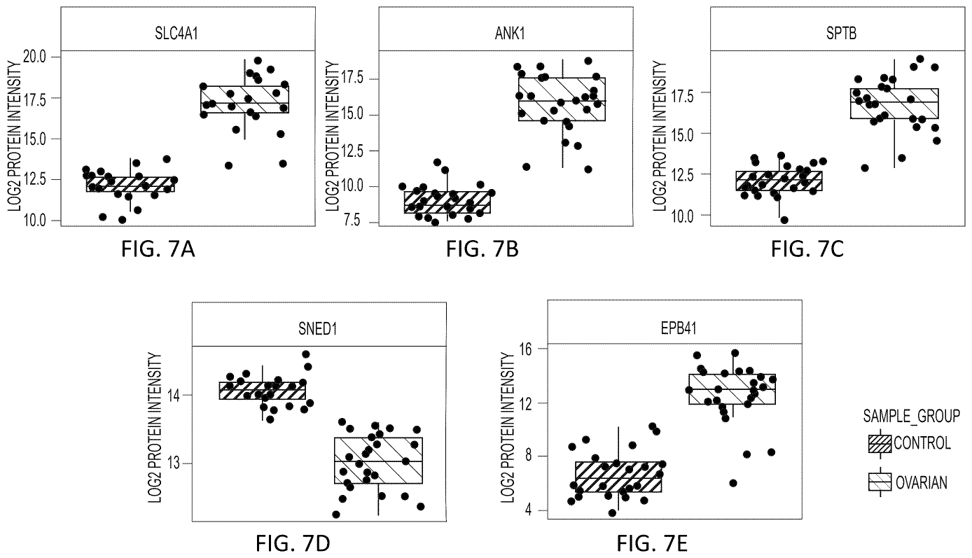

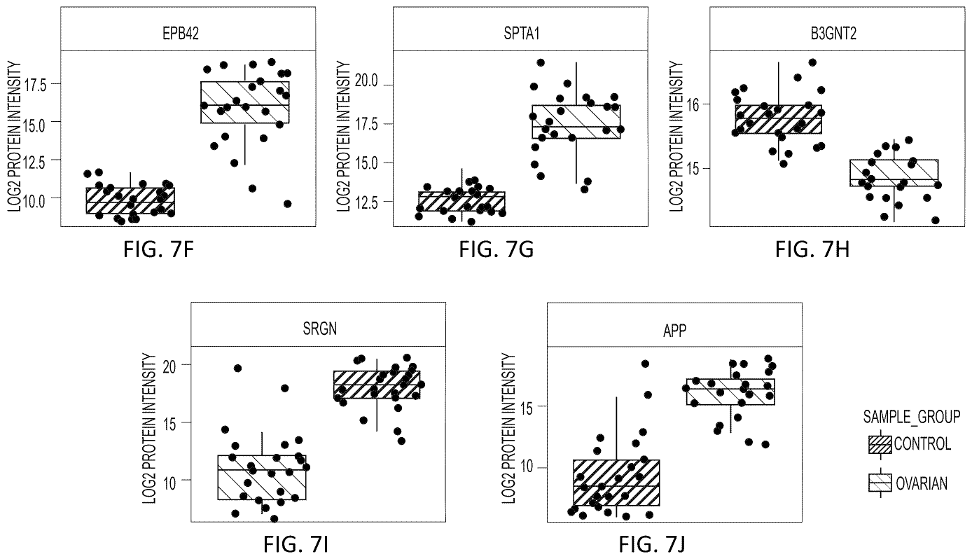

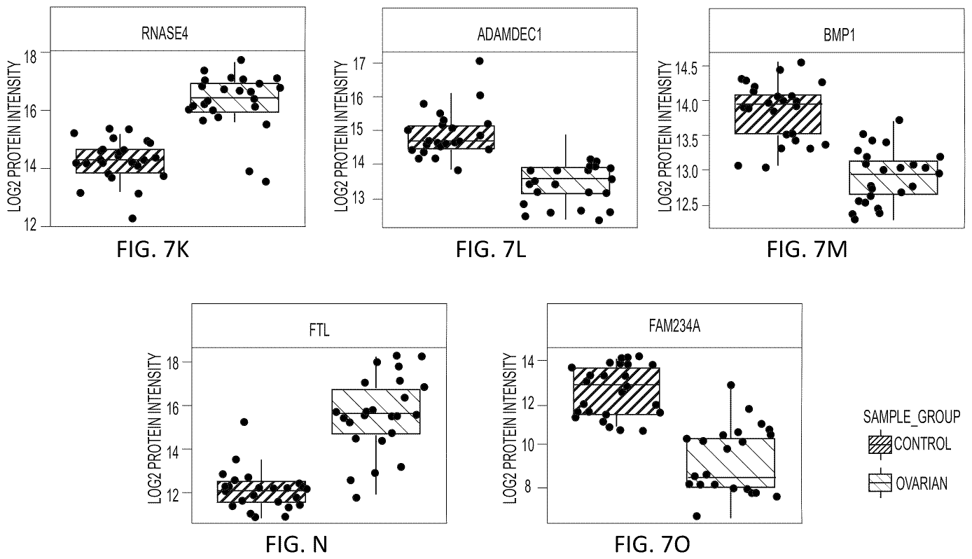

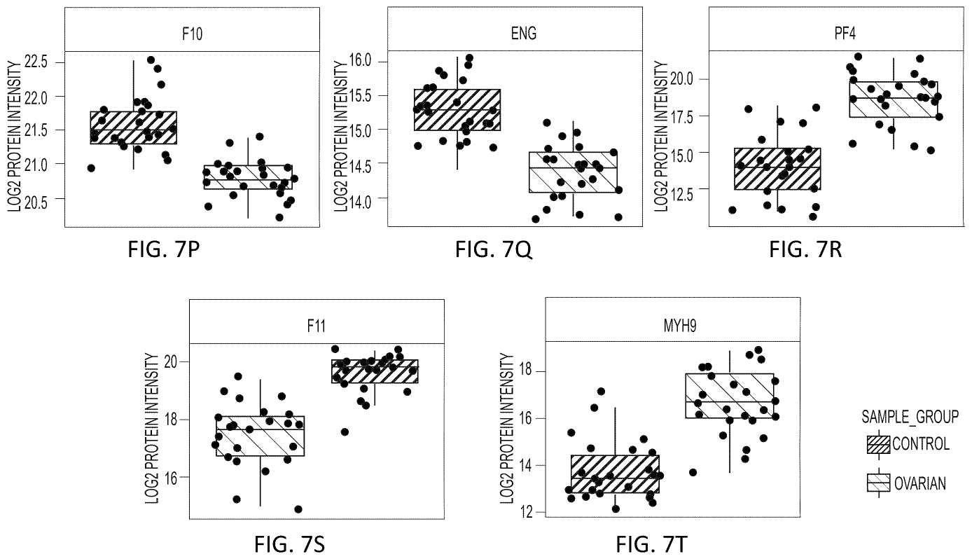

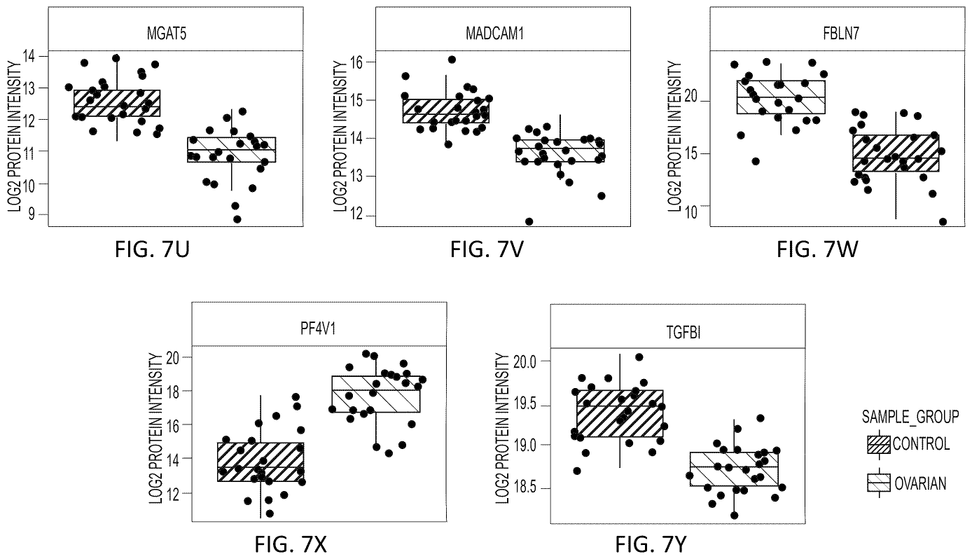

The ovarian cancer biomarkers listed in Table 7.1 represent a wide variety of biomarkers, including proteins not typically associated with cancer screening and diagnosis, including immune, metabolic and inflammatory proteins. FIGS. 7A-7Y shows the expression distribution of the ovarian cancer biomarkers listed in Table 7.1 above, comparing expression in microparticle preparations from patients in the ovarian cancer cohort (right) with expression in microparticle preparations from the non-cancer control cohort (left).

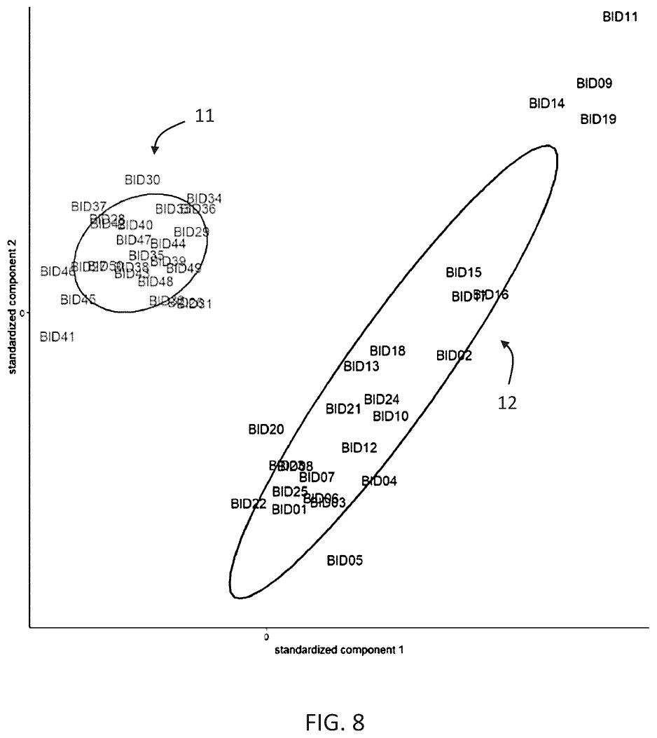

FIG. 8 shows a Partial Least Squares discriminant (PLS-DA) analysis of the differential expression of biomarkers in Ovarian cancer samples (upper left cluster 11) vs non-cancer samples (lower right cluster 12). The plot shows component 1 (x axis) plotted against component 2 (y axis), grouped by sample group. Component 1 represents the difference between samples from Ovarian and Control groups. Component 2 represents the difference between individual samples from Control group. Each “BID” represents a particular sample.

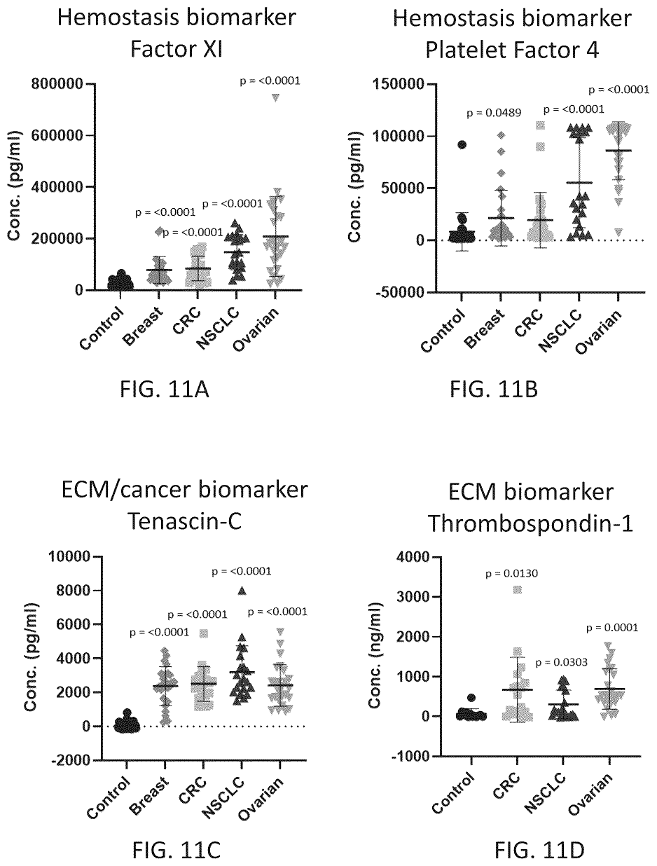

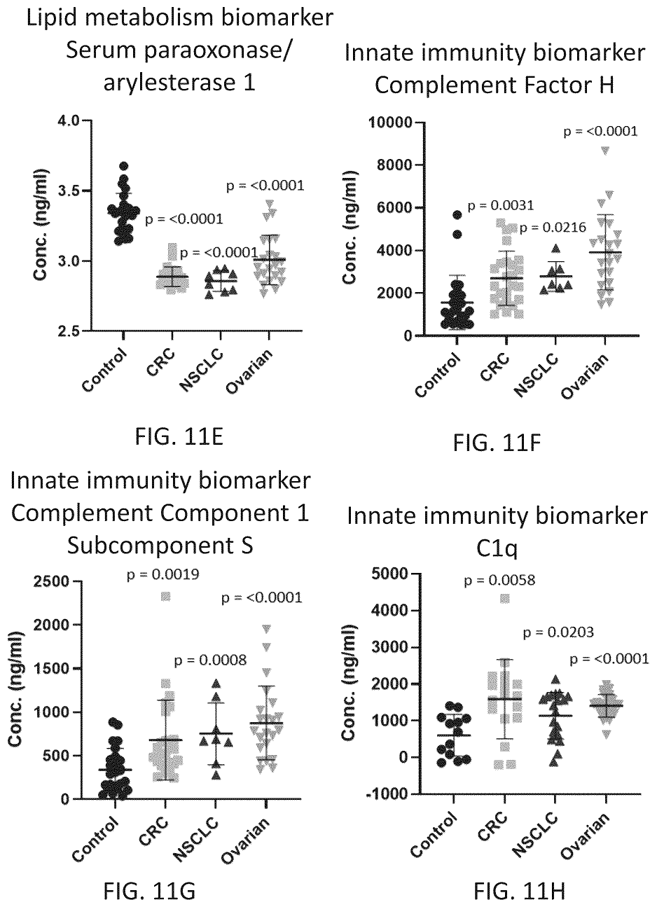

The cancer/non-cancer differential expression as detected by MS of each of the 25 ovarian cancer biomarkers listed in Table 7.1, as well as other biomarkers also shown to have significant differential expression in the MS analysis, was recapitulated in ELISA. FIGS. 11A-11H show a selection of the results from the ELISA analysis, which includes the following biomarkers:

FIG. 11A: Factor XI (encoded by the F11 gene);

FIG. 11B: Platelet Factor 4 (encoded by the PF4 gene);

FIG. 11C: Tenascin-C (encoded by the TNC gene);

FIG. 11D: Thrombospondin-1 (encoded by the THBS1 gene);

FIG. 11E: Serum paraoxonase/arylesterase 1 (encoded by the PON1 gene);

FIG. 11F: Complement Factor H (encoded by the CFH gene);

FIG. 11G: Complement Component 1 Subcomponent S (encoded by the Cls gene); and

FIG. 11H: Complement Component 1q (C1q)

The ELISA analysis was not only performed with microparticle preparations from ovarian cancer to confirm the differential expression detected through MS, but was also performed with microparticle preparations from breast cancer, CRC, and lung cancer. As such, FIGS. 11A-11H shows that many of the top 25 ovarian cancer biomarkers listed in Table 7.1 are also capable of serving as biomarkers that detect not only ovarian cancer, but also one or more of breast cancer, CRC, and lung cancer.

### Biomarker Selection Based on Random Forest Modeling and Recursive Feature Elimination (RFE)

An alternative selection of “top” ovarian cancer biomarkers from the MS data set was performed utilizing machine learning methods. In particular, random forest modeling and recursive feature elimination was used to select biomarkers based on the MS data set described above. A random forest model iteratively builds decision trees by selecting random subsets of features and data points. During this process, it calculates the importance of each feature by measuring how much the tree nodes using that feature reduce impurity. Features with higher impurity reduction are considered more important and thus selected for inclusion in the final feature set. Recursive Feature Elimination (RFE) systematically removes less important features by recursively training a model and ranking features based on their contribution to model performance. The process continues until the desired number of features remains or until a specified performance metric is optimized.

All data analyses for the random forest model and RFE were carried out using Rstudio 2023.12.1 and Python 3.11.8 version. Raw intensities from the MS data set were normalized using the log 2 method, and a constant was added to avoid negative values for further downstream analysis. Principal component analysis (PCA), Uniform manifold Approximation and Projection (UMAP) and Partial least squares discriminant analysis (PLS-DA) was used for dimensionality reduction and visualization. Feature (biomarker) selection was undertaken by random forest (RF) modelling using the Scikit-leam package in Python language. Random forests (using a random seed set at 42) from a stratified bootstrap selection to get same proportion of cancer and control in training and test set (approximately 70% for a randomly selected training set and 30% as test set). The model was initially tuned to obtain the best hyperparameters (max features and n estimators) using grid search stratified cross validation (5-fold, 100 number of repeats) on training set. max features parameter decides the number of features to consider when looking for the best split of the decision trees. n estimators parameter decides the number of decision trees on which an ensemble is built. Best hyperparameter defined on the training set was then used for prediction on the test set, from which the final accuracy metrics were derived. The most discriminatory proteins were ranked according to a feature importance score based on their based on their contribution to the Mean Decrease Impurity (Gini importance) of the RF algorithm, and designated as RF-identified biomarkers.

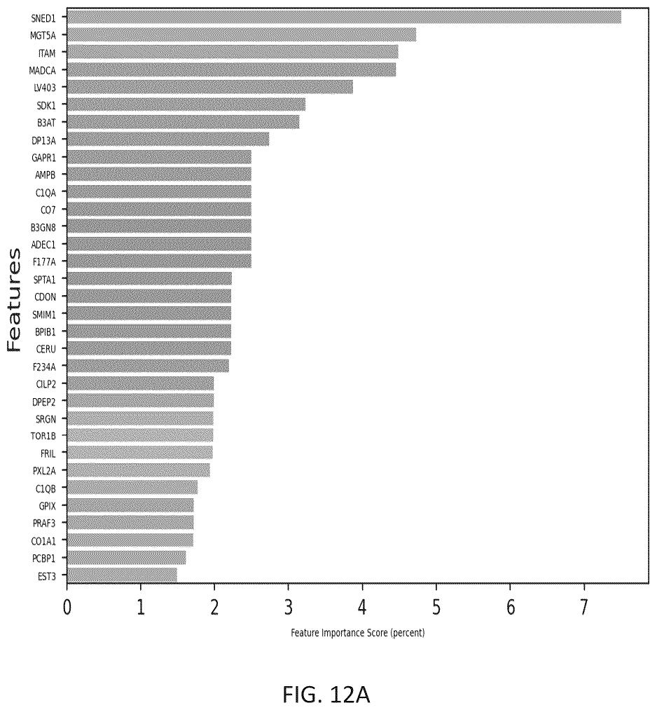

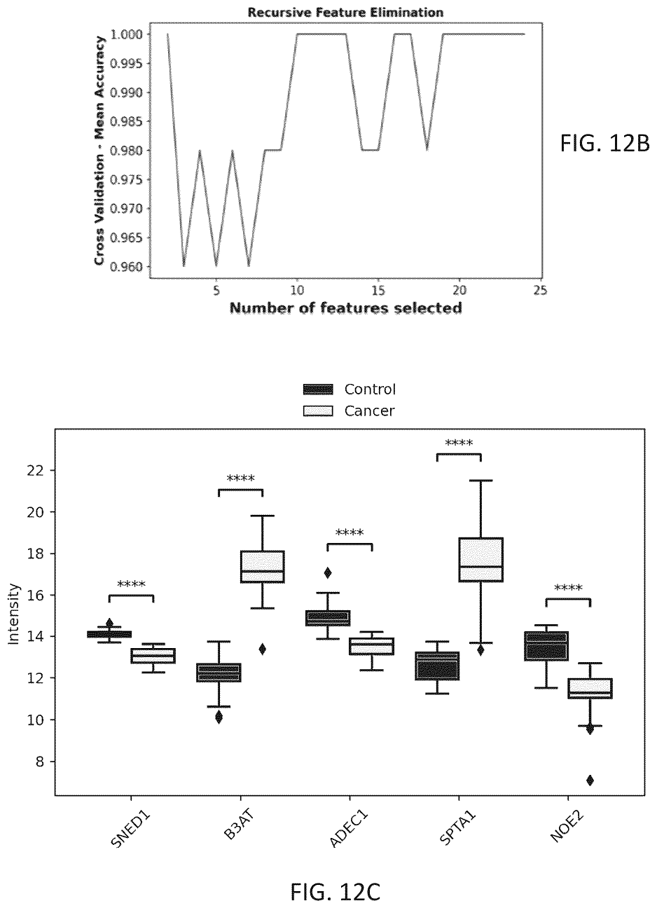

In a first analysis, random forest modeling was used to identify a set of biomarkers from the MS data set of 1929 total unique proteins, which resulted in a set of 63 ovarian cancer biomarkers that demonstrated robust differential expression between ovarian cancer and non-cancer control cohorts, and received a high feature importance score. A visualization of the ranking of the RF-identified biomarkers having a feature importance score of over 1 is shown in FIG. 12A, and the 63 RF-identified ovarian cancer biomarkers are provided in Table 7.2 herein below, listed in order of their respective feature importance scores. Each row is one of the biomarkers, each of which is identified by a respective UniprotKB (Uniprot Knowledgebase) unique protein entry name (column 1; “Protein”), UniprotKB unique accession number (column 2; “Uniprot AN”), and a colloquial protein name (column 3; “Protein name”.

TABLE 7.2
RF-identified ovarian cancer biomarkers

| # | Protein | Uniprot AN | Protein Name |
|---|---|---|---|
| 1 | SNED1 | Q8TER0 | Sushi, nidogen and EGF-like domain- |
| | | | containing protein 1 |
| 2 | MGT5A | Q09328 | Alpha-1,6-mannosylglycoprotein 6-beta-N- |
| | | | acetylglucosaminyltransferase A |
| 3 | ITAM | P11215 | Integrin alpha-M |
| 4 | MADCA | Q13477 | Mucosal addressin cell adhesion molecule 1 |
| 5 | LV403 | A0A075B6K6 | Immunoglobulin lambda variable 4-3 |
| 6 | SDK1 | Q7Z5N4 | Protein sidekick-1 |
| 7 | B3AT | P02730 | Band 3 anion transport protein |
| 8 | DP13A | Q9UKG1 | DCC-interacting protein 13-alpha |
| 9 | GAPR1 | Q9H4G4 | Golgi-associated plant pathogenesis-related |
| | | | protein 1 |
| 10 | AMPB | Q9H4A4 | Aminopeptidase B |
| 11 | C1QA | P02745 | Complement C1q subcomponent subunit A |
| 12 | CO7 | P10643 | Complement component C7 |
| 13 | B3GN8 | Q7Z7M8 | UDP-GlcNAc:betaGal beta-1,3-N- |
| | | | acetylglucosaminyltransferase 8 |
| 14 | ADEC1 | O15204 | ADAM DEC1 |
| 15 | F177A | Q8N128 | Protein FAM177A1 |
| 16 | SPTA1 | P02549 | Spectrin alpha chain, erythrocytic 1 |
| 17 | CDON | Q4KMG0 | Cell adhesion molecule-related/down- |
| | | | regulated by oncogenes |
| 18 | SMIM1 | B2RUZ4 | Small integral membrane protein 1 |
| 19 | BPIB1 | Q8TDL5 | BPI fold-containing family B member 1 |
| 20 | CERU | P00450 | Ceruloplasmin |
| 21 | F234A | Q9H0X4 | Protein FAM234A |
| 22 | CILP2 | Q8IUL8 | Cartilage intermediate layer protein 2 |
| 23 | DPEP2 | Q9H4A9 | Dipeptidase 2 |
| 24 | SRGN | P10124 | Serglycin |
| 25 | TOR1B | O14657 | Torsin-1B |
| 26 | FRIL | P02792 | Ferritin light chain |
| 27 | PXL2A | Q9BRX8 | Peroxiredoxin-like 2A |
| 28 | C1QB | P02746 | Complement C1q subcomponent subunit B |
| 29 | GPIX | P14770 | Platelet glycoprotein IX |
| 30 | PRAF3 | O75915 | PRA1 family protein 3 |
| 31 | CO1A1 | P02452 | Collagen alpha-1(I) chain |
| 32 | PCBP1 | Q15365 | Poly(rC)-binding protein 1 |
| 33 | EST3 | Q6UWW8 | Carboxylesterase 3 |
| 34 | PCSK9 | Q8NBP7 | Proprotein convertase subtilisin/kexin type 9 |
| 35 | PIGR | P01833 | Polymeric immunoglobulin receptor |
| 36 | MFGM | Q08431 | Lactadherin |
| 37 | AXDN1 | Q5T1B0 | Axonemal dynein light chain domain- |
| | | | containing protein 1 |
| 38 | QSOX1 | O00391 | Sulfhydryl oxidase 1 |
| 39 | AMPL | P28838 | Cytosol aminopeptidase |
| 40 | GPNMB | Q14956 | Transmembrane glycoprotein NMB |
| 41 | PRELP | P51888 | Prolargin |
| 42 | ITB1 | P05556 | Integrin beta-1 |
| 43 | NOE2 | O95897 | Noelin-2 |
| 44 | ADHX | P11766 | Alcohol dehydrogenase class-3 |
| 45 | TRXR1 | Q16881 | Thioredoxin reductase 1, cytoplasmic |
| 46 | KV108 | A0A0C4DH67 | Immunoglobulin kappa variable 1-8 |
| 47 | TM223 | A0PJW6 | Transmembrane protein 223 |
| 48 | HEP2 | P05546 | Heparin cofactor 2 |
| 49 | IPSP | P05154 | Plasma serine protease inhibitor |
| 50 | EF2 | P13639 | Elongation factor 2 |
| 51 | PFKAL | P17858 | ATP-dependent 6-phosphofructokinase, liver |
| | | | type |
| 52 | BLM | P54132 | RecQ-like DNA helicase BLM |
| 53 | TREA | O43280 | Trehalase |
| 54 | HD | P42858 | Huntingtin |
| 55 | NAR3 | Q13508 | Ecto-ADP-ribosyltransferase 3 |
| 56 | PPIA | P62937 | Peptidyl-prolyl cis-trans isomerase A |
| 57 | MAT1 | P51948 | CDK-activating kinase assembly factor MAT1 |
| 58 | GAS6 | Q14393 | Growth arrest-specific protein 6 |
| 59 | LV233 | A0A075B6J2 | Probable non-functional immunoglobulin |
| | | | lambda variable 2-33 |
| 60 | FBLN7 | Q53RD9 | Fibulin-7 |
| 61 | CD166 | Q13740 | CD166 antigen |
| 62 | DCD | P81605 | Dermcidin |
| 63 | FA11 | P03951 | Coagulation factor XI |

Feature selection using the Recursive Feature Elimination (RFE) cross validation algorithm was then performed on the RF-identified biomarkers. RFE was run using Scikit-learn package in Python language to identify the most accurate biomarkers for multiplex (n=5) biomarker development. Differences between study groups were assessed using t test for continuous variables and applied a false discovery rate adjustment for multiple testing using the Benjamini-Hochberg correction method. A visualization of the RFE cross validation is shown in FIG. 12B.

A rotating selection of biomarker subsets of the RFE-cross validated biomarkers were tested for predictive value based on p-value and ROC curve AUC. Each of the RFE-cross validated biomarkers demonstrated cancer/non-cancer differential expression with an adjusted p-value<10e-4, and each subset had extremely high predictive accuracy, having a combined ROC curve with an AUC of 1, which signifies perfect predictive accuracy. An example set of a highly predictive 5-plex as determined by RFE is the 5-plex of the SNED1, B3AT, ADEC1, SPTA1, and NOE2 proteins. FIG. 12C shows the expression distribution those proteins as quantified by MS, comparing expression in microparticle preparations from the ovarian cancer (“Cancer”) cohort (gray; right) against microparticle preparations from the non-cancer (“Control”) cohort (black; left). Diamond plots indicate outliers not included in the analysis. As shown in the figure, each of the five biomarkers have differential expression between ovarian cancer and non-cancer control cohorts with an adjusted p-value<10e-4 (indicated as **** in the figure).

In a second analysis, random forest modeling was used to identify a set of biomarkers from the MS data set of the 645 proteins that had cancer/non-cancer differential expression that met both cutoff criteria of fold change (log 2FC>0.58) and statistical significance (p-value<0.01). This second RF analysis resulted in a second set of RF-identified ovarian cancer biomarkers, listed in Table 7.3, that demonstrated robust differential expression between ovarian cancer and non-cancer control cohorts, and received a high feature importance score. Each row is one of the biomarkers, each of which is identified by a respective UniprotKB (Uniprot Knowledgebase) unique protein entry name (column 1; “Protein”), UniprotKB unique accession number (column 2; “Uniprot AN”), and a colloquial protein name (column 3; “Protein name”).

TABLE 7.3
Alternative RF-identified ovarian cancer biomarkers

| # | Protein | Uniprot AN | Protein Name |
|---|---|---|---|
| 1 | PLXB2 | O15031 | Plexin-B2 |
| 2 | FA11 | P03951 | Coagulation factor XI |
| 3 | RNAS4 | P34096 | Ribonuclease 4 |
| 4 | PCOC1 | Q15113 | Procollagen C-endopeptidase |
| | | | enhancer 1 |
| 5 | LV403 | A0A075B6K6 | Immunoglobulin lambda variable 4-3 |
| 6 | CHRD | Q9H2X0 | Chordin |
| 7 | SPTB1 | P11277 | Spectrin beta chain, erythrocytic |
| 8 | NOE2 | O95897 | Noelin-2 |
| 9 | MUC1 | P15941 | Mucin-1 |
| 10 | CPNE1 | Q99829 | Copine-1 |
| 11 | FRIL | P02792 | Ferritin light chain |
| 12 | BMP1 | P13497 | Bone morphogenetic protein 1 |
| 13 | CNTN6 | Q9UQ52 | Contactin-6 |
| 14 | ARF4 | P18085 | ADP-ribosylation factor 4 |
| 15 | HV316 | A0A0C4DH30 | Probable non-functional |
| | | | immunoglobulin heavy variable 3-16 |
| 16 | F177A | Q8N128 | Protein FAM177A1 |
| 17 | B3GN8 | Q7Z7M8 | UDP-GlcNAc:betaGal beta-1,3-N- |
| | | | acetylglucosaminyltransferase 8 |
| 18 | ANK1 | P16157 | Ankyrin-1 |
| 19 | ADEC1 | O15204 | ADAM DEC1 |
| 20 | AGRE5 | P48960 | Adhesion G protein-coupled receptor E5 |
| 21 | CO4A | P0C0L4 | Complement C4-A |
| 22 | MRC2 | Q9UBG0 | C-type mannose receptor 2 |
| 23 | SNED1 | Q8TER0 | Sushi, nidogen and EGF-like domain- |
| | | | containing protein 1 |
| 24 | DP13A | Q9UKG1 | DCC-interacting protein 13-alpha |
| 25 | HYAL1 | Q12794 | Hyaluronidase-1 |
| 26 | CD109 | Q6YHK3 | CD109 antigen |
| 27 | SPTA1 | P02549 | Spectrin alpha chain, erythrocytic 1 |
| 28 | MADCA | Q13477 | Mucosal addressin cell adhesion |
| | | | molecule 1 |
| 29 | SEM4B | Q9NPR2 | Semaphorin-4B |
| 30 | NDK3 | Q13232 | Nucleoside diphosphate kinase 3 |
| 31 | ITA11 | Q9UKX5 | Integrin alpha-11 |
| 32 | CLC11 | Q9Y240 | C-type lectin domain family 11 |
| | | | member A |
| 33 | BGH3 | Q15582 | Transforming growth factor-beta- |
| | | | induced protein ig-h3 |
| 34 | BTD | P43251 | Biotinidase |
| 35 | FCGRN | P55899 | IgG receptor FcRn large subunit p51 |
| 36 | SRGN | P10124 | Serglycin |
| 37 | CREL2 | Q6UXH1 | Protein disulfide isomerase CRELD2 |
| 38 | HEXA | P06865 | Beta-hexosaminidase subunit alpha |
| 39 | ANGL8 | Q6UXH0 | Angiopoietin-like protein 8 |
| 40 | FHR1 | Q03591 | Complement factor H-related protein 1 |
| 41 | FHAD1 | B1AJZ9 | Forkhead-associated domain-containing |
| | | | protein 1 |
| 42 | EPB41 | P11171 | Protein 4.1 |
| 43 | ATS13 | Q76LX8 | A disintegrin and metalloproteinase |
| | | | with thrombospondin motifs 13 |
| 44 | PCDGK | Q9UN70 | Protocadherin gamma-C3 |
| 45 | MYH9 | P35579 | Myosin-9 |
| 46 | LIRA2 | Q8N149 | Leukocyte immunoglobulin-like receptor |
| | | | subfamily A member 2 |
| 47 | CAD13 | P55290 | Cadherin-13 |
| 48 | GANAB | Q14697 | Neutral alpha-glucosidase AB |
| 49 | IBP6 | P24592 | Insulin-like growth factor-binding |
| | | | protein 6 |
| 50 | GSH0 | P48507 | Glutamate--cysteine ligase regulatory |
| | | | subunit |
| 51 | TSP4 | P35443 | Thrombospondin-4 |
| 52 | MUC18 | P43121 | Cell surface glycoprotein MUC18 |
| 53 | SIL1 | Q9H173 | Nucleotide exchange factor SIL1 |
| 54 | LRRF1 | Q32MZ4 | Leucine-rich repeat flightless- |
| | | | interacting protein 1 |
| 55 | ERAP2 | Q6P179 | Endoplasmic reticulum aminopeptidase 2 |
| 56 | NCAM2 | O15394 | Neural cell adhesion molecule 2 |
| 57 | LOX15 | P16050 | Polyunsaturated fatty acid |
| | | | lipoxygenase ALOX15 |
| 58 | HEP2 | P05546 | Heparin cofactor 2 |
| 59 | CD34 | P28906 | Hematopoietic progenitor cell antigen |
| | | | CD34 |
| 60 | CDON | Q4KMG0 | Cell adhesion molecule-related/down- |
| | | | regulated by oncogenes |
| 61 | PHLD | P80108 | Phosphatidylinositol-glycan-specific |
| | | | phospholipase D |
| 62 | LV746 | A0A075B619 | Immunoglobulin lambda variable 7-46 |
| 63 | MMRN2 | Q9H8L6 | Multimerin-2 |
| 64 | PLF4 | P02776 | Platelet factor 4 |
| 65 | CO6 | P13671 | Complement component C6 |
| 66 | CD248 | Q9HCU0 | Endosialin |
| 67 | TFR1 | P02786 | Transferrin receptor protein 1 |
| 68 | KPCB | P05771 | Protein kinase C beta type |
| 69 | CHSTC | Q9NRB3 | Carbohydrate sulfotransferase 12 |
| 70 | TENN | Q9UQP3 | Tenascin-N |
| 71 | NOE1 | Q99784 | Noelin |
| 72 | POSTN | Q15063 | Periostin |
| 73 | GGT3; GGT1 | A6NGU5; P19440 | Putative glutathione hydrolase 3 |
| | | | proenzyme; Glutathione hydrolase 1 |
| | | | proenzyme |
| 74 | FIBA | P02671 | Fibrinogen alpha chain |
| 75 | MADD | Q8WXG6 | MAP kinase-activating death domain |
| | | | protein |
| 76 | JAM1 | Q9Y624 | Junctional adhesion molecule A |
| 77 | LYAM2 | P16581 | E-selectin |
| 78 | RET4 | P02753 | Retinol-binding protein 4 |
| 79 | LTBP1 | Q14766 | Latent-transforming growth factor |
| | | | beta-binding protein 1 |
| 80 | MMRN1 | Q13201 | Multimerin-1 |
| 81 | RACK1 | P63244 | Small ribosomal subunit protein |
| | | | RACK1 |
| 82 | LBP | P18428 | Lipopolysaccharide-binding protein |
| 83 | ML12B; ML12A | O14950; P19105 | Myosin regulatory light chain |
| | | | 12B; Myosin regulatory light chain 12A |
| 84 | IGF1 | P05019 | Insulin-like growth factor I |
| 85 | PVR | P15151 | Poliovirus receptor |
| 86 | SDK2 | Q58EX2 | Protein sidekick-2 |
| 87 | KPCD | Q05655 | Protein kinase C delta type |
| 88 | R4RL2 | Q86UN3 | Reticulon-4 receptor-like 2 |
| 89 | STOM | P27105 | Stomatin |
| 90 | CEL2A | P08217 | Chymotrypsin-like elastase family |
| | | | member 2A |
| 91 | EPCR | Q9UNN8 | Endothelial protein C receptor |
| 92 | PLDX1 | Q8IUK5 | Plexin domain-containing protein 1 |
| 93 | PABP1; PABP3 | P11940; Q9H361 | Polyadenylate-binding protein |
| | | | 1; Polyadenylate-binding protein 3 |
| 94 | GPIX | P14770 | Platelet glycoprotein IX |
| 95 | NCF2 | P19878 | Neutrophil cytosol factor 2 |
| 96 | MA1C1 | Q9NR34 | Mannosyl-oligosaccharide 1,2-alpha- |
| | | | mannosidase IC |
| 97 | LV327 | P01718 | Immunoglobulin lambda variable 3-27 |
| 98 | ADHX | P11766 | Alcohol dehydrogenase class-3 |
| 99 | EGLN | P17813 | Endoglin |
| 100 | SEM4D | Q92854 | Semaphorin-4D |
| 101 | PLSL | P13796 | Plastin-2 |
| 102 | C1QC | P02747 | Complement C1q subcomponent |
| | | | subunit C |
| 103 | GLGB | Q04446 | 1,4-alpha-glucan-branching enzyme |
| 104 | FSCN1 | Q16658 | Fascin |
| 105 | ITAM | P11215 | Integrin alpha-M |
| 106 | TOR3A | Q9H497 | Torsin-3A |
| 107 | EST1 | P23141 | Liver carboxylesterase 1 |
| 108 | MMP14 | P50281 | Matrix metalloproteinase-14 |
| 109 | CADH1 | P12830 | Cadherin-1 |
| 110 | TREA | O43280 | Trehalase |
| 111 | CEMIP | Q8WUJ3 | Cell migration-inducing and hyaluronan- |
| | | | binding protein |
| 112 | XPO2 | P55060 | Exportin-2 |
| 113 | CHST3 | Q7LGC8 | Carbohydrate sulfotransferase 3 |
| 114 | SEPR | Q12884 | Prolyl endopeptidase FAP |
| 115 | OTUB1 | Q96FW1 | Ubiquitin thioesterase OTUB1 |
| 116 | RB27B | O00194 | Ras-related protein Rab-27B |
| 117 | HEM2 | P13716 | Delta-aminolevulinic acid dehydratase |
| 118 | EMIL1 | Q9Y6C2 | EMILIN-1 |
| 119 | HBG2 | P69892 | Hemoglobin subunit gamma-2 |
| 120 | SPB6 | P35237 | Serpin B6 |
| 121 | MYL9 | P24844 | Myosin regulatory light polypeptide 9 |
| 122 | HD | P42858 | Huntingtin |
| 123 | NELL2 | Q99435 | Protein kinase C-binding protein NELL2 |
| 124 | PDIA1 | P07237 | Protein disulfide-isomerase |
| 125 | CEAM6 | P40199 | Carcinoembryonic antigen-related cell |
| | | | adhesion molecule 6 |
| 126 | F13A | P00488 | Coagulation factor XIII A chain |
| 127 | MEGF8 | Q7Z7M0 | Multiple epidermal growth factor-like |
| | | | domains protein 8 |
| 128 | PKDCC | Q504Y2 | Extracellular tyrosine-protein kinase |
| | | | PKDCC |
| 129 | CLH1 | Q00610 | Clathrin heavy chain 1 |
| 130 | ALMS1 | Q8TCU4 | Centrosome-associated protein ALMS1 |
| 131 | PSA1 | P25786 | Proteasome subunit alpha type-1 |
| 132 | BGLR | P08236 | Beta-glucuronidase |
| 133 | ITB3 | P05106 | Integrin beta-3 |
| 134 | LRP1 | Q07954 | Prolow-density lipoprotein receptor- |
| | | | related protein 1 |
| 135 | B3GN2 | Q9NY97 | N-acetyllactosaminide beta-1,3-N- |
| | | | acetylglucosaminyltransferase 2 |
| 136 | CD44 | P16070 | CD44 antigen |
| 137 | PI16 | Q6UXB8 | Peptidase inhibitor 16 |
| 138 | ENTP5 | O75356 | Nucleoside diphosphate phosphatase |
| | | | ENTPD5 |
| 139 | LCAT | P04180 | Phosphatidylcholine-sterol |
| | | | acyltransferase |

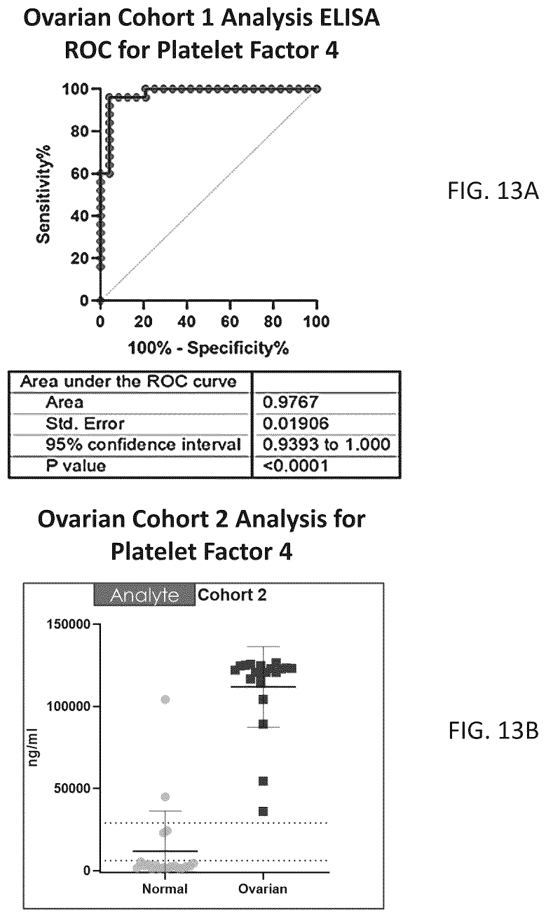

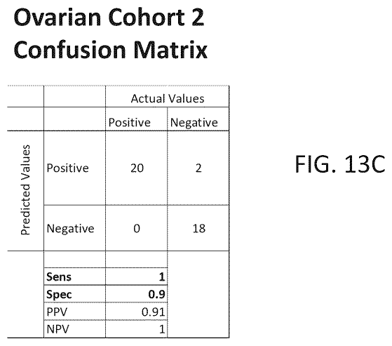

An example biomarker from the second RF-identified biomarker set is Platelet Factor 4 (encoded by the PF4 gene). ELISA analysis (with a Thermo Fisher R Human PF4 ELISA Kit) was performed with microparticle preparations samples from the same ovarian cancer cohort (cohort 1) used for the MS-based biomarker selection to confirm that the differential expression is recapitulated in an immune-assay. As shown in FIG. 13A, the ROC curve based on the ELISA quantification had an AUC of 0.988, indicating very high predictive accuracy. In addition, as shown in FIGS. 13B-13C another ELISA analysis was performed with a microparticle preparations samples from a second cohort (cohort 2) of 20 stage 3/4 ovarian cancer patients for blinded prospective cutoff testing, which demonstrated that ovarian cancer can be detected using ELISA with Platelet Factor 4 as a biomarker with extremely high sensitivity (sensitivity=1) and specificity (specificity=0.9). FIG. 13B shows the expression distribution of Platelet Factor 4 based on the ELISA quantification. The upper dotted line is a cutoff value 29149 ng/ml for platelet factor 4 ELISA quantification that was used determine sensitivity and specificity. As can be seen in FIG. 13B, 2 out of 20 normal, noncancer control subjects had a platelet factor 4 expression level above the cutoff value, and 0 out of 20 ovarian cancer patients had a platelet factor 4 expression level before the cutoff value. A confusion matrix of the results shown in FIG. 13B is provided in FIG. 13C. As shown in the confusion matrix, cancer prediction based on platelet factor 4 ELISA quantification resulted in a Positive predictive value (PPV) of 0.91, and a negative predictive value (NPV) of 1.

### Biomarkers for Cancer-Based Immunomodulation

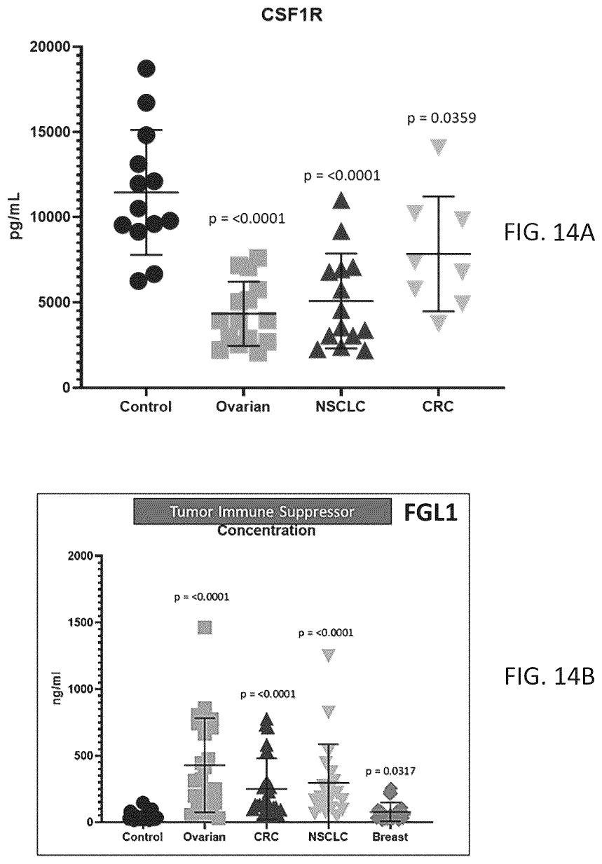

Certain biomarkers from cancer patient microparticles indicate that it is possible to track host immunosuppressive environment via antigen presenting cell (APC) biomarkers, e.g., CSF1-R (colony stimulating factor 1 receptor), and tumor immune suppressors, e.g., FGL1 (Fibrinogen-like protein 1). FIGS. 14A-14B shows the results of an ELISA analysis (using the appropriate Thermo Fisher® ELISA kits) of CSF1-R and FGL1, respectively, in microparticle preparations from non-cancer control subjects compared against microparticle preparations from a variety of cancer cohorts (ovarian cancer, NSCLC, and CRC). FIG. 14A shows that APC biomarker CSF1-R is downregulated in each of the cancer cohorts compared to the non-cancer cohort, indicating a reduction in APCs and thus a reduced immune targeting of tumors. FIG. 14B shows that tumor immune suppressor FGL1 is upregulated in each of the cancer cohorts, especially in ovarian cancer, CRC, and NSCLC, compared to the non-cancer cohort, indicating an increase.

Notably, the cancer/non-cancer differential expression of both CSF1-R and FGL1 were seen only when these biomarkers are evaluated in microparticle preparations. When the same biomarkers were evaluated (also with ELISA) in native plasma that was not treated to isolate or enrich for microparticles, no significant differences were seen, highlighting the importance of evaluating the microparticle-associated portions of these and other biomarkers, rather than their general plasma concentrations.

### Example 8—Further MS Analysis of Cohort 1 Pan Cancer Samples

Additional bioinformatics pipelines were developed in house and applied to the mass spectroscopy-based MAP differential expression data obtained from the 119 patient plasma samples (25 non-cancer control subjects, 25 stage 3/4 ovarian cancer patients, 25 stage 2/3 breast cancer patients, 25 stage 3/4 colorectal patients, and 19 stage 3/4 non small-cell lung cancer patients) described in Example 1. This MAP differential expression dataset obtained from the 119 patient plasma samples as described in Example 1 may be referred to as the “Pan Cancer dataset”.

All data analyses were carried out using Rstudio 2024.04.0 and Python 3.11.8 version. The Pan Cancer dataset has 119 samples with 94 cancer and 25 controls. The dataset was manually curated through a quality control (QC) process to exclude low-quality or likely-invalid data and duplicated proteins. For example, the QC process involved replacing outliers (visualized in boxplot) with a missing value. Missing values were replaced by very low values (ranging from 1e-4 to 1e-6), as these are likely a result of proteins being at low concentrations below the detection limit. In addition, QC was carried out to remove low intensity proteins, and only proteins with valid intensities (>1000 and not missing) in more than 50% of the samples were kept. In addition, computationally created proteins that were inferred from the peptide fragment quantification by MS, but were not associated with known proteins, were excluded. QC methods and results are reported in table 8.1. Raw intensities were normalized using log 2 method, a constant was added to avoid infinite values for further downstream analysis. As shown in the table, the dataset started with 1447 total proteins. After removal of computationally created proteins and duplicate protein lines, the dataset was pruned to 396 proteins. A curated dataset of 396 proteins was used for subsequent machine learning-based analysis, as described below.

TABLE 8.1
Pre-analysis protein curation

| | Pan Cancer | |
|---|---|---|
| Total Samples | 119 | |
| Total Proteins | 1447 | |
| Removed computationally | 416 | remaining |
| created proteins | | |
| Removed Duplicate lines | 396 | remaining |
| Outliers replaced with NA | 12 | |

The first round of Machine learning involved looking for significant proteins. Logistic regression 5-Fold 10 Repeats Stratified Cross validation using Scikit-leam package and t test with Benjamini Hochberg correction using Scipy stats package was applied on each protein. Cross-validation approach (5-fold) was used to estimate the mean ROC AUC of the model on test dataset. In 5-fold cross-validation all data is randomly split into 5 folds, then the model is trained on the 4 folds, while one fold is used as test dataset. Stratified cross validation is used to preserve the percentage of samples for each class. A two-tailed t test with equal variance was employed in all cases, an exception of Welch t test was used with unequal group variance. Proteins that have Logistic regression average AUC value greater than 0.5 and FDR corrected p-value less than 0.05 were considered significant. Machine learning parameter ‘sample weight’ was used to address any phenotype imbalance. ‘sample weight’ parameter assign higher weights to the minority class, allowing the model to pay more attention to its patterns and reducing bias towards the majority class. Proteins thus identified were considered as useful for classifying cancer vs normal (non-cancer) samples across all cancers included but not limited to the cancers included in the analysis, namely ovarian cancer, breast cancer, colorectal cancer, and non small-cell lung cancer. 61 proteins were identified through this method, and are listed in Table 8.2. In Table 8.2, each row is one of the 61 proteins, each of which is identified by a respective UniprotKB (Uniprot Knowledgebase) unique protein entry name (column 1; “Protein”), UniprotKB unique accession number (column 2; “Uniprot AN”), and a colloquial protein name (column 3; “Protein Name”). Note that the “_HUMAN” suffix was omitted from each of the protein entry names in column 1, for clarity of presentation. Column 4 shows a p-value denoting statistical significance. Column 5 shows a q-value, which is an adjusted p-value using Benjamini-Hochberg correction. Column 6 shows log 2FC, indicating the scale and direction of differential expression in Log2 units, where a negative value indicates downregulation in the cancer cohorts compared to the non-cancer cohort and a positive value indicates upregulation in the cancer cohorts compared to the non-cancer cohort. Column 7 shows the area under the curve (AUC) of the ROC curve generated from the quantification data for each protein.

TABLE 8.2
Pan cancer biomarkers

| Protein | Uniprot AN | Protein Name | p-value | q-value | log2FC | AUC |
|---|---|---|---|---|---|---|
| APOL1 | O14791 | Apolipoprotein L1 | 1.39E−04 | 1.49E−03 | −0.489 | 0.722 |
| AQR | O60306 | RNA helicase aquarius | 2.16E−04 | 2.12E−03 | 5.232 | 0.739 |
| CERU | P00450 | Ceruloplasmin | 7.24E−07 | 2.43E−05 | −0.597 | 0.751 |
| THRB | P00734 | Prothrombin | 1.19E−02 | 4.59E−02 | 0.494 | 0.648 |
| FA9 | P00740 | Coagulation factor IX | 1.91E−03 | 1.10E−02 | −0.458 | 0.725 |
| FA10 | P00742 | Coagulation factor X | 4.21E−06 | 9.89E−05 | −0.704 | 0.772 |
| ANT3 | P01008 | Antithrombin-III | 1.16E−02 | 4.54E−02 | −0.443 | 0.701 |
| CO3 | P01024 | Complement C3 | 1.88E−03 | 1.10E−02 | −0.584 | 0.806 |
| KNG1 | P01042 | Kininogen-1 | 9.66E−05 | 1.20E−03 | −0.578 | 0.800 |
| APOA2 | P02652 | Apolipoprotein A-II | 4.64E−03 | 2.37E−02 | −0.713 | 0.729 |
| FIBA | P02671 | Fibrinogen alpha chain | 1.78E−05 | 3.79E−04 | 0.632 | 0.766 |
| FIBB | P02675 | Fibrinogen beta chain | 1.85E−03 | 1.10E−02 | 0.630 | 0.681 |
| B3AT | P02730 | Band 3 anion transport | 4.19E−04 | 3.79E−03 | 4.601 | 0.840 |
| | | protein | | | | |
| C1QA | P02745 | Complement C1q | 5.38E−05 | 7.91E−04 | 1.474 | 0.859 |
| | | subcomponent subunit A | | | | |
| C1QB | P02746 | Complement C1q | 6.87E−03 | 3.12E−02 | 2.311 | 0.894 |
| | | subcomponent subunit B | | | | |
| C1QC | P02747 | Complement C1q | 2.73E−04 | 2.56E−03 | 2.257 | 0.792 |
| | | subcomponent subunit C | | | | |
| CO9 | P02748 | Complement component C9 | 1.39E−03 | 9.11E−03 | −0.451 | 0.692 |
| AMBP | P02760 | Protein AMBP | 5.98E−04 | 4.84E−03 | −0.491 | 0.754 |
| TTHY | P02766 | Transthyretin | 1.44E−03 | 9.14E−03 | −0.828 | 0.796 |
| ALBU | P02768 | Albumin | 6.48E−04 | 4.91E−03 | −0.399 | 0.661 |
| CXCL7 | P02775 | Platelet basic protein | 4.68E−04 | 3.93E−03 | 5.525 | 0.715 |
| PLF4 | P02776 | Platelet factor 4 | 5.35E−03 | 2.67E−02 | 2.999 | 0.773 |
| KLKB1 | P03952 | Plasma kallikrein | 1.45E−08 | 1.13E−06 | −0.711 | 0.846 |
| A1BG | P04217 | Alpha-1B-glycoprotein | 4.41E−04 | 3.84E−03 | −0.458 | 0.725 |
| F13B | P05160 | Coagulation factor XIII B chain | 6.46E−03 | 3.10E−02 | −0.506 | 0.665 |
| THBG | P05543 | Thyroxine-binding globulin | 8.28E−10 | 9.73E−08 | −4.579 | 0.825 |
| HEP2 | P05546 | Heparin cofactor 2 | 4.98E−08 | 2.34E−06 | −1.182 | 0.847 |
| CHLE | P06276 | Cholinesterase | 4.75E−08 | 2.34E−06 | −0.733 | 0.829 |
| APOA4 | P06727 | Apolipoprotein A-IV | 1.52E−12 | 3.58E−10 | −1.422 | 0.892 |
| PROS | P07225 | Vitamin K-dependent protein | 3.45E−07 | 1.35E−05 | 0.934 | 0.876 |
| | | S | | | | |
| CO8B | P07358 | Complement component C8 | 3.09E−05 | 5.59E−04 | −0.574 | 0.739 |
| | | beta chain | | | | |
| TSP1 | P07996 | Thrombospondin-1 | 4.34E−03 | 2.27E−02 | 4.705 | 0.704 |
| ITA2B | P08514 | Integrin alpha-IIb | 1.30E−04 | 1.45E−03 | 5.343 | 0.724 |
| APOA | P08519 | Apolipoprotein(a) | 1.30E−03 | 8.95E−03 | 1.520 | 0.695 |
| CD14 | P08571 | Monocyte differentiation | 7.69E−04 | 5.65E−03 | −0.747 | 0.622 |
| | | antigen CD14 | | | | |
| A2AP | P08697 | Alpha-2-antiplasmin | 4.34E−05 | 7.29E−04 | −0.451 | 0.749 |
| PRG2 | P13727 | Bone marrow proteoglycan | 8.86E−03 | 3.72E−02 | −3.800 | 0.685 |
| VCAM1 | P19320 | Vascular cell adhesion protein | 3.32E−06 | 8.68E−05 | −0.817 | 0.749 |
| | | 1 | | | | |
| A1AG2 | P19652 | Alpha-1-acid glycoprotein 2 | 2.09E−04 | 2.12E−03 | −0.751 | 0.778 |
| ITIH1 | P19827 | Inter-alpha-trypsin inhibitor | 8.42E−03 | 3.72E−02 | −0.373 | 0.683 |
| | | heavy chain H1 | | | | |
| PZP | P20742 | Pregnancy zone protein | 3.14E−03 | 1.72E−02 | 6.206 | 0.566 |
| C4BPB | P20851 | C4b-binding protein beta | 5.30E−05 | 7.91E−04 | 1.101 | 0.823 |
| | | chain | | | | |
| TENA | P24821 | Tenascin | 8.78E−03 | 3.72E−02 | 1.980 | 0.776 |
| STOM | P27105 | Stomatin | 1.09E−03 | 7.76E−03 | 4.013 | 0.762 |
| PROP | P27918 | Properdin | 2.01E−03 | 1.13E−02 | 0.528 | 0.650 |
| MYH9 | P35579 | Myosin-9 | 9.65E−03 | 3.92E−02 | 3.855 | 0.736 |
| K22E | P35908 | Keratin, type II cytoskeletal 2 | 8.81E−03 | 3.72E−02 | −2.175 | 0.709 |
| | | epidermal | | | | |
| AFAM | P43652 | Afamin | 3.05E−06 | 8.68E−05 | −0.768 | 0.779 |
| LUM | P51884 | Lumican | 9.23E−05 | 1.20E−03 | −0.641 | 0.763 |
| PHLD | P80108 | Phosphatidylinositol-glycan- | 9.11E−05 | 1.20E−03 | −2.009 | 0.918 |
| | | specific phospholipase D | | | | |
| HGFA | Q04756 | Hepatocyte growth factor | 1.36E−03 | 9.11E−03 | −0.842 | 0.682 |
| | | activator | | | | |
| LG3BP | Q08380 | Galectin-3-binding protein | 9.67E−03 | 3.92E−02 | 0.458 | 0.586 |
| MMRN1 | Q13201 | Multimerin-1 | 6.75E−03 | 3.12E−02 | 4.129 | 0.637 |
| HABP2 | Q14520 | Hyaluronan-binding protein 2 | 3.04E−05 | 5.59E−04 | −2.018 | 0.842 |
| LTBP1 | Q14766 | Latent-transforming growth | 6.91E−03 | 3.12E−02 | 3.904 | 0.639 |
| | | factor beta-binding protein 1 | | | | |
| ECM1 | Q16610 | Extracellular matrix protein 1 | 1.04E−04 | 1.23E−03 | −0.669 | 0.757 |
| PXDC2 | Q6UX71 | Plexin domain-containing | 1.69E−03 | 1.04E−02 | −0.308 | 0.686 |
| | | protein 2 | | | | |
| OAF | Q86UD1 | Out at first protein homolog | 1.07E−02 | 4.28E−02 | −2.988 | 0.717 |
| C163A | Q86VB7 | Scavenger receptor cysteine- | 3.96E−03 | 2.11E−02 | −3.423 | 0.742 |
| | | rich type 1 protein M130 | | | | |
| AT2A3 | Q93084 | Sarcoplasmic/endoplasmic | 5.66E−03 | 2.77E−02 | 3.844 | 0.712 |
| | | reticulum calcium ATPase 3 | | | | |
| FCGBP | Q9Y6R7 | IgGFc-binding protein | 6.33E−04 | 4.91E−03 | 0.898 | 0.762 |

The second round of Machine learning involved searching for multiplexes of 3 cancer biomarkers (“3plexes”) from the 61 pan cancer biomarkers listed in Table 8.2 that would differentiate between cancer and non-cancer MAP samples with a high degree of accuracy. Exhaustive feature selection (EFS) was performed using Linear SVM, in particular 5-fold Stratified Cross Validation. Python-based machine learning extension (MLXTEND) packages were used for this analysis. EFS is a wrapper approach for brute-force evaluation of all possible feature combinations in a specified range. In the present example, the differential expression data obtained in Example 1 from each of the cancer and non-cancer cohorts, for each of the 61 pan cancer biomarkers provided in Table 8.2, was used as training data. The training data was III divided into 5 folds, which one fold being used as a validation set and the remaining 4 folds being used as training sets for training a classifier. The training process included generation of coefficients assigned to each biomarker, with the numerical value of the coefficients becoming optimized through the training process to correctly predict cancer, compared against the known cancer or non-cancer statuses provided in the training data. For all possible 3plexes of the 61 pan cancer biomarkers, an accuracy score was calculated as a ratio of the number of instances correctly predicted by the classifier (based on the respective expression levels of a given set of 3 cancer biomarkers) to the total number of instances in the validation set. Whether the prediction of a given instance was correct was based on whether the prediction matched the known cancer or non-cancer statuses provided for the given instance in the validation set. This process was repeated for each of the 5 folds, and the respective accuracy scores averaged over the 5 folds was calculated as a “5-fold average accuracy score”. As shown in Table 8.3, the 258 3plexes with a 5-fold average accuracy score of 90% (i.e., average correct prediction ratio of 0.90) or higher were short listed.

TABLE 8.3
Pan cancer biomarker 3plexes

| | | 5-Fold |
|---|---|---|
| | | AVG |
| # | 3PLEX | Accur. |
| 1 | (CO3, C1QA, PROS) | 0.958 |
| 2 | (FA10, CO3, PROS) | 0.950 |
| 3 | (AQR, CO3, PROS) | 0.950 |
| 4 | (CO3, PROS, CO8B) | 0.950 |
| 5 | (CO3, PROS, A1AG2) | 0.942 |
| 6 | (CO3, C1QB, C4BPB) | 0.942 |
| 7 | (CO3, KNG1, PROS) | 0.941 |
| 8 | (CO3, PROS, PHLD) | 0.941 |
| 9 | (ANT3, CO3, PROS) | 0.941 |
| 10 | (CO3, PROS, VCAM1) | 0.941 |
| 11 | (CO3, PROS, MYH9) | 0.941 |
| 12 | (CO3, PROS, TSP1) | 0.941 |
| 13 | (CO3, PROS, ITA2B) | 0.941 |
| 14 | (CO3, FIBA, PROS) | 0.941 |
| 15 | (HEP2, APOA4, PROS) | 0.941 |
| 16 | (CO3, C1QB, A1AG2) | 0.933 |
| 17 | (CO3, PROS, ECM1) | 0.933 |
| 18 | (CO3, APOA2, PROS) | 0.933 |
| 19 | (CO3, F13B, PROS) | 0.933 |
| 20 | (CO3, PROS, HABP2) | 0.933 |
| 21 | (CO3, PROS, LG3BP) | 0.933 |
| 22 | (CO3, C1QA, TTHY) | 0.933 |
| 23 | (CO3, C1QA, CO9) | 0.933 |
| 24 | (CO3, B3AT, C4BPB) | 0.933 |
| 25 | (CO3, FIBB, PROS) | 0.933 |
| 26 | (CO3, CHLE, PROS) | 0.933 |
| 27 | (CO3, APOA4, PROS) | 0.933 |
| 28 | (CO3, A1BG, PROS) | 0.933 |
| 29 | (FA9, CO3, PROS) | 0.933 |
| 30 | (CO3, C1QB, PROS) | 0.933 |
| 31 | (CO3, C1QC, PROS) | 0.933 |
| 32 | (CO3, KLKB1, PROS) | 0.933 |
| 33 | (THRB, CO3, PROS) | 0.933 |
| 34 | (CO3, C1QA, ECM1) | 0.933 |
| 35 | (HEP2, PROS, A1AG2) | 0.933 |
| 36 | (HEP2, CHLE, C4BPB) | 0.933 |
| 37 | (CO3, TTHY, PROS) | 0.933 |
| 38 | (CO3, PROS, PZP) | 0.933 |
| 39 | (CO3, C1QB, C163A) | 0.933 |
| 40 | (CO3, C1QA, KLKB1) | 0.933 |
| 41 | (CO3, HEP2, PROS) | 0.933 |
| 42 | (PROS, PHLD, ECM1) | 0.933 |
| 43 | (HEP2, VCAM1, C4BPB) | 0.933 |
| 44 | (PLF4, HEP2, APOA4) | 0.933 |
| 45 | (HEP2, A1AG2, FCGBP) | 0.933 |
| 46 | (CO3, C1QA, ITIH1) | 0.933 |
| 47 | (CO3, C1QA, LUM) | 0.933 |
| 48 | (B3AT, HEP2, PZP) | 0.933 |
| 49 | (A1AG2, C4BPB, PHLD) | 0.925 |
| 50 | (CO3, PROS, K22E) | 0.925 |
| 51 | (CO3, PROS, APOA) | 0.925 |
| 52 | (CO3, PROS, STOM) | 0.925 |
| 53 | (CERU, C4BPB, PHLD) | 0.925 |
| 54 | (C1QA, A1AG2, PHLD) | 0.925 |
| 55 | (CO3, PROS, TENA) | 0.925 |
| 56 | (AQR, CO3, C1QB) | 0.925 |
| 57 | (CO3, CO9, PROS) | 0.925 |
| 58 | (CO3, PROS, AFAM) | 0.925 |
| 59 | (B3AT, C1QA, HABP2) | 0.925 |
| 60 | (CO3, PROS, PROP) | 0.925 |
| 61 | (CO3, PROS, PRG2) | 0.925 |
| 62 | (B3AT, HEP2, PHLD) | 0.925 |
| 63 | (HEP2, C4BPB, PHLD) | 0.925 |
| 64 | (CO3, C4BPB, PHLD) | 0.925 |
| 65 | (B3AT, HEP2, APOA4) | 0.925 |
| 66 | (CERU, CO3, PROS) | 0.925 |
| 67 | (CO3, PROS, C163A) | 0.925 |
| 68 | (CO3, C1QA, C163A) | 0.924 |
| 69 | (THRB, HEP2, C4BPB) | 0.924 |
| 70 | (PLF4, PROS, A1AG2) | 0.924 |
| 71 | (CO3, C1QA, PROP) | 0.924 |
| 72 | (CO3, C1QB, PZP) | 0.924 |
| 73 | (APOL1, CO3, C1QB) | 0.924 |
| 74 | (HEP2, PROS, PHLD) | 0.924 |
| 75 | (CO3, C1QA, TENA) | 0.924 |
| 76 | (HEP2, PROS, ECM1) | 0.924 |
| 77 | (CO3, C1QA, VCAM1) | 0.924 |
| 78 | (CO3, C1QA, A1BG) | 0.924 |
| 79 | (C1QB, HEP2, C163A) | 0.924 |
| 80 | (CO3, C1QA, HGFA) | 0.924 |
| 81 | (CO3, C1QA, HABP2) | 0.924 |
| 82 | (CO3, C1QA, AT2A3) | 0.924 |
| 83 | (CO3, C1QA, C1QB) | 0.924 |
| 84 | (CO3, C1QA, STOM) | 0.924 |
| 85 | (CO3, VCAM1, C4BPB) | 0.924 |
| 86 | (CO3, C1QB, HABP2) | 0.924 |
| 87 | (C1QB, HEP2, APOA4) | 0.924 |
| 88 | (CO3, C1QB, MYH9) | 0.924 |
| 89 | (CO3, C1QB, HGFA) | 0.924 |
| 90 | (FIBA, C1QB, ECM1) | 0.924 |
| 91 | (CO3, C1QA, APOA) | 0.924 |
| 92 | (CO3, FIBA, C1QB) | 0.924 |
| 93 | (KLKB1, HEP2, PROS) | 0.924 |
| 94 | (HEP2, PROS, TSP1) | 0.924 |
| 95 | (PROS, A1AG2, C4BPB) | 0.924 |
| 96 | (CO3, PROS, FCGBP) | 0.924 |
| 97 | (CO3, PROS, LTBP1) | 0.924 |
| 98 | (PROS, A1AG2, PZP) | 0.924 |
| 99 | (A1AG2, C4BPB, HABP2) | 0.917 |
| 100 | (PROS, VCAM1, A1AG2) | 0.916 |
| 101 | (CO3, PROS, ITIH1) | 0.916 |
| 102 | (CO3, PROS, PXDC2) | 0.916 |
| 103 | (APOL1, C4BPB, PHLD) | 0.916 |
| 104 | (CO3, ALBU, PROS) | 0.916 |
| 105 | (CO3, THBG, PROS) | 0.916 |
| 106 | (APOA2, PROS, CD14) | 0.916 |
| 107 | (PROS, A1AG2, PHLD) | 0.916 |
| 108 | (APOA2, PROS, TSP1) | 0.916 |
| 109 | (CERU, PHLD, FCGBP) | 0.916 |
| 110 | (HEP2, PROS, FCGBP) | 0.916 |
| 111 | (CO3, B3AT, PROS) | 0.916 |
| 112 | (CO3, PROS, LUM) | 0.916 |
| 113 | (CO3, PROS, A2AP) | 0.916 |
| 114 | (CO3, B3AT, C1QB) | 0.916 |
| 115 | (ALBU, PROS, PHLD) | 0.916 |
| 116 | (HEP2, VCAM1, FCGBP) | 0.916 |
| 117 | (AQR, PROS, HABP2) | 0.916 |
| 118 | (CERU, CO3, C4BPB) | 0.916 |
| 119 | (CO3, C1QA, ALBU) | 0.916 |
| 120 | (CO3, B3AT, HEP2) | 0.916 |
| 121 | (TTHY, HEP2, PROS) | 0.916 |
| 122 | (FA10, CO3, C1QA) | 0.916 |
| 123 | (PLF4, HEP2, PROS) | 0.916 |
| 124 | (B3AT, HEP2, PROS) | 0.916 |
| 125 | (HEP2, C4BPB, PXDC2) | 0.916 |
| 126 | (APOA4, ITA2B, A1AG2) | 0.916 |
| 127 | (CO3, PROS, OAF) | 0.916 |
| 128 | (CO3, PROS, C4BPB) | 0.916 |
| 129 | (B3AT, HEP2, C4BPB) | 0.916 |
| 130 | (CO3, C1QA, C4BPB) | 0.916 |
| 131 | (HEP2, PROS, K22E) | 0.916 |
| 132 | (HEP2, PROS, LUM) | 0.916 |
| 133 | (HEP2, PROS, PROP) | 0.916 |
| 134 | (HEP2, PROS, MYH9) | 0.916 |
| 135 | (CO3, C1QA, AFAM) | 0.916 |
| 136 | (APOA2, C1QA, HEP2) | 0.916 |
| 137 | (AQR, A1AG2, C4BPB) | 0.916 |
| 138 | (CO3, C1QA, C1QC) | 0.916 |
| 139 | (CO3, C1QB, STOM) | 0.916 |
| 140 | (C1QB, HEP2, PHLD) | 0.916 |
| 141 | (CO3, C1QA, A2AP) | 0.916 |
| 142 | (CO3, C1QB, A1BG) | 0.916 |
| 143 | (CO3, C1QA, K22E) | 0.916 |
| 144 | (CO3, C1QA, CHLE) | 0.916 |
| 145 | (B3AT, HEP2, APOA) | 0.916 |
| 146 | (HEP2, APOA4, C4BPB) | 0.916 |
| 147 | (CO3, C1QB, THBG) | 0.916 |
| 148 | (CO3, C1QA, MYH9) | 0.916 |
| 149 | (CO3, C1QB, ITIH1) | 0.916 |
| 150 | (AQR, HEP2, PROS) | 0.916 |
| 151 | (FA9, CO3, C1QA) | 0.916 |
| 152 | (FA10, B3AT, HEP2) | 0.916 |
| 153 | (CO3, C1QB, ALBU) | 0.916 |
| 154 | (CO3, C1QA, FCGBP) | 0.916 |
| 155 | (C1QB, AMBP, HEP2) | 0.916 |
| 156 | (CO3, B3AT, FCGBP) | 0.916 |
| 157 | (VCAM1, A1AG2, FCGBP) | 0.916 |
| 158 | (B3AT, HEP2, OAF) | 0.916 |
| 159 | (CO3, C1QB, CO8B) | 0.916 |
| 160 | (CO3, C1QB, KLKB1) | 0.916 |
| 161 | (THRB, CO3, C1QB) | 0.916 |
| 162 | (AMBP, PROS, A1AG2) | 0.908 |
| 163 | (PROS, PZP, HABP2) | 0.908 |
| 164 | (FIBA, A1AG2, PHLD) | 0.908 |
| 165 | (APOA2, HEP2, FCGBP) | 0.908 |
| 166 | (CO3, PROS, AT2A3) | 0.908 |
| 167 | (B3AT, PROS, A1AG2) | 0.908 |
| 168 | (CERU, APOA2, FCGBP) | 0.908 |
| 169 | (CO3, C4BPB, HABP2) | 0.908 |
| 170 | (APOL1, CO3, PROS) | 0.908 |
| 171 | (APOA2, PHLD, FCGBP) | 0.908 |
| 172 | (APOA2, PROS, A1AG2) | 0.908 |
| 173 | (FA9, PROS, A1AG2) | 0.908 |
| 174 | (CO3, AMBP, PROS) | 0.908 |
| 175 | (FIBA, PHLD, ECM1) | 0.908 |
| 176 | (B3AT, THBG, HABP2) | 0.908 |
| 177 | (B3AT, HEP2, CHLE) | 0.908 |
| 178 | (A1AG2, PHLD, FCGBP) | 0.908 |
| 179 | (CO3, PROS, HGFA) | 0.908 |
| 180 | (APOA2, PLF4, A1AG2) | 0.908 |
| 181 | (B3AT, LUM, HABP2) | 0.908 |
| 182 | (VCAM1, C4BPB, PHLD) | 0.908 |
| 183 | (CO3, C1QA, CD14) | 0.908 |
| 184 | (CO3, C1QB, K22E) | 0.908 |
| 185 | (CO3, C1QA, OAF) | 0.908 |
| 186 | (C1QA, A1AG2, HABP2) | 0.908 |
| 187 | (APOA2, B3AT, OAF) | 0.908 |
| 188 | (CO3, C1QB, TTHY) | 0.908 |
| 189 | (B3AT, HEP2, ECM1) | 0.908 |
| 190 | (HEP2, PROS, VCAM1) | 0.908 |
| 191 | (CD14, C4BPB, PHLD) | 0.908 |
| 192 | (CERU, CO3, C1QA) | 0.908 |
| 193 | (AQR, HEP2, C4BPB) | 0.908 |
| 194 | (HEP2, PROS, PXDC2) | 0.908 |
| 195 | (APOA4, ITA2B, PHLD) | 0.908 |
| 196 | (CO3, C1QA, F13B) | 0.908 |
| 197 | (C4BPB, PHLD, ECM1) | 0.908 |
| 198 | (HEP2, PROS, ITA2B) | 0.908 |
| 199 | (THBG, PROS, HABP2) | 0.908 |
| 200 | (APOA2, PLF4, PROP) | 0.908 |
| 201 | (APOA2, PLF4, MMRN1) | 0.908 |
| 202 | (HEP2, PROS, HABP2) | 0.908 |
| 203 | (CO3, C1QA, CO8B) | 0.908 |
| 204 | (HEP2, PROS, LG3BP) | 0.908 |
| 205 | (HEP2, LUM, FCGBP) | 0.908 |
| 206 | (CO3, PROS, MMRN1) | 0.908 |
| 207 | (CO3, C1QB, CO9) | 0.908 |
| 208 | (PROS, A1AG2, OAF) | 0.908 |
| 209 | (HEP2, AFAM, FCGBP) | 0.908 |
| 210 | (PROS, TSP1, A1AG2) | 0.908 |
| 211 | (HEP2, C4BPB, LUM) | 0.908 |
| 212 | (CO3, B3AT, LTBP1) | 0.908 |
| 213 | (HEP2, CHLE, PROS) | 0.908 |
| 214 | (ANT3, CO3, C1QA) | 0.908 |
| 215 | (CO3, C1QA, PXDC2) | 0.908 |
| 216 | (HEP2, PROS, CD14) | 0.908 |
| 217 | (HEP2, C4BPB, PROP) | 0.908 |
| 218 | (PROS, CO8B, HABP2) | 0.908 |
| 219 | (KNG1, A1AG2, FCGBP) | 0.908 |
| 220 | (PROS, A1AG2, LTBP1) | 0.908 |
| 221 | (KNG1, HEP2, PROS) | 0.908 |
| 222 | (CO3, C1QA, THBG) | 0.907 |
| 223 | (CO3, C1QB, LG3BP) | 0.907 |
| 224 | (B3AT, HEP2, TENA) | 0.907 |
| 225 | (A1AG2, AFAM, FCGBP) | 0.907 |
| 226 | (C1QA, HEP2, C4BPB) | 0.907 |
| 227 | (CO3, C1QB, PRG2) | 0.907 |
| 228 | (ANT3, CO3, C1QB) | 0.907 |
| 229 | (HEP2, PROS, MMRN1) | 0.907 |
| 230 | (CO3, C1QB, C1QC) | 0.907 |
| 231 | (CO3, C1QB, LUM) | 0.907 |
| 232 | (CO3, B3AT, PZP) | 0.907 |
| 233 | (CO3, C1QB, CHLE) | 0.907 |
| 234 | (CO3, C1QB, VCAM1) | 0.907 |
| 235 | (CO3, C1QB, OAF) | 0.907 |
| 236 | (CO3, C1QB, A2AP) | 0.907 |
| 237 | (CO3, C1QB, APOA) | 0.907 |
| 238 | (B3AT, F13B, HEP2) | 0.907 |
| 239 | (HEP2, PROS, PZP) | 0.907 |
| 240 | (B3AT, HEP2, K22E) | 0.907 |
| 241 | (B3AT, HEP2, ITIH1) | 0.907 |
| 242 | (CO3, C1QB, AFAM) | 0.907 |
| 243 | (APOA2, C1QB, HEP2) | 0.907 |
| 244 | (CO3, KNG1, C1QB) | 0.907 |
| 245 | (CERU, CO3, C1QB) | 0.907 |
| 246 | (C1QB, HEP2, CHLE) | 0.907 |
| 247 | (CO3, C1QB, PXDC2) | 0.907 |
| 248 | (B3AT, TTHY, HEP2) | 0.907 |
| 249 | (CO3, C1QB, PROP) | 0.907 |
| 250 | (FIBB, HEP2, C4BPB) | 0.907 |
| 251 | (CO3, C1QA, HEP2) | 0.907 |
| 252 | (C1QA, HEP2, C163A) | 0.907 |
| 253 | (C1QA, HEP2, CHLE) | 0.907 |
| 254 | (C1QA, HEP2, ECM1) | 0.907 |
| 255 | (CO3, C1QA, PZP) | 0.907 |
| 256 | (C1QB, CXCL7, HEP2) | 0.907 |
| 257 | (THRB, CO3, C1QA) | 0.907 |

It was found that a number of cancer biomarkers were unexpectedly overrepresented in the 3plexes. Some of the overrepresented markers include C03 (individual pan cancer AUC of 0.806) that was included in 141 out of the top 257 3plexes, PROS (individual pan cancer AUC of 0.876) that was included in 101 out of the top 257 3plexes, and HEP2 (individual pan cancer AUC of 0.847) that was included in 70 out of the top 257 3plexes. It is noted that these overrepresented biomarkers are not necessarily the best performing individual pan cancer biomarkers, based on AUC score (see Table 8.2). Among the pan cancer 3plexes listed in Table 83, the 20 most frequently identified proteins from the 3plexes are listed in Table 8.4.

TABLE 8.4
Most common proteins in pan cancer 3plexes

| # | protein | 3plex count |
|---|---|---|
| 1 | CO3 | 141 |
| 2 | PROS | 101 |
| 3 | HEP2 | 70 |
| 4 | C1QA | 48 |
| 5 | C1QB | 47 |
| 6 | C4BPB | 29 |
| 7 | A1AG2 | 28 |
| 8 | B3AT | 27 |
| 9 | PHLD | 22 |
| 10 | FCGBP | 16 |
| 11 | HABP2 | 14 |
| 12 | APOA2 | 13 |
| 13 | VCAM1 | 10 |
| 14 | ECM1 | 9 |
| 15 | PZP | 8 |
| 16 | CHLE | 8 |
| 17 | APOA4 | 8 |
| 18 | LUM | 7 |
| 19 | CERU | 7 |
| 20 | PROP | 6 |

The top ranked pan cancer 3plexes (those listed in Table 8.3) were selected to generate Logistic regression equations as classifiers for future prediction of samples of unknown cancer/non-cancer status. Logistic regression using Scikit-learn package with ‘newton-cg’ solver and no penalty was used to create a cancer prediction equation as follows:

$$Probability ( target = ‘ Cancer ’ ) = 1 / 1 + exp \hat{} - ( beta 0 + beta 1 * Protein 1 + beta 2 * Protein 2 + beta 3 * Protein 3 )$$

where probability >0.5 is cancer and probability <=0.5 is normal (non-cancer)
where beta 0 is an intercept or bias coefficient, beta 1 is a beta coefficient for Protein 1, beta2 is a beta coefficient for Protein 2, and beta3 is a beta coefficient for Protein3.
Protein1, Protein2 and Protein3 are the quantitative measures, respectively of each biomarker.

The betas (e.g., beta1, beta2, beta3) represent a beta coefficient for each of the proteins. The respective values of the betas for each of the biomarkers were empirically learned through a machine learning training process, and a given beta describes the size and direction of the relationship between a quantitative measure of a given biomarker and the outcome variable (e.g., “Probability” in the equation above). For example, changing the measured value of a given biomarker (e.g. Protein2) by 1 unit changes the value of outcome variable by the value of the corresponding beta coefficient (e.g. beta2) when all other proteins remain fixed. The first coefficient in the sum, beta0, is an intercept coefficient or bias. The intercept coefficient reflects the predicted outcome of an instance where all proteins are at their mean value.

The short listed 3plexes (or a larger multiplex comprising one or more of the 3plexes, and optionally other biomarkers) may thus be used for predicting presence or likelihood of cancer of subject based on quantification of the given combination of proteins.

### Example 9—Novel Machine Learning Tools Applied to Individual Cancer Indications

Following the methods outlined above for pan cancer, additional analysis was performed on each of the four individual cancer indication subsets. This process led to the identification of scores of significantly proteins used for the identification of scores of differentially expressed proteins in each indication, as shown in Table 9.1. In table 9.1 The “Number of samples” column represents the total number of individual patient samples (including 25 non-cancer subjects) for each indication. The “Valid intensity proteins” column represents the number of detected proteins that passed an initial validation. The “Significant proteins” column represent proteins with a q-value<0.05, and the “Predictive 3plexes” column represents the total number of 3plexes identified from the stated “Significant proteins” where the accuracy cutoff for each indication was either 90% (pan cancer, breast cancer, lung cancer) or 95% (ovarian cancer and colorectal cancer).

TABLE 9.1
Summary of 3plex generation

| | Number | Valid | | | Plex |
|---|---|---|---|---|---|
| | of | intensity | Significant | Predictive | Accuracy |
| Indication | samples | proteins | proteins | 3plexes | cutoff |
| Pan Cancer | 119 | 235 | 61 | 257 | 0.9 |
| Ovarian Cancer | 50 | 231 | 58 | 141 | 0.95 |
| Breast Cancer | 50 | 230 | 36 | 63 | 0.9 |
| Lung Cancer | 44 | 241 | 29 | 135 | 0.9 |
| CRC | 50 | 237 | 53 | 272 | 0.95 |

### Ovarian Cancer Biomarkers and 3Plexes

Following the methods outlined above for pan cancer in Example 8, the ovarian cancer cohort was similarly assessed separately, to identify a list of ovarian cancer biomarkers, as well as generate a list of predictive 3plexes of ovarian cancer biomarkers that demonstrated a high accuracy score. Table 9.2 lists the 58 ovarian cancer biomarkers from the ovarian cancer dataset that demonstrated highly statistically significant differential expression compared to the non-cancer cohort based on p-value, q-value, log 2FC, and AUC score. Each row represents one of the 58 ovarian cancer biomarkers, and is identified by a respective UniprotKB unique protein entry name (column 1; “Protein”), UniprotKB unique accession number (column 2; “Uniprot AN”), and colloquial protein name (column 3; “Protein Name”). Note that the “_HUMAN” suffix was omitted from each of the protein entry names in column 1, for clarity of presentation. Column 4 shows a p-value denoting statistical significance. Column 5 shows a q-value, which is an adjusted p-value using Benjamini-Hochberg correction. Column 6 shows log 2FC, indicating the scale and direction of differential expression in Log2 units, where a negative value indicates downregulation in the cancer cohort compared to the non-cancer cohort and a positive value indicates upregulation in the cancer cohort compared to the non-cancer cohort. Column 7 shows the area under the curve (AUC) of the ROC curve generated from the quantification data for each protein.

TABLE 9.2
Significantly differentially expressed ovarian cancer biomarkers

| Protein | Uniprot AC | Protein Name | p-value | q-value | log2FC | AUC |
|---|---|---|---|---|---|---|
| APOL1 | O14791 | Apolipoprotein L1 | 3.45E−04 | 3.98E−03 | −0.583 | 0.752 |
| AQR | O60306 | RNA helicase aquarius | 1.06E−03 | 8.12E−03 | 5.963 | 0.796 |
| ATRN | O75882 | Attractin | 6.24E−03 | 2.83E−02 | −1.911 | 0.856 |
| CERU | P00450 | Ceruloplasmin | 2.79E−04 | 3.64E−03 | −0.589 | 0.736 |
| F13A | P00488 | Coagulation factor XIII A chain | 4.30E−03 | 2.26E−02 | −0.736 | 0.712 |
| FA10 | P00742 | Coagulation factor X | 2.09E−04 | 3.04E−03 | −0.800 | 0.784 |
| A2MG | P01023 | Alpha-2-macroglobulin | 2.10E−04 | 3.04E−03 | −0.560 | 0.776 |
| KNG1 | P01042 | Kininogen-1 | 4.68E−03 | 2.30E−02 | −0.664 | 0.792 |
| PIGR | P01833 | Polymeric immunoglobulin | 3.07E−03 | 1.68E−02 | −2.594 | 0.864 |
| | | receptor | | | | |
| APOE | P02649 | Apolipoprotein E | 3.05E−03 | 1.68E−02 | 0.519 | 0.720 |
| APOA2 | P02652 | Apolipoprotein A-II | 2.63E−03 | 1.64E−02 | −0.855 | 0.784 |
| FIBA | P02671 | Fibrinogen alpha chain | 1.58E−05 | 5.22E−04 | 0.773 | 0.824 |
| FIBB | P02675 | Fibrinogen beta chain | 5.01E−03 | 2.41E−02 | 0.820 | 0.744 |
| B3AT | P02730 | Band 3 anion transport protein | 1.65E−04 | 2.94E−03 | 5.044 | 0.896 |
| C1QA | P02745 | Complement C1q subcomponent | 7.42E−05 | 1.74E−03 | 1.439 | 0.864 |
| | | subunit A | | | | |
| C1QB | P02746 | Complement C1q subcomponent | 4.45E−03 | 2.28E−02 | 2.351 | 0.896 |
| | | subunit B | | | | |
| C1QC | P02747 | Complement C1q subcomponent | 7.32E−05 | 1.74E−03 | 2.549 | 0.776 |
| | | subunit C | | | | |
| A2GL | P02750 | Leucine-rich alpha-2- | 2.81E−03 | 1.68E−02 | 0.647 | 0.672 |
| | | glycoprotein | | | | |
| AMBP | P02760 | Protein AMBP | 1.05E−04 | 2.02E−03 | −0.792 | 0.832 |
| TTHY | P02766 | Transthyretin | 3.12E−03 | 1.68E−02 | −1.045 | 0.816 |
| PLF4 | P02776 | Platelet factor 4 | 9.57E−04 | 7.62E−03 | 3.739 | 0.792 |
| KLKB1 | P03952 | Plasma kallikrein | 2.96E−06 | 1.69E−04 | −0.865 | 0.864 |
| CATA | P04040 | Catalase | 1.09E−03 | 8.12E−03 | 4.921 | 0.800 |
| F13B | P05160 | Coagulation factor XIII B chain | 6.06E−04 | 5.83E−03 | −0.762 | 0.808 |
| THBG | P05543 | Thyroxine-binding globulin | 4.96E−04 | 4.98E−03 | −5.749 | 0.784 |
| HEP2 | P05546 | Heparin cofactor 2 | 2.96E−04 | 3.64E−03 | −1.052 | 0.864 |
| CHLE | P06276 | Cholinesterase | 1.21E−06 | 1.40E−04 | −0.935 | 0.848 |
| GELS | P06396 | Gelsolin | 2.41E−03 | 1.55E−02 | −0.670 | 0.720 |
| APOA4 | P06727 | Apolipoprotein A-IV | 2.25E−07 | 5.21E−05 | −2.065 | 0.944 |
| PROS | P07225 | Vitamin K-dependent protein S | 8.11E−04 | 6.69E−03 | 0.947 | 0.912 |
| CO8B | P07358 | Complement component C8 beta | 1.24E−02 | 4.93E−02 | −0.466 | 0.664 |
| | | chain | | | | |
| TSP1 | P07996 | Thrombospondin-1 | 7.43E−04 | 6.69E−03 | 5.743 | 0.760 |
| APOA | P08519 | Apolipoprotein(a) | 1.18E−03 | 8.52E−03 | 1.986 | 0.736 |
| A2AP | P08697 | Alpha-2-antiplasmin | 3.04E−03 | 1.68E−02 | −0.437 | 0.712 |
| IGA2 | P0DOX2 | Immunoglobulin alpha-2 heavy | 1.19E−02 | 4.83E−02 | 4.510 | 0.728 |
| | | chain | | | | |
| VCAM1 | P19320 | Vascular cell adhesion protein 1 | 3.12E−06 | 1.69E−04 | −1.105 | 0.832 |
| PZP | P20742 | Pregnancy zone protein | 4.66E−03 | 2.30E−02 | 6.396 | 0.636 |
| C4BPB | P20851 | C4b-binding protein beta chain | 9.90E−06 | 3.81E−04 | 1.263 | 0.840 |
| STOM | P27105 | Stomatin | 7.72E−04 | 6.69E−03 | 4.480 | 0.748 |
| BTD | P43251 | Biotinidase | 2.99E−04 | 3.64E−03 | −1.076 | 0.840 |
| AFAM | P43652 | Afamin | 1.90E−04 | 3.04E−03 | −0.958 | 0.784 |
| NOTC1 | P46531 | Neurogenic locus notch homolog | 3.96E−04 | 4.35E−03 | −5.950 | 0.872 |
| | | protein 1 | | | | |
| COMP | P49747 | Cartilage oligomeric matrix | 1.48E−03 | 9.74E−03 | −0.890 | 0.768 |
| | | protein | | | | |
| HBA | P69905 | Hemoglobin subunit alpha | 7.71E−03 | 3.36E−02 | 0.646 | 0.720 |
| PHLD | P80108 | Phosphatidylinositol-glycan- | 7.97E−04 | 6.69E−03 | −3.128 | 0.944 |
| | | specific phospholipase D | | | | |
| HGFA | Q04756 | Hepatocyte growth factor | 5.31E−03 | 2.45E−02 | −1.011 | 0.672 |
| | | activator | | | | |
| LRP1 | Q07954 | Prolow-density lipoprotein | 8.06E−03 | 3.45E−02 | −3.874 | 0.704 |
| | | receptor-related protein 1 | | | | |
| MMRN1 | Q13201 | Multimerin-1 | 1.15E−02 | 4.76E−02 | 4.611 | 0.688 |
| SPRL1 | Q14515 | SPARC-like protein 1 | 9.99E−03 | 4.20E−02 | −4.177 | 0.692 |
| HABP2 | Q14520 | Hyaluronan-binding protein 2 | 3.65E−06 | 1.69E−04 | −2.394 | 0.896 |
| ECM1 | Q16610 | Extracellular matrix protein 1 | 7.53E−05 | 1.74E−03 | −0.873 | 0.816 |
| PXDC2 | Q6UX71 | Plexin domain-containing protein | 8.62E−05 | 1.81E−03 | −0.505 | 0.840 |
| | | 2 | | | | |
| OAF | Q86UD1 | Out at first protein homolog | 5.26E−03 | 2.45E−02 | −4.367 | 0.700 |
| C163A | Q86VB7 | Scavenger receptor cysteine-rich | 1.38E−03 | 9.52E−03 | −5.523 | 0.848 |
| | | type 1 protein M130 | | | | |
| ZN483 | Q8TF39 | Zinc finger protein 483 | 6.57E−03 | 2.92E−02 | 1.267 | 0.760 |
| PCYOX | Q9UHG3 | Prenylcysteine oxidase 1 | 1.40E−03 | 9.52E−03 | −0.994 | 0.688 |
| HEG1 | Q9ULI3 | Protein HEG homolog 1 | 4.71E−04 | 4.95E−03 | −0.792 | 0.784 |
| FCGBP | Q9Y6R7 | IgGFc-binding protein | 2.83E−03 | 1.68E−02 | 0.842 | 0.736 |

Table 9.3 lists the 141 top-performing 3plexes generated from the ovarian cancer biomarkers provided in Table 9.2. Each 3plex listed in Table 9.3 achieved an accuracy score (i.e., a 5-fold average accuracy score calculated as describe above in Example 8) of 0.95 (95%) or higher, representing a correct-prediction ratio of 0.95 or higher.

TABLE 9.3
Ovarian Cancer 3plexes with Accuracy >0.95

| | | 5-Fold average |
|---|---|---|
| # | 3PLEX | Accuracy |
| 1 | (NOTC1, PHLD, FCGBP) | 1 |
| 2 | (VCAM1, HEG1, FCGBP) | 0.98 |
| 3 | (PIGR, F13B, PROS) | 0.98 |
| 4 | (C1QA, C4BPB, HABP2) | 0.98 |
| 5 | (C4BPB, HABP2, ZN483) | 0.98 |
| 6 | (APOE, C4BPB, PHLD) | 0.98 |
| 7 | (APOE, C4BPB, HABP2) | 0.98 |
| 8 | (FIBA, PHLD, FCGBP) | 0.98 |
| 9 | (APOL1, CERU, C4BPB) | 0.98 |
| 10 | (FIBA, CHLE, APOA4) | 0.98 |
| 11 | (FA10, FIBA, APOA4) | 0.98 |
| 12 | (APOL1, APOA4, C4BPB) | 0.98 |
| 13 | (BTD, PHLD, FCGBP) | 0.98 |
| 14 | (APOL1, APOA4, FCGBP) | 0.98 |
| 15 | (APOA2, VCAM1, FCGBP) | 0.98 |
| 16 | (F13A, FIBA, C1QA) | 0.98 |
| 17 | (KLKB1, APOA4, C4BPB) | 0.98 |
| 18 | (F13A, PIGR, C1QB) | 0.98 |
| 19 | (AMBP, VCAM1, FCGBP) | 0.98 |
| 20 | (CERU, APOA4, C4BPB) | 0.98 |
| 21 | (PHLD, HEG1, FCGBP) | 0.98 |
| 22 | (APOL1, C1QB, C4BPB) | 0.96 |
| 23 | (AMBP, CHLE, C4BPB) | 0.96 |
| 24 | (APOL1, C1QC, C4BPB) | 0.96 |
| 25 | (F13A, B3AT, HEP2) | 0.96 |
| 26 | (STOM, PHLD, FCGBP) | 0.96 |
| 27 | (A2GL, APOA4, PXDC2) | 0.96 |
| 28 | (THBG, C4BPB, HABP2) | 0.96 |
| 29 | (KLKB1, C4BPB, HABP2) | 0.96 |
| 30 | (HEP2, VCAM1, C4BPB) | 0.96 |
| 31 | (APOA4, A2AP, NOTC1) | 0.96 |
| 32 | (FIBA, APOA4, CO8B) | 0.96 |
| 33 | (F13A, APOA4, FCGBP) | 0.96 |
| 34 | (F13A, FIBA, B3AT) | 0.96 |
| 35 | (ATRN, PHLD, FCGBP) | 0.96 |
| 36 | (AFAM, PHLD, FCGBP) | 0.96 |
| 37 | (PIGR, FIBA, ECM1) | 0.96 |
| 38 | (FIBA, F13B, APOA4) | 0.96 |
| 39 | (APOA4, IGA2, VCAM1) | 0.96 |
| 40 | (ATRN, C4BPB, HABP2) | 0.96 |
| 41 | (FIBB, APOA4, APOA) | 0.96 |
| 42 | (FIBA, FIBB, APOA4) | 0.96 |
| 43 | (THBG, PHLD, FCGBP) | 0.96 |
| 44 | (THBG, APOA4, PROS) | 0.96 |
| 45 | (FIBA, AMBP, APOA4) | 0.96 |
| 46 | (A2AP, C4BPB, HABP2) | 0.96 |
| 47 | (VCAM1, ECM1, FCGBP) | 0.96 |
| 48 | (KNG1, APOA4, C4BPB) | 0.96 |
| 49 | (FIBA, C1QB, APOA4) | 0.96 |
| 50 | (APOE, CHLE, C4BPB) | 0.96 |
| 51 | (B3AT, CHLE, APOA4) | 0.96 |
| 52 | (F13A, APOA4, APOA) | 0.96 |
| 53 | (C4BPB, HABP2, FCGBP) | 0.96 |
| 54 | (FIBA, APOA4, VCAM1) | 0.96 |
| 55 | (C4BPB, NOTC1, HABP2) | 0.96 |
| 56 | (A2GL, PROS, PHLD) | 0.96 |
| 57 | (APOL1, APOE, C4BPB) | 0.96 |
| 58 | (F13A, FIBA, APOA4) | 0.96 |
| 59 | (B3AT, C4BPB, PHLD) | 0.96 |
| 60 | (A2GL, C4BPB, HABP2) | 0.96 |
| 61 | (F13B, PHLD, FCGBP) | 0.96 |
| 62 | (FIBA, APOA4, HEG1) | 0.96 |
| 63 | (AQR, C4BPB, HABP2) | 0.96 |
| 64 | (APOL1, F13B, C4BPB) | 0.96 |
| 65 | (C4BPB, PHLD, LRP1) | 0.96 |
| 66 | (F13A, FIBA, PZP) | 0.96 |
| 67 | (C4BPB, AFAM, HABP2) | 0.96 |
| 68 | (FIBA, C4BPB, HABP2) | 0.96 |
| 69 | (APOL1, A2AP, C4BPB) | 0.96 |
| 70 | (FIBA, VCAM1, C4BPB) | 0.96 |
| 71 | (F13B, C4BPB, HABP2) | 0.96 |
| 72 | (C4BPB, BTD, HABP2) | 0.96 |
| 73 | (FA10, C4BPB, HABP2) | 0.96 |
| 74 | (C4BPB, HBA, HABP2) | 0.96 |
| 75 | (FIBA, APOA4, PXDC2) | 0.96 |
| 76 | (APOA4, AFAM, FCGBP) | 0.96 |
| 77 | (PZP, PHLD, FCGBP) | 0.96 |
| 78 | (KNG1, PHLD, FCGBP) | 0.96 |
| 79 | (C1QB, PHLD, FCGBP) | 0.96 |
| 80 | (C1QB, APOA4, HBA) | 0.96 |
| 81 | (A2GL, PHLD, FCGBP) | 0.96 |
| 82 | (C4BPB, HABP2, OAF) | 0.96 |
| 83 | (C4BPB, HABP2, ECM1) | 0.96 |
| 84 | (FIBA, APOA4, NOTC1) | 0.96 |
| 85 | (FIBA, APOA4, COMP) | 0.96 |
| 86 | (C4BPB, HGFA, HABP2) | 0.96 |
| 87 | (FIBA, APOA4, ECM1) | 0.96 |
| 88 | (FIBA, APOA4, PHLD) | 0.96 |
| 89 | (FIBA, APOA4, HGFA) | 0.96 |
| 90 | (FIBA, APOA4, MMRN1) | 0.96 |
| 91 | (A2MG, C1QB, APOA4) | 0.96 |
| 92 | (C4BPB, PHLD, OAF) | 0.96 |
| 93 | (FIBA, APOA4, HABP2) | 0.96 |
| 94 | (C4BPB, PHLD, ECM1) | 0.96 |
| 95 | (TSP1, C4BPB, HABP2) | 0.96 |
| 96 | (F13A, C1QB, SPRL1) | 0.96 |
| 97 | (KNG1, C4BPB, HABP2) | 0.96 |
| 98 | (APOA2, PHLD, FCGBP) | 0.96 |
| 99 | (C1QC, C4BPB, HABP2) | 0.96 |
| 100 | (FIBB, C4BPB, HABP2) | 0.96 |
| 101 | (AMBP, PHLD, FCGBP) | 0.96 |
| 102 | (CERU, PHLD, FCGBP) | 0.96 |
| 103 | (CATA, C4BPB, PHLD) | 0.96 |
| 104 | (PHLD, ECM1, FCGBP) | 0.96 |
| 105 | (APOA2, FIBA, APOA4) | 0.96 |
| 106 | (CATA, C4BPB, HABP2) | 0.96 |
| 107 | (CERU, C4BPB, PHLD) | 0.96 |
| 108 | (TTHY, THBG, APOA4) | 0.96 |
| 109 | (APOA2, APOA4, FCGBP) | 0.96 |
| 110 | (APOL1, C4BPB, PHLD) | 0.96 |
| 111 | (F13B, HEP2, PROS) | 0.96 |
| 112 | (APOE, HABP2, C163A) | 0.96 |
| 113 | (COMP, PHLD, FCGBP) | 0.96 |
| 114 | (APOA4, PHLD, FCGBP) | 0.96 |
| 115 | (APOE, B3AT, CHLE) | 0.96 |
| 116 | (CERU, C4BPB, HABP2) | 0.96 |
| 117 | (APOE, PHLD, FCGBP) | 0.96 |
| 118 | (B3AT, KLKB1, APOA4) | 0.96 |
| 119 | (APOA4, C4BPB, NOTC1) | 0.96 |
| 120 | (VCAM1, C4BPB, NOTC1) | 0.96 |
| 121 | (F13A, C1QB, C4BPB) | 0.96 |
| 122 | (C1QA, PHLD, FCGBP) | 0.96 |
| 123 | (APOA2, PLF4, ZN483) | 0.96 |
| 124 | (APOA4, C4BPB, PHLD) | 0.96 |
| 125 | (CHLE, APOA4, FCGBP) | 0.96 |
| 126 | (CERU, C4BPB, NOTC1) | 0.96 |
| 127 | (PHLD, PXDC2, FCGBP) | 0.96 |
| 128 | (APOA2, C4BPB, ZN483) | 0.96 |
| 129 | (PROS, C4BPB, HABP2) | 0.96 |
| 130 | (F13A, APOA4, C4BPB) | 0.96 |
| 131 | (PIGR, B3AT, APOA4) | 0.96 |
| 132 | (FA10, APOA4, FCGBP) | 0.96 |
| 133 | (APOA2, C1QB, FCGBP) | 0.96 |
| 134 | (C1QB, APOA4, PZP) | 0.96 |
| 135 | (C1QB, AMBP, APOA4) | 0.96 |
| 136 | (F13A, PHLD, FCGBP) | 0.96 |
| 137 | (CHLE, CO8B, C4BPB) | 0.96 |
| 138 | (PHLD, C163A, FCGBP) | 0.96 |
| 139 | (HEP2, HEG1, FCGBP) | 0.96 |
| 140 | (PHLD, ZN483, FCGBP) | 0.96 |
| 141 | (HEP2, PHLD, FCGBP) | 0.96 |

It was found that a number of cancer biomarkers were unexpectedly overrepresented in the 3plexes, and were deemed as key biomarkers for ovarian cancer. Some of the key biomarkers in ovarian cancer include C4BPB that was included in 57 out of the top 141 3plexes, APOA4 that was included in 47 out of the top 141 3plexes, FCGBP that was included in 39 out of the top 141 3plexes, and PHLD that was included in 37 out of the top 141 3plexes. Among the list of ovarian cancer 3plexes provided in Table 93, the 20 most frequently identified proteins this analysis are listed in Table 9.4.

TABLE 9.4
Most common proteins in ovarian cancer biomarker 3plexes

| | Ovarian | 3plex |
|---|---|---|
| # | Cancer | count |
| 1 | C4BPB | 57 |
| 2 | APOA4 | 47 |
| 3 | FCGBP | 39 |
| 4 | PHLD | 37 |
| 5 | HABP2 | 29 |
| 6 | FIBA | 26 |
| 7 | F13A | 12 |
| 8 | C1QB | 11 |
| 9 | VCAM1 | 9 |
| 10 | APOL1 | 9 |
| 11 | NOTC1 | 7 |
| 12 | CHLE | 7 |
| 13 | B3AT | 7 |
| 14 | APOE | 7 |
| 15 | APOA2 | 7 |
| 16 | F13B | 6 |
| 17 | ECM1 | 6 |
| 18 | CERU | 6 |
| 19 | PROS | 5 |
| 20 | HEP2 | 5 |

The top ranked ovarian 3plexes (those listed in Table 9.3) may then be selected to generate Logistic regression equations as classifiers for future prediction ofsamples of unknown cancer/non-cancer status, following the methods outlined above for pan-cancer in Example 8.

Lung CancerBiomarkers and 3plexes

Following the methods outlined above, the NSCLC cohort was separately assessed to identifly a list of lung cancer biomarkers, as well as generate a list of predictive 3plexes of the biomarkers that demonstrated a high accuracy score. Table 9.5 lists the 29 proteins from the NSCLC data set that demonstrated highly statistically significant differential expression compared to the non-cancer cohort based on p-value, q-value, log 2FC, and AUC score.

TABLE 9.5
Significantly differentially expressed lung cancer biomarkers

| Protein | Uniprot AN | Protein Name | p-value | q-value | log2FC | AUC |
|---|---|---|---|---|---|---|
| APOL1 | O14791 | Apolipoprotein L1 | 1.72E−03 | 2.66E−02 | −0.593 | 0.705 |
| AQR | O60306 | RNA helicase aquarius | 3.17E−03 | 3.64E−02 | 5.553 | 0.763 |
| CERU | P00450 | Ceruloplasmin | 5.29E−04 | 1.35E−02 | −0.690 | 0.740 |
| FIBA | P02671 | Fibrinogen alpha chain | 3.68E−03 | 3.80E−02 | 0.673 | 0.693 |
| B3AT | P02730 | Band 3 anion transport protein | 1.29E−03 | 2.39E−02 | 4.165 | 0.777 |
| C1QA | P02745 | Complement C1q | 1.79E−03 | 2.66E−02 | 1.152 | 0.800 |
| | | subcomponent subunit A | | | | |
| C1QC | P02747 | Complement C1q | 2.87E−03 | 3.46E−02 | 2.036 | 0.707 |
| | | subcomponent subunit C | | | | |
| AMBP | P02760 | Protein AMBP | 1.88E−03 | 2.66E−02 | −0.550 | 0.782 |
| PLF4 | P02776 | Platelet factor 4 | 5.60E−03 | 4.65E−02 | 3.003 | 0.710 |
| KLKB1 | P03952 | Plasma kallikrein | 5.02E−07 | 6.05E−05 | −0.876 | 0.880 |
| A1BG | P04217 | Alpha-1B-glycoprotein | 7.99E−04 | 1.61E−02 | −0.575 | 0.722 |
| THBG | P05543 | Thyroxine-binding globulin | 3.54E−03 | 3.80E−02 | −4.857 | 0.937 |
| HEP2 | P05546 | Heparin cofactor 2 | 8.00E−04 | 1.61E−02 | −1.111 | 0.903 |
| MYL1 | P05976 | Myosin light chain 1/3, skeletal | 5.06E−03 | 4.48E−02 | 4.148 | 0.767 |
| | | muscle isoform | | | | |
| CHLE | P06276 | Cholinesterase | 1.75E−05 | 1.40E−03 | −0.729 | 0.810 |
| APOA4 | P06727 | Apolipoprotein A-IV | 7.06E−05 | 4.25E−03 | −1.082 | 0.840 |
| PROS | P07225 | Vitamin K-dependent protein S | 5.20E−03 | 4.48E−02 | 0.898 | 0.897 |
| CO8B | P07358 | Complement component C8 | 5.59E−04 | 1.35E−02 | −0.760 | 0.780 |
| | | beta chain | | | | |
| CO6 | P13671 | Complement component C6 | 1.85E−03 | 2.66E−02 | 0.939 | 0.730 |
| VCAM1 | P19320 | Vascular cell adhesion protein 1 | 4.39E−04 | 1.35E−02 | −0.909 | 0.710 |
| C4BPB | P20851 | C4b-binding protein beta chain | 1.46E−04 | 7.06E−03 | 1.094 | 0.753 |
| STOM | P27105 | Stomatin | 3.95E−03 | 3.80E−02 | 3.892 | 0.757 |
| AFAM | P43652 | Afamin | 4.85E−04 | 1.35E−02 | −0.753 | 0.680 |
| LUM | P51884 | Lumican | 2.22E−03 | 2.97E−02 | −0.677 | 0.630 |
| PHLD | P80108 | Phosphatidylinositol-glycan- | 1.97E−07 | 4.75E−05 | −1.578 | 0.960 |
| | | specific phospholipase D | | | | |
| HABP2 | Q14520 | Hyaluronan-binding protein 2 | 2.20E−04 | 8.84E−03 | −1.944 | 0.830 |
| ECM1 | Q16610 | Extracellular matrix protein 1 | 2.38E−03 | 3.02E−02 | −0.606 | 0.774 |
| PXDC2 | Q6UX71 | Plexin domain-containing | 3.81E−03 | 3.80E−02 | −0.429 | 0.670 |
| | | protein 2 | | | | |
| FCGBP | Q9Y6R7 | IgGFc-binding protein | 4.51E−03 | 4.18E−02 | 0.960 | 0.730 |

Table 9.6 lists the top-performing 3plexes generated from the lung cancer biomarkers provided in Table 9.5. Each of the 135 3plexes listed in Table 9.6 achieved an accuracy score (i.e., a 5-fold average accuracy score calculated as describe above in Example 8) of 0.90 (90%) or higher, representing a correct-prediction ratio of 0.90 or higher.

TABLE 9.6
Lung Cancer 3plexes with Accuracy >0.90

| | | 5-Fold |
|---|---|---|
| | | average |
| # | 3PLEX | Accuracy |
| 1 | (CHLE, APOA4, PROS) | 0.96 |
| 2 | (APOL1, C4BPB, PHLD) | 0.96 |
| 3 | (PLF4, HEP2, MYL1) | 0.96 |
| 4 | (CERU, C4BPB, PHLD) | 0.96 |
| 5 | (CERU, HEP2, C4BPB) | 0.95 |
| 6 | (HEP2, APOA4, PROS) | 0.95 |
| 7 | (HEP2, PROS, PHLD) | 0.95 |
| 8 | (HEP2, VCAM1, FCGBP) | 0.95 |
| 9 | (APOL1, CERU, C4BPB) | 0.95 |
| 10 | (HEP2, MYL1, C4BPB) | 0.95 |
| 11 | (KLKB1, APOA4, PROS) | 0.93 |
| 12 | (FIBA, PROS, ECM1) | 0.93 |
| 13 | (APOL1, APOA4, PROS) | 0.93 |
| 14 | (AMBP, APOA4, PROS) | 0.93 |
| 15 | (KLKB1, HEP2, PROS) | 0.93 |
| 16 | (AMBP, PROS, ECM1) | 0.93 |
| 17 | (APOA4, PROS, LUM) | 0.93 |
| 18 | (B3AT, HEP2, PROS) | 0.93 |
| 19 | (APOL1, LUM, FCGBP) | 0.93 |
| 20 | (B3AT, PLF4, HEP2) | 0.93 |
| 21 | (APOA4, PROS, VCAM1) | 0.93 |
| 22 | (HEP2, PROS, ECM1) | 0.93 |
| 23 | (C1QA, PLF4, HEP2) | 0.93 |
| 24 | (THBG, C4BPB, PHLD) | 0.93 |
| 25 | (HEP2, VCAM1, C4BPB) | 0.93 |
| 26 | (HEP2, PROS, CO8B) | 0.93 |
| 27 | (HEP2, C4BPB, LUM) | 0.93 |
| 28 | (VCAM1, PHLD, FCGBP) | 0.93 |
| 29 | (PLF4, HEP2, APOA4) | 0.93 |
| 30 | (VCAM1, HABP2, FCGBP) | 0.93 |
| 31 | (APOL1, PROS, C4BPB) | 0.93 |
| 32 | (CO8B, C4BPB, HABP2) | 0.93 |
| 33 | (HEP2, MYL1, PROS) | 0.93 |
| 34 | (PLF4, HEP2, CO8B) | 0.93 |
| 35 | (HEP2, CHLE, PROS) | 0.93 |
| 36 | (CERU, C4BPB, HABP2) | 0.93 |
| 37 | (KLKB1, HEP2, C4BPB) | 0.93 |
| 38 | (KLKB1, C4BPB, HABP2) | 0.93 |
| 39 | (PLF4, KLKB1, HEP2) | 0.93 |
| 40 | (PROS, VCAM1, FCGBP) | 0.93 |
| 41 | (KLKB1, CHLE, C4BPB) | 0.93 |
| 42 | (PROS, LUM, FCGBP) | 0.93 |
| 43 | (PLF4, A1BG, HEP2) | 0.93 |
| 44 | (KLKB1, C4BPB, PXDC2) | 0.93 |
| 45 | (PLF4, THBG, HEP2) | 0.93 |
| 46 | (APOL1, VCAM1, C4BPB) | 0.93 |
| 47 | (PLF4, HEP2, CHLE) | 0.93 |
| 48 | (APOL1, HEP2, C4BPB) | 0.93 |
| 49 | (KLKB1, C4BPB, LUM) | 0.93 |
| 50 | (CERU, APOA4, PROS) | 0.91 |
| 51 | (B3AT, A1BG, HEP2) | 0.91 |
| 52 | (B3AT, HEP2, CO8B) | 0.91 |
| 53 | (FIBA, APOA4, LUM) | 0.91 |
| 54 | (CERU, FIBA, CHLE) | 0.91 |
| 55 | (APOL1, FIBA, PROS) | 0.91 |
| 56 | (CHLE, PROS, ECM1) | 0.91 |
| 57 | (B3AT, HEP2, PHLD) | 0.91 |
| 58 | (B3AT, HEP2, PXDC2) | 0.91 |
| 59 | (B3AT, HEP2, CHLE) | 0.91 |
| 60 | (FIBA, KLKB1, APOA4) | 0.91 |
| 61 | (APOA4, PROS, PHLD) | 0.91 |
| 62 | (A1BG, APOA4, PROS) | 0.91 |
| 63 | (B3AT, C1QC, HEP2) | 0.91 |
| 64 | (B3AT, STOM, PHLD) | 0.91 |
| 65 | (APOA4, PROS, ECM1) | 0.91 |
| 66 | (APOA4, PROS, AFAM) | 0.91 |
| 67 | (AMBP, HEP2, PROS) | 0.91 |
| 68 | (AMBP, KLKB1, PROS) | 0.91 |
| 69 | (APOA4, PROS, PXDC2) | 0.91 |
| 70 | (FIBA, HEP2, PROS) | 0.91 |
| 71 | (CERU, C4BPB, AFAM) | 0.91 |
| 72 | (AMBP, THBG, PROS) | 0.91 |
| 73 | (CO8B, PHLD, FCGBP) | 0.91 |
| 74 | (APOA4, PHLD, FCGBP) | 0.91 |
| 75 | (AQR, CERU, C4BPB) | 0.91 |
| 76 | (HEP2, C4BPB, HABP2) | 0.91 |
| 77 | (A1BG, HEP2, PROS) | 0.91 |
| 78 | (PLF4, HEP2, CO6) | 0.91 |
| 79 | (CERU, THBG, C4BPB) | 0.91 |
| 80 | (CO6, LUM, FCGBP) | 0.91 |
| 81 | (HEP2, PROS, FCGBP) | 0.91 |
| 82 | (HEP2, PROS, PXDC2) | 0.91 |
| 83 | (C1QC, HEP2, PROS) | 0.91 |
| 84 | (KLKB1, CO8B, FCGBP) | 0.91 |
| 85 | (HEP2, PROS, LUM) | 0.91 |
| 86 | (HEP2, PROS, STOM) | 0.91 |
| 87 | (HEP2, PROS, C4BPB) | 0.91 |
| 88 | (HEP2, PROS, VCAM1) | 0.91 |
| 89 | (HEP2, PHLD, FCGBP) | 0.91 |
| 90 | (PLF4, HEP2, C4BPB) | 0.91 |
| 91 | (KLKB1, LUM, FCGBP) | 0.91 |
| 92 | (THBG, HEP2, PROS) | 0.91 |
| 93 | (KLKB1, AFAM, FCGBP) | 0.91 |
| 94 | (APOL1, HEP2, PROS) | 0.91 |
| 95 | (CHLE, C4BPB, LUM) | 0.91 |
| 96 | (C4BPB, AFAM, ECM1) | 0.91 |
| 97 | (APOL1, APOA4, C4BPB) | 0.91 |
| 98 | (AMBP, THBG, C4BPB) | 0.91 |
| 99 | (CHLE, C4BPB, FCGBP) | 0.91 |
| 100 | (AMBP, PLF4, HEP2) | 0.91 |
| 101 | (CO8B, C4BPB, AFAM) | 0.91 |
| 102 | (FIBA, VCAM1, FCGBP) | 0.91 |
| 103 | (C1QA, KLKB1, C4BPB) | 0.91 |
| 104 | (APOA4, PROS, CO8B) | 0.91 |
| 105 | (APOL1, THBG, C4BPB) | 0.91 |
| 106 | (THBG, HEP2, C4BPB) | 0.91 |
| 107 | (KLKB1, APOA4, C4BPB) | 0.91 |
| 108 | (THBG, MYL1, C4BPB) | 0.91 |
| 109 | (C4BPB, LUM, FCGBP) | 0.91 |
| 110 | (THBG, HEP2, FCGBP) | 0.91 |
| 111 | (B3AT, CHLE, C4BPB) | 0.91 |
| 112 | (VCAM1, C4BPB, AFAM) | 0.91 |
| 113 | (APOL1, KLKB1, C4BPB) | 0.91 |
| 114 | (C4BPB, LUM, PHLD) | 0.91 |
| 115 | (CHLE, C4BPB, ECM1) | 0.91 |
| 116 | (THBG, C4BPB, HABP2) | 0.91 |
| 117 | (C4BPB, PHLD, ECM1) | 0.91 |
| 118 | (PLF4, HEP2, HABP2) | 0.91 |
| 119 | (AMBP, PROS, HABP2) | 0.91 |
| 120 | (MYL1, CHLE, C4BPB) | 0.91 |
| 121 | (PLF4, HEP2, PROS) | 0.91 |
| 122 | (MYL1, VCAM1, FCGBP) | 0.91 |
| 123 | (HEP2, APOA4, C4BPB) | 0.91 |
| 124 | (PLF4, HEP2, PXDC2) | 0.91 |
| 125 | (HEP2, CHLE, C4BPB) | 0.91 |
| 126 | (AFAM, LUM, FCGBP) | 0.91 |
| 127 | (CERU, PLF4, HEP2) | 0.91 |
| 128 | (PLF4, HEP2, AFAM) | 0.91 |
| 129 | (CHLE, CO8B, C4BPB) | 0.90 |
| 130 | (KLKB1, ECM1, FCGBP) | 0.90 |
| 131 | (VCAM1, ECM1, FCGBP) | 0.90 |
| 132 | (PLF4, LUM, FCGBP) | 0.90 |
| 133 | (KLKB1, VCAM1, C4BPB) | 0.90 |
| 134 | (LUM, PHLD, FCGBP) | 0.90 |
| 135 | (AMBP, PROS, CO8B) | 0.90 |

It was found that a number of cancer biomarkers were unexpectedly overrepresented in the 3plexes, and were deemed as key biomarkers for lung cancer. Some of the key markers in lung cancer include HEP2 that was included in 56 out of the top 135 3plexes, C4BPB that was included in 48 out of the top 135 3plexes, and FCGBP that was included in 45 out of the top 135 3plexes. Among this list of lung cancer 3plexes, the 20 most frequently identified proteins this analysis are listed in Table 9.7

TABLE 9.7
Most common proteins in lung cancer 3plexes

| | Lung | 3plex |
|---|---|---|
| # | cancer | count |
| 1 | HEP2 | 56 |
| 2 | C4BPB | 48 |
| 3 | PROS | 45 |
| 4 | FCGBP | 24 |
| 5 | APOA4 | 21 |
| 6 | PLF4 | 18 |
| 7 | KLKB1 | 18 |
| 8 | LUM | 15 |
| 9 | PHLD | 14 |
| 10 | CHLE | 14 |
| 11 | VCAM1 | 13 |
| 12 | APOL1 | 12 |
| 13 | THBG | 11 |
| 14 | ECM1 | 10 |
| 15 | CO8B | 10 |
| 16 | CERU | 10 |
| 17 | B3AT | 10 |
| 18 | AMBP | 9 |
| 19 | HABP2 | 8 |
| 20 | AFAM | 8 |

The top ranked lung cancer 3plexes (those listed in Table 9.6) may then be selected to generate Logistic regression equations as classifiers for future prediction of samples of unknown cancer/non-cancer status, following the methods outlined above for pan-cancer in Example 8.

Colorectal Cancer Biomarkers and 3plexes

Following the methods above, the colorectal cancer (CRC) cohort was also assessed to identify a list of highly significant proteins as well as the 3plexes based on these proteins which demonstrated accuracy above 0.95. Table 9.8 lists the 53 proteins from the CRC data set that demonstrated highly statistically significant differential expression. As in the previous examples, the most accurate 3plexes were identified from the significant proteins. Table 9.9 lists the 272 3plexes generated from these 53 proteins with accuracy greater than 0.90.

TABLE 9.8
Significantly differentially expressed CRC biomarkers

| Protein | Uniprot AN | Protein Description | p-value | q-value | log2FC | AUC |
|---|---|---|---|---|---|---|
| SRCRL | A1L4H1 | Soluble scavenger receptor | 3.76E−05 | 8.91E−04 | 4.755 | 0.816 |
| | | cysteine-rich domain- | | | | |
| | | containing protein SSC5D | | | | |
| CERU | P00450 | Ceruloplasmin | 1.21E−03 | 1.15E−02 | −0.515 | 0.760 |
| KNG1 | P01042 | Kininogen-1 | 9.80E−03 | 4.55E−02 | −0.561 | 0.840 |
| APOA2 | P02652 | Apolipoprotein A-II | 3.61E−03 | 2.25E−02 | −0.754 | 0.816 |
| B3AT | P02730 | Band 3 anion transport | 4.83E−04 | 6.73E−03 | 4.547 | 0.864 |
| | | protein | | | | |
| C1QA | P02745 | Complement C1q | 1.17E−05 | 4.63E−04 | 1.658 | 0.888 |
| | | subcomponent subunit A | | | | |
| C1QB | P02746 | Complement C1q | 8.18E−04 | 9.24E−03 | 2.827 | 0.952 |
| | | subcomponent subunit B | | | | |
| C1QC | P02747 | Complement C1q | 6.20E−05 | 1.30E−03 | 2.612 | 0.832 |
| | | subcomponent subunit C | | | | |
| CO9 | P02748 | Complement component C9 | 5.27E−03 | 2.95E−02 | −0.450 | 0.752 |
| AMBP | P02760 | Protein AMBP | 1.09E−02 | 4.86E−02 | −0.389 | 0.720 |
| TTHY | P02766 | Transthyretin | 2.05E−03 | 1.67E−02 | −0.967 | 0.864 |
| ALBU | P02768 | Albumin | 5.43E−03 | 2.95E−02 | −0.701 | 0.776 |
| PLF4 | P02776 | Platelet factor 4 | 8.61E−03 | 4.16E−02 | 2.846 | 0.720 |
| KLKB1 | P03952 | Plasma kallikrein | 2.95E−05 | 7.76E−04 | −0.674 | 0.864 |
| THBG | P05543 | Thyroxine-binding globulin | 1.47E−03 | 1.33E−02 | −4.420 | 0.888 |
| HEP2 | P05546 | Heparin cofactor 2 | 1.56E−04 | 2.56E−03 | −1.261 | 0.872 |
| CHLE | P06276 | Cholinesterase | 1.73E−08 | 1.45E−06 | −0.895 | 0.944 |
| GELS | P06396 | Gelsolin | 2.51E−03 | 1.92E−02 | −0.674 | 0.808 |
| APOA4 | P06727 | Apolipoprotein A-IV | 4.26E−11 | 1.01E−08 | −1.573 | 0.952 |
| PROS | P07225 | Vitamin K-dependent protein | 1.41E−04 | 2.56E−03 | 1.150 | 0.888 |
| | | S | | | | |
| CO8B | P07358 | Complement component C8 | 3.85E−03 | 2.34E−02 | −0.484 | 0.760 |
| | | beta chain | | | | |
| TSP1 | P07996 | Thrombospondin-1 | 1.51E−03 | 1.33E−02 | 5.296 | 0.728 |
| ITA2B | P08514 | Integrin alpha-IIb | 7.54E−04 | 9.24E−03 | 5.984 | 0.768 |
| APOA | P08519 | Apolipoprotein(a) | 1.62E−04 | 2.56E−03 | 2.177 | 0.752 |
| A2AP | P08697 | Alpha-2-antiplasmin | 1.86E−03 | 1.57E−02 | −0.474 | 0.720 |
| TFPI1 | P10646 | Tissue factor pathway | 2.79E−03 | 2.02E−02 | 6.018 | 0.772 |
| | | inhibitor | | | | |
| A1AG2 | P19652 | Alpha-1-acid glycoprotein 2 | 7.42E−03 | 3.74E−02 | −0.693 | 0.808 |
| PZP | P20742 | Pregnancy zone protein | 3.03E−03 | 2.05E−02 | 6.466 | 0.612 |
| C4BPB | P20851 | C4b-binding protein beta | 6.57E−05 | 1.30E−03 | 1.151 | 0.776 |
| | | chain | | | | |
| FLNA | P21333 | Filamin-A | 3.50E−03 | 2.24E−02 | 3.706 | 0.720 |
| TENA | P24821 | Tenascin | 6.23E−04 | 8.20E−03 | 3.135 | 0.888 |
| STOM | P27105 | Stomatin | 2.82E−03 | 2.02E−02 | 4.104 | 0.732 |
| PROP | P27918 | Properdin | 7.93E−04 | 9.24E−03 | 0.768 | 0.736 |
| K22E | P35908 | Keratin, type II cytoskeletal 2 | 8.70E−04 | 9.37E−03 | −3.842 | 0.792 |
| | | epidermal | | | | |
| PEDF | P36955 | Pigment epithelium-derived | 7.72E−03 | 3.81E−02 | −0.560 | 0.824 |
| | | factor | | | | |
| BTD | P43251 | Biotinidase | 1.11E−03 | 1.10E−02 | −0.920 | 0.800 |
| AFAM | P43652 | Afamin | 1.59E−06 | 9.40E−05 | −0.898 | 0.880 |
| LUM | P51884 | Lumican | 5.41E−06 | 2.57E−04 | −0.908 | 0.880 |
| HBB | P68871 | Hemoglobin subunit beta | 5.81E−03 | 3.06E−02 | −0.678 | 0.720 |
| HBG1 | P69891 | Hemoglobin subunit gamma- | 5.48E−03 | 2.95E−02 | −2.381 | 0.784 |
| | | 1 | | | | |
| HBA | P69905 | Hemoglobin subunit alpha | 9.00E−03 | 4.26E−02 | −0.630 | 0.720 |
| PHLD | P80108 | Phosphatidylinositol-glycan- | 1.84E−08 | 1.45E−06 | −1.611 | 0.952 |
| | | specific phospholipase D | | | | |
| LG3BP | Q08380 | Galectin-3-binding protein | 2.41E−03 | 1.90E−02 | 0.794 | 0.680 |
| MMRN1 | Q13201 | Multimerin-1 | 3.24E−03 | 2.13E−02 | 4.778 | 0.632 |
| HABP2 | Q14520 | Hyaluronan-binding protein 2 | 2.93E−05 | 7.76E−04 | −2.037 | 0.856 |
| PON3 | Q15166 | Serum | 1.06E−03 | 1.09E−02 | −5.555 | 0.796 |
| | | paraoxonase/lactonase 3 | | | | |
| ECM1 | Q16610 | Extracellular matrix protein 1 | 1.59E−05 | 5.38E−04 | −0.953 | 0.888 |
| PXDC2 | Q6UX71 | Plexin domain-containing | 1.04E−02 | 4.73E−02 | −0.291 | 0.664 |
| | | protein 2 | | | | |
| FHR4 | Q92496 | Complement factor H-related | 5.09E−03 | 2.94E−02 | 1.091 | 0.768 |
| | | protein 4 | | | | |
| AT2A3 | Q93084 | Sarcoplasmic/endoplasmic | 4.52E−03 | 2.68E−02 | 4.878 | 0.716 |
| | | reticulum calcium ATPase 3 | | | | |
| BTBD2 | Q9BX70 | BTB/POZ domain-containing | 7.06E−03 | 3.64E−02 | 3.205 | 0.720 |
| | | protein 2 | | | | |
| HEG1 | Q9ULI3 | Protein HEG homolog 1 | 2.98E−03 | 2.05E−02 | −0.552 | 0.800 |
| FCGBP | Q9Y6R7 | IgGFc-binding protein | 4.77E−04 | 6.73E−03 | 1.008 | 0.768 |

Analysis of every one of the possible 3plexes among the colorectal cancer data resulted in 272 3plexes with accuracy >0.95, which are listed in Table 9.9.

TABLE 9.9
Colorectal Cancer 3plexes with Accuracy >0.95

| | | 5-Fold |
|---|---|---|
| | | average |
| # | 3PLEX | Accuracy |
| 1 | (KNG1, APOA4, PROS) | 1 |
| 2 | (CHLE, PROS, ECM1) | 1 |
| 3 | (HEP2, APOA4, PROS) | 1 |
| 4 | (APOA4, PROS, PON3) | 1 |
| 5 | (CO9, PROS, ECM1) | 1 |
| 6 | (PROS, PEDF, ECM1) | 1 |
| 7 | (KLKB1, APOA4, PROS) | 1 |
| 8 | (CHLE, APOA4, PROS) | 1 |
| 9 | (APOA4, PROS, K22E) | 1 |
| 10 | (TTHY, APOA4, PROS) | 1 |
| 11 | (PROS, ECM1, PXDC2) | 1 |
| 12 | (APOA4, PROS, PEDF) | 1 |
| 13 | (C1QA, GELS, APOA4) | 0.98 |
| 14 | (CO9, APOA4, PROS) | 0.98 |
| 15 | (C1QA, AMBP, HEP2) | 0.98 |
| 16 | (C1QA, C1QB, ECM1) | 0.98 |
| 17 | (C1QB, CO9, ECM1) | 0.98 |
| 18 | (C1QB, C4BPB, ECM1) | 0.98 |
| 19 | (C1QB, APOA4, LUM) | 0.98 |
| 20 | (C1QB, PZP, ECM1) | 0.98 |
| 21 | (C1QB, APOA4, PHLD) | 0.98 |
| 22 | (C1QA, APOA4, PROS) | 0.98 |
| 23 | (HEP2, CHLE, C4BPB) | 0.98 |
| 24 | (KNG1, C1QB, APOA4) | 0.98 |
| 25 | (KNG1, C1QB, ECM1) | 0.98 |
| 26 | (C1QB, CO8B, ECM1) | 0.98 |
| 27 | (C1QB, FLNA, ECM1) | 0.98 |
| 28 | (SRCRL, C1QB, ECM1) | 0.98 |
| 29 | (CERU, PROS, ECM1) | 0.98 |
| 30 | (C1QB, APOA4, TENA) | 0.98 |
| 31 | (APOA4, PROS, ITA2B) | 0.98 |
| 32 | (C1QB, APOA4, STOM) | 0.98 |
| 33 | (C1QB, APOA4, PROP) | 0.98 |
| 34 | (C1QB, KLKB1, APOA4) | 0.98 |
| 35 | (APOA4, PROS, CO8B) | 0.98 |
| 36 | (C1QC, APOA4, PROS) | 0.98 |
| 37 | (THBG, APOA4, PROS) | 0.98 |
| 38 | (C1QB, APOA4, PEDF) | 0.98 |
| 39 | (C1QB, APOA4, BTD) | 0.98 |
| 40 | (C1QB, PLF4, ECM1) | 0.98 |
| 41 | (C1QB, APOA4, AFAM) | 0.98 |
| 42 | (C1QB, APOA4, LG3BP) | 0.98 |
| 43 | (PROS, CO8B, PEDF) | 0.98 |
| 44 | (PROS, CO8B, ECM1) | 0.98 |
| 45 | (C1QB, APOA4, MMRN1) | 0.98 |
| 46 | (C1QB, APOA4, HABP2) | 0.98 |
| 47 | (C1QA, PROS, ECM1) | 0.98 |
| 48 | (C1QB, HABP2, ECM1) | 0.98 |
| 49 | (C1QA, APOA4, STOM) | 0.98 |
| 50 | (PROS, PZP, ECM1) | 0.98 |
| 51 | (C1QB, PON3, ECM1) | 0.98 |
| 52 | (C1QB, ECM1, PXDC2) | 0.98 |
| 53 | (C1QB, ECM1, FHR4) | 0.98 |
| 54 | (C1QB, ECM1, AT2A3) | 0.98 |
| 55 | (C1QB, PROS, ECM1) | 0.98 |
| 56 | (C1QB, ECM1, BTBD2) | 0.98 |
| 57 | (C1QB, TTHY, ECM1) | 0.98 |
| 58 | (C1QB, ECM1, HEG1) | 0.98 |
| 59 | (C1QB, ECM1, FCGBP) | 0.98 |
| 60 | (C1QA, APOA4, PEDF) | 0.98 |
| 61 | (C1QB, A2AP, ECM1) | 0.98 |
| 62 | (C1QB, TFPI1, ECM1) | 0.98 |
| 63 | (B3AT, C1QB, ECM1) | 0.98 |
| 64 | (C1QB, MMRN1, ECM1) | 0.98 |
| 65 | (C1QA, C1QB, APOA4) | 0.98 |
| 66 | (C1QB, APOA4, C4BPB) | 0.98 |
| 67 | (GELS, PROS, ECM1) | 0.98 |
| 68 | (C1QB, LG3BP, ECM1) | 0.98 |
| 69 | (C1QB, APOA4, PON3) | 0.98 |
| 70 | (C1QB, APOA4, ECM1) | 0.98 |
| 71 | (C1QB, APOA4, PXDC2) | 0.98 |
| 72 | (C1QB, APOA4, AT2A3) | 0.98 |
| 73 | (C1QB, ALBU, ECM1) | 0.98 |
| 74 | (C1QB, APOA4, BTBD2) | 0.98 |
| 75 | (C1QB, APOA4, HEG1) | 0.98 |
| 76 | (C1QB, APOA4, FCGBP) | 0.98 |
| 77 | (C1QB, PLF4, APOA4) | 0.98 |
| 78 | (C1QB, PLF4, HEP2) | 0.98 |
| 79 | (PROS, K22E, ECM1) | 0.98 |
| 80 | (APOA4, PROS, TFPI1) | 0.98 |
| 81 | (C1QB, APOA4, FLNA) | 0.98 |
| 82 | (C1QB, APOA4, PZP) | 0.98 |
| 83 | (C1QB, TSP1, ECM1) | 0.98 |
| 84 | (PROS, PHLD, ECM1) | 0.98 |
| 85 | (APOA4, PROS, HABP2) | 0.98 |
| 86 | (B3AT, APOA4, PROS) | 0.98 |
| 87 | (APOA2, C1QB, APOA4) | 0.98 |
| 88 | (APOA4, PROS, PZP) | 0.98 |
| 89 | (APOA4, PROS, ECM1) | 0.98 |
| 90 | (APOA4, PROS, PXDC2) | 0.98 |
| 91 | (C1QB, HEP2, C4BPB) | 0.98 |
| 92 | (C1QB, HEP2, TENA) | 0.98 |
| 93 | (C1QB, ITA2B, ECM1) | 0.98 |
| 94 | (APOA4, PROS, BTBD2) | 0.98 |
| 95 | (ALBU, APOA4, PROS) | 0.98 |
| 96 | (APOA2, C1QB, ECM1) | 0.98 |
| 97 | (PROS, HABP2, ECM1) | 0.98 |
| 98 | (PROS, PON3, ECM1) | 0.98 |
| 99 | (APOA4, PROS, PHLD) | 0.98 |
| 100 | (C1QB, CO9, APOA4) | 0.98 |
| 101 | (C1QB, GELS, APOA4) | 0.98 |
| 102 | (C1QB, AFAM, ECM1) | 0.98 |
| 103 | (C1QB, PROP, ECM1) | 0.98 |
| 104 | (HEP2, PROS, ECM1) | 0.98 |
| 105 | (C1QB, AMBP, APOA4) | 0.98 |
| 106 | (C1QB, BTD, ECM1) | 0.98 |
| 107 | (C1QB, HEP2, ECM1) | 0.98 |
| 108 | (C1QB, CHLE, ECM1) | 0.98 |
| 109 | (C1QB, HEP2, FHR4) | 0.98 |
| 110 | (C1QB, PEDF, ECM1) | 0.98 |
| 111 | (HEP2, PHLD, FCGBP) | 0.98 |
| 112 | (C1QB, AMBP, HEP2) | 0.98 |
| 113 | (C1QB, CHLE, APOA4) | 0.98 |
| 114 | (C1QB, HEP2, TSP1) | 0.98 |
| 115 | (PLF4, APOA4, PROS) | 0.98 |
| 116 | (KNG1, PROS, ECM1) | 0.98 |
| 117 | (C1QB, APOA4, A2AP) | 0.98 |
| 118 | (C1QB, APOA4, CO8B) | 0.98 |
| 119 | (C1QB, APOA4, PROS) | 0.98 |
| 120 | (C1QB, KLKB1, ECM1) | 0.98 |
| 121 | (PROS, BTD, ECM1) | 0.98 |
| 122 | (CERU, C1QB, APOA4) | 0.98 |
| 123 | (APOA4, PROS, PROP) | 0.98 |
| 124 | (C1QB, TENA, ECM1) | 0.98 |
| 125 | (APOA4, K22E, FCGBP) | 0.98 |
| 126 | (C1QB, THBG, APOA4) | 0.98 |
| 127 | (C1QB, APOA, ECM1) | 0.98 |
| 128 | (APOA4, PROS, STOM) | 0.98 |
| 129 | (PROS, AFAM, ECM1) | 0.98 |
| 130 | (APOA4, PROS, FLNA) | 0.98 |
| 131 | (C1QB, TTHY, APOA4) | 0.98 |
| 132 | (KLKB1, PROS, ECM1) | 0.98 |
| 133 | (C1QB, LUM, ECM1) | 0.98 |
| 134 | (SRCRL, C1QB, APOA4) | 0.98 |
| 135 | (C1QB, HEP2, APOA4) | 0.98 |
| 136 | (C1QB, AMBP, ECM1) | 0.98 |
| 137 | (CERU, C1QB, ECM1) | 0.98 |
| 138 | (C1QB, THBG, ECM1) | 0.98 |
| 139 | (C1QB, APOA4, TFPI1) | 0.98 |
| 140 | (B3AT, C1QB, APOA4) | 0.98 |
| 141 | (C1QB, GELS, ECM1) | 0.98 |
| 142 | (C1QB, APOA4, TSP1) | 0.98 |
| 143 | (C1QA, APOA4, BTD) | 0.96 |
| 144 | (C1QA, APOA4, PROP) | 0.96 |
| 145 | (PROS, PROP, ECM1) | 0.96 |
| 146 | (KLKB1, PROS, PEDF) | 0.96 |
| 147 | (CERU, C4BPB, ECM1) | 0.96 |
| 148 | (PROS, ECM1, FCGBP) | 0.96 |
| 149 | (HEP2, C4BPB, PHLD) | 0.96 |
| 150 | (C1QA, APOA4, CO8B) | 0.96 |
| 151 | (PROS, ECM1, BTBD2) | 0.96 |
| 152 | (PROS, ECM1, FHR4) | 0.96 |
| 153 | (C1QA, APOA4, PZP) | 0.96 |
| 154 | (SRCRL, C1QB, HEP2) | 0.96 |
| 155 | (PROS, MMRN1, ECM1) | 0.96 |
| 156 | (AMBP, PROS, ECM1) | 0.96 |
| 157 | (PROS, LUM, ECM1) | 0.96 |
| 158 | (C1QB, C1QC, APOA4) | 0.96 |
| 159 | (C4BPB, K22E, ECM1) | 0.96 |
| 160 | (PROS, PEDF, AFAM) | 0.96 |
| 161 | (PROS, PHLD, FCGBP) | 0.96 |
| 162 | (TTHY, CHLE, PROS) | 0.96 |
| 163 | (C1QB, C1QC, ECM1) | 0.96 |
| 164 | (C1QA, APOA4, ITA2B) | 0.96 |
| 165 | (C1QC, PROS, ECM1) | 0.96 |
| 166 | (AMBP, PROS, A1AG2) | 0.96 |
| 167 | (C1QA, ECM1, HEG1) | 0.96 |
| 168 | (SRCRL, PROS, PEDF) | 0.96 |
| 169 | (APOA4, PROS, AFAM) | 0.96 |
| 170 | (APOA4, PROS, LUM) | 0.96 |
| 171 | (APOA4, PROS, HBB) | 0.96 |
| 172 | (APOA4, PROS, HBG1) | 0.96 |
| 173 | (APOA4, PROS, HBA) | 0.96 |
| 174 | (APOA4, PROS, LG3BP) | 0.96 |
| 175 | (APOA4, PROS, MMRN1) | 0.96 |
| 176 | (C1QB, STOM, ECM1) | 0.96 |
| 177 | (SRCRL, APOA4, PROS) | 0.96 |
| 178 | (APOA4, PROS, FHR4) | 0.96 |
| 179 | (APOA4, PROS, AT2A3) | 0.96 |
| 180 | (PROP, PHLD, FCGBP) | 0.96 |
| 181 | (APOA4, PROS, HEG1) | 0.96 |
| 182 | (APOA4, PROS, FCGBP) | 0.96 |
| 183 | (APOA4, C4BPB, HBB) | 0.96 |
| 184 | (APOA4, C4BPB, K22E) | 0.96 |
| 185 | (HEP2, PROS, APOA) | 0.96 |
| 186 | (HEP2, PROS, PHLD) | 0.96 |
| 187 | (HEP2, PROS, FHR4) | 0.96 |
| 188 | (C1QB, K22E, ECM1) | 0.96 |
| 189 | (PROP, K22E, ECM1) | 0.96 |
| 190 | (APOA4, ITA2B, PHLD) | 0.96 |
| 191 | (TTHY, PROS, PEDF) | 0.96 |
| 192 | (APOA4, PROS, BTD) | 0.96 |
| 193 | (PLF4, PROP, ECM1) | 0.96 |
| 194 | (C1QA, APOA4, ECM1) | 0.96 |
| 195 | (PLF4, HEP2, ECM1) | 0.96 |
| 196 | (C1QA, APOA4, BTBD2) | 0.96 |
| 197 | (AFAM, PHLD, FCGBP) | 0.96 |
| 198 | (C1QA, APOA4, HEG1) | 0.96 |
| 199 | (PROS, C4BPB, ECM1) | 0.96 |
| 200 | (C1QA, TTHY, APOA4) | 0.96 |
| 201 | (C1QB, TFPI1, PEDF) | 0.96 |
| 202 | (FLNA, K22E, ECM1) | 0.96 |
| 203 | (GELS, APOA4, PROS) | 0.96 |
| 204 | (C1QA, C1QB, HEP2) | 0.96 |
| 205 | (PROS, A2AP, PEDF) | 0.96 |
| 206 | (PROS, TSP1, ECM1) | 0.96 |
| 207 | (C1QB, A1AG2, ECM1) | 0.96 |
| 208 | (AMBP, APOA4, PROS) | 0.96 |
| 209 | (C1QB, PHLD, ECM1) | 0.96 |
| 210 | (AMBP, CHLE, PROS) | 0.96 |
| 211 | (KLKB1, CHLE, PROS) | 0.96 |
| 212 | (C1QB, HBG1, ECM1) | 0.96 |
| 213 | (C1QA, K22E, ECM1) | 0.96 |
| 214 | (APOA4, PROS, TSP1) | 0.96 |
| 215 | (APOA4, PROS, APOA) | 0.96 |
| 216 | (APOA4, PROS, A2AP) | 0.96 |
| 217 | (APOA4, PROS, C4BPB) | 0.96 |
| 218 | (APOA4, PROS, TENA) | 0.96 |
| 219 | (C1QB, C4BPB, AFAM) | 0.96 |
| 220 | (APOA4, ITA2B, PON3) | 0.96 |
| 221 | (C1QB, HEP2, STOM) | 0.96 |
| 222 | (C1QB, TTHY, STOM) | 0.96 |
| 223 | (C1QB, HEP2, HBA) | 0.96 |
| 224 | (C1QB, HEP2, HBG1) | 0.96 |
| 225 | (C1QB, TTHY, BTBD2) | 0.96 |
| 226 | (APOA2, PHLD, FCGBP) | 0.96 |
| 227 | (PLF4, PROS, HABP2) | 0.96 |
| 228 | (CHLE, PROS, PHLD) | 0.96 |
| 229 | (CHLE, PROS, HEG1) | 0.96 |
| 230 | (C1QB, APOA4, HBG1) | 0.96 |
| 231 | (C1QB, HEP2, PEDF) | 0.96 |
| 232 | (C1QB, HEP2, K22E) | 0.96 |
| 233 | (C1QB, HEP2, PROP) | 0.96 |
| 234 | (C1QA, HEP2, APOA4) | 0.96 |
| 235 | (CHLE, APOA4, C4BPB) | 0.96 |
| 236 | (C1QB, HEP2, A1AG2) | 0.96 |
| 237 | (C1QB, APOA4, HBA) | 0.96 |
| 238 | (C1QB, TTHY, HEP2) | 0.96 |
| 239 | (PLF4, PROS, ECM1) | 0.96 |
| 240 | (CERU, APOA4, PROS) | 0.96 |
| 241 | (APOA2, C1QB, HEP2) | 0.96 |
| 242 | (C1QB, HEP2, APOA) | 0.96 |
| 243 | (C1QB, HEP2, ITA2B) | 0.96 |
| 244 | (C1QB, HEP2, CO8B) | 0.96 |
| 245 | (HEP2, TENA, FHR4) | 0.96 |
| 246 | (C1QB, HEP2, PROS) | 0.96 |
| 247 | (CHLE, PROS, FHR4) | 0.96 |
| 248 | (CHLE, PROS, PEDF) | 0.96 |
| 249 | (C1QB, AMBP, HABP2) | 0.96 |
| 250 | (A2AP, C4BPB, ECM1) | 0.96 |
| 251 | (PHLD, MMRN1, ECM1) | 0.96 |
| 252 | (C1QB, AMBP, ITA2B) | 0.96 |
| 253 | (PHLD, ECM1, FCGBP) | 0.96 |
| 254 | (THBG, PROS, ECM1) | 0.96 |
| 255 | (B3AT, C1QA, APOA4) | 0.96 |
| 256 | (B3AT, APOA4, K22E) | 0.96 |
| 257 | (CHLE, PROS, TSP1) | 0.96 |
| 258 | (C1QB, APOA4, K22E) | 0.96 |
| 259 | (C1QB, TTHY, FLNA) | 0.96 |
| 260 | (C1QB, ALBU, APOA4) | 0.96 |
| 261 | (C1QB, APOA4, ITA2B) | 0.96 |
| 262 | (C1QB, APOA4, A1AG2) | 0.96 |
| 263 | (C1QA, CHLE, ECM1) | 0.96 |
| 264 | (C1QB, HEP2, FCGBP) | 0.96 |
| 265 | (C1QB, AMBP, CHLE) | 0.96 |
| 266 | (THBG, CHLE, PROS) | 0.96 |
| 267 | (C1QB, HEP2, MMRN1) | 0.96 |
| 268 | (C1QB, AMBP, PEDF) | 0.96 |
| 269 | (C1QB, HEP2, LG3BP) | 0.96 |
| 270 | (C1QB, HEP2, PHLD) | 0.96 |
| 271 | (C1QB, APOA4, FHR4) | 0.96 |
| 272 | (C1QB, APOA4, APOA) | 0.96 |

It was found that a number of the cancer biomarkers were unexpectedly overrepresented in the 3plexes predictive for CRC, and were deemed as key biomarkers for CRC. Some of the key biomarkers in colorectal cancer 3plexes include C1QB that was included in 132 out of the top 272 3plexes, APOA4 that was included in 119 out of the top 135 3plexes, PROS that was included in 102 out of the top 135 3plexes, and ECM1 that was included in 93 out of the top 135 3plexes. Among this list of CRC 3plexes, the 20 most frequently identified proteins this analysis are listed in Table 9.10.

TABLE 9.10
Most common proteins in colorectal cancer 3plexes

| | | 3plex |
|---|---|---|
| # | CRC | count |
| 1 | C1QB | 132 |
| 2 | APOA4 | 119 |
| 3 | PROS | 102 |
| 4 | ECM1 | 93 |
| 5 | HEP2 | 39 |
| 6 | C1QA | 23 |
| 7 | CHLE | 17 |
| 8 | PHLD | 16 |
| 9 | PEDF | 15 |
| 10 | C4BPB | 14 |
| 11 | K22E | 12 |
| 12 | FCGBP | 12 |
| 13 | AMBP | 12 |
| 14 | TTHY | 10 |
| 15 | PROP | 9 |
| 16 | PLF4 | 8 |
| 17 | ITA2B | 8 |
| 18 | FHR4 | 8 |
| 19 | CO8B | 7 |
| 20 | AFAM | 7 |

The top ranked CRC 3plexes (those listed in Table 9.9) may then be selected to generate Logistic regression equations as classifiers for future prediction of samples of unknown cancer/non-cancer status, following the methods outlined above for pan-cancer in Example 8.

Breast Cancer Biomarkers and 3plexes

Following the methods above, the breast cancer cohort was assessed to identify highly significant proteins as well as 3plexes that demonstrated high accuracy. Table 9.11 lists the 36 proteins from the breast cancer data set that demonstrated highly statistically significant differential expression and designated as breast cancer biomarkers. As in the previous examples, the most accurate 3plexes were selected from the significant proteins. Table 9.12 lists the 63 3plexes generated from the 36 breast cancer biomarkers listed in Table 9.11, with accuracy greater than 0.90.

TABLE 9.11
Significantly differentially expressed breast cancer biomarkers

| Protein | Uniprot AN | Protein Name | p-value | q-value | log2FC | AUC |
|---|---|---|---|---|---|---|
| AQR | O60306 | RNA helicase aquarius | 3.93E−03 | 2.74E−02 | 5.225 | 0.776 |
| CERU | P00450 | Ceruloplasmin | 1.62E−04 | 4.13E−03 | −0.618 | 0.776 |
| FA9 | P00740 | Coagulation factor IX | 1.35E−03 | 1.72E−02 | −0.751 | 0.760 |
| FA10 | P00742 | Coagulation factor X | 1.13E−04 | 3.25E−03 | −0.930 | 0.856 |
| AACT | P01011 | Alpha-1-antichymotrypsin | 2.00E−05 | 1.15E−03 | −0.623 | 0.856 |
| FIBA | P02671 | Fibrinogen alpha chain | 5.67E−06 | 4.34E−04 | 0.759 | 0.848 |
| FIBG | P02679 | Fibrinogen gamma chain | 3.86E−03 | 2.74E−02 | 0.473 | 0.768 |
| B3AT | P02730 | Band 3 anion transport protein | 1.01E−03 | 1.51E−02 | 4.541 | 0.848 |
| CRP | P02741 | C-reactive protein | 6.23E−04 | 1.19E−02 | −5.573 | 0.784 |
| C1QA | P02745 | Complement C1q | 3.69E−05 | 1.70E−03 | 1.571 | 0.904 |
| | | subcomponent subunit A | | | | |
| CO9 | P02748 | Complement component C9 | 4.44E−06 | 4.34E−04 | −0.813 | 0.928 |
| KLKB1 | P03952 | Plasma kallikrein | 2.99E−03 | 2.55E−02 | −0.468 | 0.752 |
| A1BG | P04217 | Alpha-1B-glycoprotein | 1.12E−03 | 1.51E−02 | −0.581 | 0.760 |
| HEP2 | P05546 | Heparin cofactor 2 | 9.52E−05 | 3.13E−03 | −1.288 | 0.896 |
| CHLE | P06276 | Cholinesterase | 6.59E−03 | 4.21E−02 | −0.374 | 0.680 |
| APOA4 | P06727 | Apolipoprotein A-IV | 5.22E−05 | 2.00E−03 | −0.887 | 0.848 |
| CO8A | P07357 | Complement component C8 | 3.78E−03 | 2.74E−02 | −0.685 | 0.768 |
| | | alpha chain | | | | |
| CO8B | P07358 | Complement component C8 | 8.94E−04 | 1.47E−02 | −0.631 | 0.792 |
| | | beta chain | | | | |
| ITA2B | P08514 | Integrin alpha-IIb | 2.38E−03 | 2.28E−02 | 5.473 | 0.708 |
| CD14 | P08571 | Monocyte differentiation | 4.06E−03 | 2.75E−02 | −0.763 | 0.696 |
| | | antigen CD14 | | | | |
| A2AP | P08697 | Alpha-2-antiplasmin | 5.22E−04 | 1.09E−02 | −0.499 | 0.760 |
| CO7 | P10643 | Complement component C7 | 3.94E−03 | 2.74E−02 | 0.725 | 0.768 |
| CLUS | P10909 | Clusterin | 2.73E−03 | 2.42E−02 | −0.511 | 0.720 |
| VCAM1 | P19320 | Vascular cell adhesion protein | 4.58E−04 | 1.05E−02 | −0.839 | 0.784 |
| | | 1 | | | | |
| A1AG2 | P19652 | Alpha-1-acid glycoprotein 2 | 1.97E−03 | 2.06E−02 | −0.935 | 0.840 |
| PZP | P20742 | Pregnancy zone protein | 1.81E−03 | 2.06E−02 | 6.595 | 0.568 |
| C4BPB | P20851 | C4b-binding protein beta chain | 8.67E−04 | 1.47E−02 | 0.896 | 0.744 |
| PROP | P27918 | Properdin | 2.12E−03 | 2.12E−02 | 0.715 | 0.768 |
| AFAM | P43652 | Afamin | 3.56E−03 | 2.74E−02 | −0.460 | 0.736 |
| LUM | P51884 | Lumican | 4.52E−03 | 2.97E−02 | −0.473 | 0.696 |
| PHLD | P80108 | Phosphatidylinositol-glycan- | 1.82E−07 | 4.18E−05 | −1.614 | 0.936 |
| | | specific phospholipase D | | | | |
| HABP2 | Q14520 | Hyaluronan-binding protein 2 | 1.08E−03 | 1.51E−02 | −1.680 | 0.856 |
| LTBP1 | Q14766 | Latent-transforming growth | 2.53E−03 | 2.33E−02 | 5.356 | 0.696 |
| | | factor beta-binding protein 1 | | | | |
| ADIPO | Q15848 | Adiponectin | 1.94E−03 | 2.06E−02 | 0.689 | 0.720 |
| APMAP | Q9HDC9 | Adipocyte plasma membrane- | 1.54E−03 | 1.87E−02 | −0.702 | 0.784 |
| | | associated protein | | | | |
| FCGBP | Q9Y6R7 | IgGFc-binding protein | 3.70E−03 | 2.74E−02 | 0.799 | 0.728 |

Analysis of every one of the possible 3plexes among the breast cancer data resulted in 63 3plexes with accuracy >0.9, which are listed in Table 9.12.

TABLE 9.12
Breast Cancer 3plexes with Accuracy >0.90

| | | 5-Fold average |
|---|---|---|
| | 3PLEX | Accuracy |
| 1 | (FIBA, CRP, VCAM1) | 0.96 |
| 2 | (FA9, A1AG2, FCGBP) | 0.96 |
| 3 | (FIBA, HEP2, PHLD) | 0.96 |
| 4 | (B3AT, HEP2, C4BPB) | 0.94 |
| 5 | (FIBG, APOA4, PHLD) | 0.94 |
| 6 | (FIBA, CO8B, PHLD) | 0.94 |
| 7 | (HEP2, VCAM1, FCGBP) | 0.94 |
| 8 | (FIBA, CO9, A1AG2) | 0.94 |
| 9 | (FIBG, HEP2, ADIPO) | 0.94 |
| 10 | (A1AG2, PHLD, FCGBP) | 0.94 |
| 11 | (C1QA, HEP2, PZP) | 0.94 |
| 12 | (FIBG, B3AT, HEP2) | 0.94 |
| 13 | (FIBA, A1AG2, PHLD) | 0.94 |
| 14 | (A1AG2, PHLD, LTBP1) | 0.94 |
| 15 | (FIBA, CRP, PHLD) | 0.94 |
| 16 | (AACT, FIBA, CRP) | 0.94 |
| 17 | (CRP, APOA4, ADIPO) | 0.94 |
| 18 | (CLUS, A1AG2, FCGBP) | 0.94 |
| 19 | (A1AG2, C4BPB, PHLD) | 0.94 |
| 20 | (FA10, FIBG, PHLD) | 0.94 |
| 21 | (FIBG, CRP, VCAM1) | 0.94 |
| 22 | (FIBG, A1BG, PHLD) | 0.92 |
| 23 | (HEP2, APOA4, CO7) | 0.92 |
| 24 | (HEP2, LUM, FCGBP) | 0.92 |
| 25 | (FIBA, CD14, A1AG2) | 0.92 |
| 26 | (AACT, FIBG, PHLD) | 0.92 |
| 27 | (AACT, FIBA, C4BPB) | 0.92 |
| 28 | (FIBG, VCAM1, PHLD) | 0.92 |
| 29 | (CERU, FIBA, PZP) | 0.92 |
| 30 | (FIBG, CO8B, PHLD) | 0.92 |
| 31 | (AACT, FIBA, A1AG2) | 0.92 |
| 32 | (CO8B, PHLD, ADIPO) | 0.92 |
| 33 | (FIBA, CO8B, CD14) | 0.92 |
| 34 | (AACT, C1QA, A1AG2) | 0.92 |
| 35 | (FIBG, HEP2, A2AP) | 0.92 |
| 36 | (FA9, FIBG, PHLD) | 0.92 |
| 37 | (C1QA, APOA4, ADIPO) | 0.92 |
| 38 | (FIBG, CRP, APOA4) | 0.92 |
| 39 | (C1QA, HEP2, C4BPB) | 0.92 |
| 40 | (C1QA, CO9, A1AG2) | 0.92 |
| 41 | (FA9, FIBG, PROP) | 0.92 |
| 42 | (HEP2, PHLD, FCGBP) | 0.92 |
| 43 | (B3AT, HEP2, PZP) | 0.92 |
| 44 | (CO7, PHLD, LTBP1) | 0.92 |
| 45 | (C1QA, A1AG2, PHLD) | 0.92 |
| 46 | (FIBG, HEP2, ITA2B) | 0.92 |
| 47 | (FIBA, APOA4, ADIPO) | 0.92 |
| 48 | (AQR, APOA4, ADIPO) | 0.92 |
| 49 | (HEP2, CO7, PZP) | 0.92 |
| 50 | (FIBA, CRP, APOA4) | 0.92 |
| 51 | (AACT, FIBG, ADIPO) | 0.92 |
| 52 | (APOA4, PZP, ADIPO) | 0.92 |
| 53 | (FIBG, APOA4, ADIPO) | 0.92 |
| 54 | (FIBG, HEP2, PHLD) | 0.92 |
| 55 | (FA9, FIBA, LTBP1) | 0.92 |
| 56 | (APOA4, LTBP1, ADIPO) | 0.92 |
| 57 | (FIBA, CD14, CLUS) | 0.92 |
| 58 | (FA9, FIBA, PZP) | 0.92 |
| 59 | (B3AT, PHLD, ADIPO) | 0.92 |
| 60 | (FIBA, VCAM1, C4BPB) | 0.92 |
| 61 | (FIBG, HEP2, VCAM1) | 0.92 |
| 62 | (FIBA, PHLD, ADIPO) | 0.92 |
| 63 | (FIBA, HEP2, ADIPO) | 0.92 |

It was found that a number of cancer biomarkers were unexpectedly overrepresented in the 3plexes, and were deemed as key biomarkers. Some of the key markers in breast cancer include PHLD that was included in 21 out of the top 63 3plexes, FIBA that was included in 20 out of the top 63 3plexes, and FIBG that was included in 18 out of the top 63 3plexes. Among this list of breast cancer 3plexes, the 20 most frequently identified proteins this analysis are listed in Table 9.13.

TABLE 9.13
Most common proteins in breast cancer 3plexes

| | Breast | 3plex |
|---|---|---|
| # | Cancer | count |
| 1 | PHLD | 21 |
| 2 | FIBA | 20 |
| 3 | FIBG | 18 |
| 4 | HEP2 | 17 |
| 5 | ADIPO | 13 |
| 6 | A1AG2 | 12 |
| 7 | APOA4 | 11 |
| 8 | CRP | 7 |
| 9 | VCAM1 | 6 |
| 10 | PZP | 6 |
| 11 | FCGBP | 6 |
| 12 | C1QA | 6 |
| 13 | AACT | 6 |
| 14 | FA9 | 5 |
| 15 | C4BPB | 5 |
| 16 | LTBP1 | 4 |
| 17 | CO8B | 4 |
| 18 | B3AT | 4 |
| 19 | CO7 | 3 |
| 20 | CD14 | 3 |

The top ranked breast 3plexes (those listed in Table 9.12) may then be selected to generate Logistic regression equations as classifiers for future prediction of samples of unknown cancer/non-cancer status, following the methods outlined above for pan-cancer in Example 8.

Consensus Pan Cancer Biomarkers and 3plexes

Following the generation of accurately predictive 3plexes and key cancer biomarkers from each individual cancer indication, identifying a subset of the pan cancer biomarkers (as listed on Table 8.2) that were significantly differentially expressed across each of the 4 indications individually (ovarian, breast, colorectal and lung cancer) as well as the pan cancer setting, was studied. This study yielded 13 proteins, which may be referred to herein as consensus pan cancer biomarkers meeting these criteria, listed in Table 9.14. That there would be consensus pan cancer biomarkers across multiple cancer types was not expected. The identity of the particular biomarkers that qualified as consensus was also unexpected because they were not the best performing markers in pan cancer or any one cancer type. These markers are expected to be useful for diagnosis and prognostication of not just the particular cancer types from which the quantification data was obtained, but in cancer generally.

TABLE 9.14
“Consensus” pan cancer biomarkers that are significantly
differentially expressed across all tested comparisons

| Protein | Uniprot AN | Protein Name | p-value | q-value | log2FC | AUC |
|---|---|---|---|---|---|---|
| B3AT | P02730 | Band 3 anion transport protein | 4.19E−04 | 3.79E−03 | 4.601 | 0.840 |
| C1QA | P02745 | Complement C1q | 5.38E−05 | 7.91E−04 | 1.474 | 0.859 |
| | | subcomponent subunit A | | | | |
| C4BPB | P20851 | C4b-binding protein beta chain | 5.30E−05 | 7.91E−04 | 1.101 | 0.823 |
| FCGBP | Q9Y6R7 | IgGFc-binding protein | 6.33E−04 | 4.91E−03 | 0.898 | 0.762 |
| CO8B | P07358 | Complement component C8 | 3.09E−05 | 5.59E−04 | −0.574 | 0.739 |
| | | beta chain | | | | |
| CERU | P00450 | Ceruloplasmin | 7.24E−07 | 2.43E−05 | −0.597 | 0.751 |
| KLKB1 | P03952 | Plasma kallikrein | 1.45E−08 | 1.13E−06 | −0.711 | 0.846 |
| CHLE | P06276 | Cholinesterase | 4.75E−08 | 2.34E−06 | −0.733 | 0.829 |
| AFAM | P43652 | Afamin | 3.05E−06 | 8.68E−05 | −0.768 | 0.779 |
| HEP2 | P05546 | Heparin cofactor 2 | 4.98E−08 | 2.34E−06 | −1.182 | 0.847 |
| APOA4 | P06727 | Apolipoprotein A-IV | 1.52E−12 | 3.58E−10 | −1.422 | 0.892 |
| PHLD | P80108 | Phosphatidylinositol-glycan- | 9.11E−05 | 1.20E−03 | −2.009 | 0.918 |
| | | specific phospholipase D | | | | |
| HABP2 | Q14520 | Hyaluronan-binding protein 2 | 3.04E−05 | 5.59E−04 | −2.018 | 0.842 |

Next, all possible 3plexes in the consensus pan cancer biomarker group were assessed, and 13 3plexes with greater than 90% accuracy to correctly classify tumor vs normal patient plasma were identified, which is shown in Table 9.15.

TABLE 9.15
3plexes identified in consensus pan cancer biomarkers

| | | 5-Fold average |
|---|---|---|
| # | 3PLEX | Accuracy |
| 1 | (C4BPB, CHLE, HEP2) | 0.933 |
| 2 | (C4BPB, CERU, PHLD) | 0.925 |
| 3 | (B3AT, C1QA, HABP2) | 0.925 |
| 4 | (B3AT, HEP2, APOA4) | 0.925 |
| 5 | (C4BPB, HEP2, PHLD) | 0.925 |
| 6 | (B3AT, HEP2, PHLD) | 0.925 |
| 7 | (FCGBP, CERU, PHLD) | 0.916 |
| 8 | (B3AT, C4BPB, HEP2) | 0.916 |
| 9 | (C4BPB, HEP2, APOA4) | 0.916 |
| 10 | (B3AT, CHLE, HEP2) | 0.908 |
| 11 | (FCGBP, AFAM, HEP2) | 0.908 |
| 12 | (C1QA, C4BPB, HEP2) | 0.907 |
| 13 | (C1QA, CHLE, HEP2) | 0.907 |

It was found that a number of cancer biomarkers were unexpectedly overrepresented in the 3plexes for the pan cancer consensus 3plexes, and were deemed as key biomarkers. Some of the key biomarkers in the pan-cancer consensus list include HEP2 that was included in 10 out of the top 13 3plexes, C4BPB that was included in 6 out of the top 13 3plexes, B3AT that was included in 5 out of the top 13 3plexes. Among this list of consensus cancer biomarker 3plexes, the most frequently identified proteins in the 3plexes are listed in Table 9.16.

TABLE 9.16
Most common proteins in pan cancer consensus 3plexes

| | | 3plex |
|---|---|---|
| # | Consensus | count |
| 1 | HEP2 | 10 |
| 2 | C4BPB | 6 |
| 3 | B3AT | 5 |
| 4 | PHLD | 4 |

The top ranked consensus pan cancer 3plexes (those listed in Table 9.15) may then be selected to generate Logistic regression equations as classifiers for future prediction of samples of unknown cancer/non-cancer status, following the methods outlined above for pan-cancer in Example 8.

Finally, accuracy, F1, and AUC were calculated for each group of significantly differentially expressed protein group using SVM Linear, and is presented in Table 9.17.

To test the accuracy of the 6 sets of cancer biomarkers (identified as noted above for pan cancer, consensus, lung cancer, CRC, breast cancer, and ovarian cancer), Linear SVM was used with 5-Fold 100 Repeats Stratified Cross validation. For each indication, performance metrics ‘Accuracy’, ‘F1 score’ and ‘AUC’ average along with 95% confidence interval are shown in Table 9.17. Accuracy measures how many observations, both positive and negative, were correctly classified. Accuracy of 0 means the model always predicts the wrong label, whereas accuracy of 1 means that it always predicts the correct label. An accuracy of 0.9 means that the model is expected to predict the correct label in 90% of observations. F1 score is a measure of the harmonic mean of precision and recall, it is a metric for evaluating how the model performed at predicting a positive class (i.e., cancer) in an imbalanced dataset. F1 score is between 0 and 1, an F1 score closer to 1 indicates high precision and recall for a model. AUC score (as described above) is a single number that summarizes the model's performance across all possible classification thresholds. In Table 9.17, the AUC is presented with a maximum score of 1, with 1 indicating perfect predictability, 0.5 indicating lack of predictability, and 0 indication perfectly anticorrelated prediction. The higher the values of Accuracy, F1 and AUC, the better the model is performing in classifying cancer vs normal.

TABLE 9.17
Performance Metrics using Linear SVM

| Linear SVM 5-Fold 100 | | | |
|---|---|---|---|
| Repeats Cross validation | | | |
| | Accuracy | F1 | AUC |
| Pan Cancer | 0.893:(0.75- | 0.931:(0.833- | 0.935:(0.784- |
| | 1.0) | 1.0) | 1.0) |
| Pan Cancer | 0.956:(0.87- | 0.971:(0.914- | 0.982:(0.916- |
| (consensus) | 1.0) | 1.0) | 1.0) |
| Ovarian Cancer | 0.919:(0.7- | 0.912:(0.727- | 0.971:(0.84- |
| | 1.0) | 1.0) | 1.0) |
| Ovarian Cancer | 0.957:(0.8- | 0.956:(0.8-1.0) | 0.982:(0.88- |
| (consensus) | 1.0) | | 1.0) |
| Breast Cancer | 0.943:(0.7- | 0.934:(0.667- | 0.987:(0.88- |
| | 1.0) | 1.0) | 1.0) |
| Breast Cancer | 0.906:(0.7- | 0.894:(0.667- | 0.94:(0.8- |
| (consensus) | 1.0) | 1.0) | 1.0) |
| Lung Cancer | 0.861:(0.667- | 0.825:(0.571- | 0.936:(0.733- |
| | 1.0) | 1.0) | 1.0) |
| Lung Cancer | 0.912:(0.75- | 0.886:(0.667- | 0.948:(0.75- |
| (consensus) | 1.0) | 1.0) | 1.0) |
| CRC | 0.926: (0.7- | 0.928:(0.75-1.0) | 0.969:(0.84- |
| | 1.0) | | 1.0) |
| CRC (consensus) | 0.995:(0.9- | 0.994:(0.889- | 1.0:(1.0-1.0) |
| | 1.0) | 1.0) | |

1. A method for analyzing a biological fluid sample of a subject, the method comprising:
(a) providing a microparticle preparation prepared from a biological fluid sample from a subject, wherein the biological fluid sample comprises microparticles;
(b) assaying the expression level of two or more proteins from the microparticle preparation, to yield a data set comprising respective quantitative measures of each of the two or more proteins;
(c) inputting the data set to a trained classifier that is configured to generate a classification of said sample as positive or negative for a cancer at an accuracy of at least 80%; and
(d) electronically outputting a report that identifies said classification of the sample as positive or negative for the cancer.

2. (canceled)

3. The method of claim 1, wherein the trained classifier was trained with training data obtained from a plurality of training samples, and wherein the training samples are microparticle preparations obtained from biological fluid samples from known cancer patients and known non-cancer subjects.

4. The method of claim 2, wherein the training data set comprises, for each of the plurality of training samples: (a) a training classification of cancer or non-cancer; and (b) a quantitative measure of at least the two or more proteins.

5. The method of claim 4, wherein the trained classifier is an algorithm comprising a plurality of coefficients, each of the plurality of the coefficients being associated with one of the two or more proteins, and wherein the algorithm is configured to generate the classification based on the data set comprising the respective quantitative measures of the two or more proteins and the plurality of coefficients.

6-8. (canceled)

9. The method of claim 1, wherein the providing of the microparticle preparation comprises a use of one or more enrichment processes selected from the group consisting of: centrifugation, ultracentrifugation, density gradients, affinity purification, filtration, electroporation, affinity binding in solution or solid phase, magnetic activated sorting, immunoprecipitation, microfiltration, size-exclusion chromatography, and alternating current (AC) electrokinetic separation.

10. The method of claim 9, wherein the providing of the microparticle preparation comprises use of size-exclusion chromatography, and the microparticles are eluted from a size exclusion chromatography column comprising a solid phase, using water as a mobile phase.

11. (canceled)

12. The method of claim 10, wherein the water is distilled water.

13-48. (canceled)

49. The method of claim 1, wherein the two or more proteins comprises a lipid metabolism protein.

50. (canceled)

51. The method of claim 1, wherein the two or more proteins comprise a hemostasis protein.

52. (canceled)

53. The method of claim 1, wherein the two or more proteins comprise an extracellular matrix protein.

54. (canceled)

55. The method of claim 1, wherein the two or more proteins comprise an innate immunity protein.

56-57. (canceled)

58. The method of claim 1, the method further comprising: (e) determining whether the subject is a candidate for receiving a cancer therapy based on the classification.

59. The method of claim 58, wherein the subject is the candidate, and the method further comprises treating the subject with the cancer therapy.

60. A method of monitoring cancer treatment in a subject, the method comprising:
(a) assessing a biological fluid sample from a subject that previously was receiving a cancer therapy, in accordance with claim 1 to receive a classification of said sample as positive or negative for the cancer; and
(b) selecting the subject to be a candidate to:
i) receive at least one additional administration of the cancer therapy based on the classification; or
ii) receive at least one dose of a different therapeutic agent based on the classification.

61-108. (canceled)

109. A method for determining presence of a cancer-induced host immunomodulated environment in a subject, the method comprising:
(a) providing a microparticle preparation from a biological fluid sample from the subject;
(b) quantifying two or more proteins in the microparticle preparation, wherein the two or more proteins include at least one antigen presenting cell (APC) marker or at least one tumor immune suppressor; and
(c) based on the quantification of the two or more proteins, determining the presence of the cancer-induced immunomodulation in the subject.

110. The method of claim 109, further comprising,
(d). administering an effective amount of an immune response modulator to the subject based on the determination of the presence of cancer-induced immunomodulation in the subject, thereby treating the cancer.

111-112. (canceled)

113. The method of claim 109, wherein the cancer-induced immunomodulation is a cancer-induced immunosuppression.

114-117. (canceled)

118. The method of claim 109, wherein the providing of the microparticle-preparation comprises use of size-exclusion chromatography, and the microparticles are eluted from a size exclusion chromatography column comprising a solid phase, using water as a mobile phase.

119. The method of claim 118, wherein the water is distilled water.

120-121. (canceled)

122. A method comprising:
a) providing a plurality of microparticle preparations, each of the plurality of microparticle preparations being prepared from a plasma or serum sample from one of a plurality of subjects, the plurality of subjects comprising cancer patients and non-cancer subjects;
b) using mass spectrometry, determining quantitative measures of a plurality of proteins in each of the plurality of microparticle preparations
c) preparing a training data set indicating, for each sample, values indicating:
(i) classification of cancer class or non-cancer class; and
(ii) quantitative measures, respectively, of the plurality of proteins; and
d) training a classifier on the training data set, wherein training generates one or more classification rules that classify a new sample as belonging to the cancer class or the non-cancer class.

123-124. (canceled)
